(19) United States
(12) Patent Application Publication
(10) Pub. No.: US 2026/0182244 A1
(43) Pub. Date: Jun. 25, 2026

(54) LIGHT EMITTING ELEMENT, NITROGEN CONTAINING COMPOUND, AND DISPLAY DEVICE

(71) Applicant: Samsung Display Co., Ltd., Yongin-si (KR)

(72) Inventors: Juhui YUN, Yongin-si (KR); YUJIN KANG, Yongin-si (KR)

(21) Appl. No.: 19/425,170

(22) Filed: Dec. 18, 2025

(30) Foreign Application Priority Data
Dec. 24, 2024 (KR) 10-2024-0196221

(51) Int. Cl. H10K 85/60; C07D 403/14; C09K 11/02; H10K 50/11; H10K 101/20

(52) U.S. Cl. CPC H10K 85/654; C07D 403/14; C09K 11/02; H10K 85/6572; H10K 50/11; H10K 2101/20

(57) ABSTRACT

A light emitting element, a nitrogen-containing compound for a light emitting element, and a display device including a light emitting element are provided. The light emitting element includes a first electrode, a second electrode facing the first electrode, and an emission layer between the first electrode and the second electrode and including a nitrogen-containing compound represented by Formula 1.

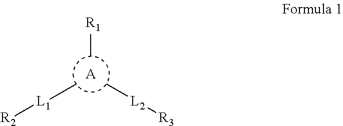

### CROSS-REFERENCE TO RELATED APPLICATION

This application claims priority to and the benefit of Korean Patent Application No. 10-2024-0196221, filed on Dec. 24, 2024 in the Korean Intellectual Property Office, the entire content of which is hereby incorporated by reference.

### BACKGROUND

The present disclosure herein relates to a light emitting element, a nitrogen-containing compound, and a display device, and for example, to a light emitting element, a nitrogen-containing compound used therein, and a display device including the light emitting element.

As image display devices, organic electroluminescence display devices have recently been the focus of active development as image display technologies. Unlike liquid crystal displays and/or similar technologies, organic electroluminescence display devices are self-luminous, in which, holes and electrons injected, respectively, from a first electrode and a second electrode recombine in an emission layer of the organic electroluminescence display device. Subsequently, a light emitting material in the emission layer emits light to achieve or implement display (e.g., of an image). For use in display devices, light emitting elements are desired to exhibit low driving voltage, high luminous efficiency, and long operational lifetime. Accordingly, there is a continuing need or desire for the development of materials for light emitting elements capable of reliably achieving these performance characteristics.

### SUMMARY

One or more aspects of embodiments of the present disclosure are directed toward a light emitting element exhibiting improved luminous efficiency and element service life.

One or more aspects of embodiments of the present disclosure are directed toward a nitrogen-containing compound capable of improving luminous efficiency and element service life.

One or more aspects of embodiments of the present disclosure are directed toward an electronic device including a light emitting element having improved luminous efficiency and service life, and thus exhibiting high display quality.

Additional aspects will be set forth in part in the description which follows and, in part, will be apparent from the description, or may be learned by practice of the presented embodiments of the disclosure.

One or more embodiments of the disclosure provide a light emitting element including a first electrode, a second electrode arranged on the first electrode, and an emission layer arranged between the first electrode and the second electrode and including a first compound represented by Formula 1.

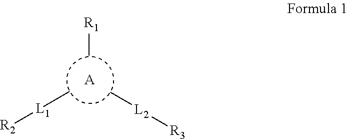

In Formula 1, ring A may be a substituted or unsubstituted heterocycle having 3 ring-forming carbon atoms, three ring-forming heteroatoms selected from among a nitrogen atom, an oxygen atom, and a sulfur atom (e.g., as ring-forming atoms), and at least one nitrogen atom as a ring-forming atom. In Formula 1, L₁ and L₂ may each independently be a substituted or unsubstituted heteroarylene group having 6 to 30 ring-forming carbon atoms, and including at least one nitrogen atom as a ring-forming atom. In Formula 1, R₁ may be a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms, and R₂ and R₃ may each independently be represented by Formula S-1 or Formula S-2.

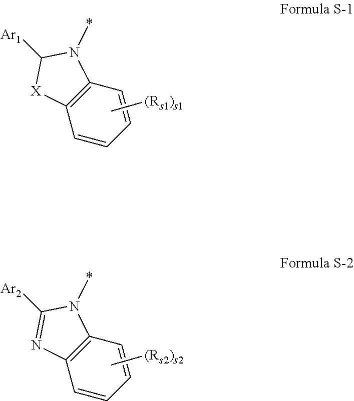

In Formulas S-1 and S-2, X may be NRₛ₃, O, or S, Ar₁ and Ar₂ may each independently be a substituted or unsubstituted aryl group having 6 to 60 ring-forming carbon atoms, or a substituted or unsubstituted heteroaryl group having 2 to 60 ring-forming carbon atoms, Rₛ₁ to Rₛ₃ may each independently be a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms, and s1 and s2 may each independently be an integer of 0 to 4. In Formulas S-1 and S-2, [symbol] is a site linked to Formula 1.

In one or more embodiments, the first compound may be represented by Formula 2-1 or Formula 2-2.

In one or more embodiments, the first compound may be represented by Formula 3-1 or Formula 3-2.

In one or more embodiments, the first compound may be represented by Formula 3-1-1 or Formula 3-1-2.

In one or more embodiments, the first compound may be represented by Formula 3-2-1 or Formula 3-2-2.

In one or more embodiments, the first compound may be represented by Formula 4 or Formula 5.

In one or more embodiments, the first compound may be represented by Formula 4-1 or Formula 4-2.

In one or more embodiments, the emission layer may include a host and a dopant, and the host may contain the first compound.

In one or more embodiments, the emission layer may be to emit thermally activated delayed fluorescence.

In one or more embodiments of the disclosure, provided is a nitrogen-containing compound represented by Formula 1.

In one or more embodiments of the disclosure, an electronic device comprises a display device, wherein the display device includes a base layer, a circuit layer arranged on the base layer, and a display element layer arranged on the circuit layer and including a light emitting element, wherein the light emitting element includes a first electrode, a second electrode arranged on the first electrode, and an emission layer arranged between the first electrode and the second electrode, and the emission layer includes a nitrogen-containing compound represented by Formula 1. For example, the incorporation of specific nitrogen-containing heterocycles into emissive compounds has demonstrated potential to enhance charge transport characteristics and exciton management within the emission layer. The present disclosure addresses these technical challenges by providing a class of nitrogen-containing compounds that exhibit favorable electronic properties and structural stability, thereby contributing to improved luminous efficiency and operational lifetime of light emitting elements. These compounds are particularly suitable for use in thermally activated delayed fluorescence (TADF) systems, where control over molecular energy levels and excited-state dynamics is desired for efficient light emission. The disclosed compounds and devices thus represent a targeted approach to enhance the performance of organic electroluminescent display technologies.

### BRIEF DESCRIPTION OF THE DRAWINGS

The accompanying drawings are included to provide a further understanding of the disclosure, and are incorporated in and constitute a part of this specification. The drawings illustrate example embodiments of the disclosure and, together with the description, serve to explain principles of the disclosure. In the drawings:

FIG. 1 is a block diagram of an electronic device according to one or more embodiments;

FIG. 2 shows schematic views of electronic devices according to one or more embodiments;

FIG. 3 is a plan view of a display device according to one or more embodiments of the disclosure;

FIG. 4 is a cross-sectional view of a display device according to one or more embodiments of the disclosure;

FIG. 5 is a cross-sectional view schematically showing a light emitting element according to one or more embodiments of the disclosure;

FIG. 6 is a cross-sectional view schematically showing a light emitting element according to one or more embodiments of the disclosure;

FIG. 7 is a cross-sectional view schematically showing a light emitting element according to one or more embodiments of the disclosure;

FIG. 8 is a cross-sectional view schematically showing a light emitting element according to one or more embodiments of the disclosure;

FIGS. 9 and 10 are each a cross-sectional view of a display device according to one or more embodiments of the disclosure;

FIG. 11 is a cross-sectional view showing a display device according to one or more embodiments of the disclosure;

FIG. 12 is a cross-sectional view showing a display device according to one or more embodiments of the disclosure; and

FIG. 13 is a view showing a vehicle in which a display device according to one or more embodiments is arranged.

### DETAILED DESCRIPTION

The disclosure may be modified in one or more suitable manners and have many forms, and thus specific embodiments will be exemplified in the drawings and described in more detail in the detailed description of the invention. It should be understood, however, that it is not intended to limit the disclosure to the particular forms disclosed, but rather, is intended to cover all modifications, equivalents, and alternatives falling within the spirit and scope of the disclosure.

When explaining each of drawings, like reference numbers are used for referring to like elements. In the accompanying drawings, the dimensions of each structure are exaggeratingly illustrated for clarity of the present disclosure. It will be understood that, although the terms “first,” “second,” and/or the like, may be used herein to describe one or more suitable components, these components should not be limited by these terms. These terms are only used to distinguish one component from another. For example, a first component could be termed a second component, and, similarly, a second component could be termed a first component, without departing from the scope of example embodiments of the disclosure. As used herein, the singular forms, “a,” “an,” and “the” are intended to include the plural forms as well, unless the context clearly indicates otherwise.

In the present application, it will be understood that the terms “include,” “includes,” “including,” “comprise,” “comprises”, “comprising,” “has,” “having,” “have” and/or the like specify the presence of features, numbers, steps, operations, component, parts, or combinations thereof disclosed in the specification, but do not exclude the possibility of presence or addition of one or more other features, numbers, steps, operations, component, parts, or combinations thereof.

In the present application, if (e.g., when) a layer, a film, a region, or a plate is referred to as being “on” or “in an upper portion of” another layer, film, region, or plate, it may be not only “directly on” the layer, film, region, or plate, but intervening layers, films, regions, or plates may also be present. On the contrary to this, if (e.g., when) a layer, a film, a region, or a plate is referred to as being “below”, “in a lower portion of” another layer, film, region, or plate, it can be not only directly under the layer, film, region, or plate, but intervening layers, films, regions, or plates may also be present. In some embodiments, it will be understood that if (e.g., when) a part is referred to as being “on” another part, it can be arranged above the other part, or arranged under the other part as well.

For example, the terms, such as “lower”, “above”, “upper” and/or the like, are utilized herein for ease of description to describe one element's relation to another element(s) as illustrated in the drawings. The terms are relative concepts and are described based on the directions indicated in the drawings. It will be understood that the terms have a relative concept and are described on the basis of the orientation depicted in the drawings. It will be understood that the spatially relative terms are intended to encompass different orientations of the device in use or operation in addition to the orientation depicted in the drawings. For example, if the device in the drawings is turned over, elements described as “beneath” other elements or features would then be oriented “above” or “over” the other elements or features. Thus, the term “beneath” may encompass both an orientation of above and below. The device may be otherwise oriented (rotated 90 degrees or at other orientations), and the spatially relative descriptors used herein should be interpreted accordingly.

As utilized herein, the terms “use,” “using,” and “used” may be considered synonymous with the terms “utilize,” “utilizing,” and “utilized,” respectively.

As utilized herein, expressions such as “at least one of,” “one of,” “selected from,” and “selected from among,” when preceding a list of elements, modify the entire list of elements and do not modify the individual elements of the list. For example, the expressions “at least one of a to c,” “at least one of a, b or c,” and “at least one of a, b and/or c” may indicate only a, only b, only c, both (e.g., simultaneously) a and b, both (e.g., simultaneously) a and c, both (e.g., simultaneously) b and c, all of a, b, and c, or variations thereof.

The term “and/or” includes all combinations of one or more of the associated listed elements.

As utilized herein, the term “may” will be understood to refer to “one or more embodiments of the present disclosure,” some of which include the described element and some of which exclude that element and/or include an alternate element. Similarly, alternative language such as “or” refers to “one or more embodiments of the present disclosure,” each including a corresponding listed item.

Unless otherwise defined, all terms (including chemical, technical and scientific terms) utilized herein have the same meaning as commonly understood by one of ordinary skill in the art to which this present disclosure belongs. It will be further understood that terms, such as those defined in commonly utilized dictionaries, should be interpreted as having a meaning that is consistent with their meaning in the context of the relevant art and will not be interpreted in an idealized or overly formal sense unless expressly so defined herein.

As utilized herein, the phrase “consisting essentially of” refers to that any additional components will not materially affect the chemical, physical, optical, or electrical properties of the semiconductor film.

As utilized herein, the phrase “on a plane,” or “plan view,” refers to viewing a target portion from the top, and the phrase “on a cross-section” refers to viewing a cross-section formed by vertically cutting a target portion from the side.

In present disclosure, “not include a or any ‘component’” “exclude a or any ‘component’”, “‘component’-free”, and/or the like refers to that the “component” not being added, selected or utilized as a component in the composition, but the “component” of less than a suitable amount may still be included due to other impurities and/or external factor.

### Definitions

In the specification, the term “substituted or unsubstituted” may refer to substituted or unsubstituted with at least one substituent selected from the group consisting of a deuterium atom, a halogen atom, a cyano group, a nitro group, an amino group, an amine group, a silyl group, an oxy group, a thio group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In some embodiments, each of the substituents exemplified herein may be substituted or unsubstituted. For example, a biphenyl group may be interpreted as an aryl group or a phenyl group substituted with a phenyl group.

In the specification, the phrase “bonded to an adjacent group to form a ring” may refer to that a group is bonded to an adjacent group to form a substituted or unsubstituted hydrocarbon ring, or a substituted or unsubstituted heterocycle. The hydrocarbon ring includes an aliphatic hydrocarbon ring and an aromatic hydrocarbon ring. The heterocycle includes an aliphatic heterocycle and an aromatic heterocycle.

The hydrocarbon ring and the heterocycle may be monocyclic or polycyclic. In some embodiments, the rings formed by being bonded to each other may be connected to another ring to form a spiro structure.

In the specification, the term “adjacent group” may refer to a substituent substituted for an atom which is directly linked to an atom substituted with a corresponding substituent, another substituent substituted for an atom which is substituted with a corresponding substituent, or a substituent sterically positioned at the nearest position to a corresponding substituent. For example, two methyl groups in 1,2-dimethylbenzene may be interpreted as “adjacent groups” to each other and two ethyl groups in 1,1-diethylcyclopentane may be interpreted as “adjacent groups” to each other. In some embodiments, two methyl groups in 4,5-dimethylphenanthrene may be interpreted as “adjacent groups” to each other.

In the specification, examples of the halogen atom may include a fluorine atom, a chlorine atom, a bromine atom, or an iodine atom.

In the specification, the alkyl group may be linear or branched. The number of carbons in the alkyl group is 1 to 50, 1 to 30, 1 to 20, 1 to 10, or 1 to 6. Examples of the alkyl group may include a methyl group, an ethyl group, an n-propyl group, an isopropyl group, an n-butyl group, an s-butyl group, a t-butyl group, an i-butyl group, a 2-ethylbutyl group, a 3,3-dimethylbutyl group, an n-pentyl group, an i-pentyl group, a neopentyl group, a t-pentyl group, a 1-methylpentyl group, a 3-methylpentyl group, a 2-ethylpentyl group, a 4-methyl-2-pentyl group, an n-hexyl group, a 1-methylhexyl group, a 2-ethylhexyl group, a 2-butylhexyl group, an n-heptyl group, a 1-methylheptyl group, a 2,2-dimethylheptyl group, a 2-ethylheptyl group, a 2-butylheptyl group, an n-octyl group, a t-octyl group, a 2-ethyloctyl group, a 2-butyloctyl group, a 2-hexyloctyl group, a 3,7-dimethyloctyl group, an n-nonyl group, an n-decyl group, an adamantyl group, a 2-ethyldecyl group, a 2-butyldecyl group, a 2-hexyldecyl group, a 2-octyldecyl group, an n-undecyl group, an n-dodecyl group, a 2-ethyldodecyl group, a 2-butyldodecyl group, a 2-hexyldodecyl group, a 2-octyldodecyl group, an n-tridecyl group, an n-tetradecyl group, an n-pentadecyl group, an n-hexadecyl group, a 2-ethylhexadecyl group, a 2-butylhexadecyl group, a 2-hexylhexadecyl group, a 2-octylhexadecyl group, an n-heptadecyl group, an n-octadecyl group, an n-nonadecyl group, an n-eicosyl group, a 2-ethyleicosyl group, a 2-butyleicosyl group, a 2-hexyleicosyl group, a 2-octyleicosyl group, an n-henicosyl group, an n-docosyl group, an n-tricosyl group, an n-tetracosyl group, an n-pentacosyl group, an n-hexacosyl group, an n-heptacosyl group, an n-octacosyl group, an n-nonacosyl group, an n-triacontyl group, and/or the like, but the embodiment of the disclosure is not limited thereto.

In the specification, a cycloalkyl group may refer to a cyclic alkyl group. The number of carbons in the cycloalkyl group is 3 to 50, 3 to 30, 3 to 20, or 3 to 10. Examples of the cycloalkyl group may include a cyclopropyl group, a cyclobutyl group, a cyclopentyl group, a cyclohexyl group, a 4-methylcyclohexyl group, a 4-t-butylcyclohexyl group, a cycloheptyl group, a cyclooctyl group, a cyclononyl group, a cyclodecyl group, a norbornyl group, a 1-adamantyl group, a 2-adamantyl group, an isobornyl group, a bicycloheptyl group, and/or the like, but the embodiment of the disclosure is not limited thereto.

In the specification, an alkenyl group refers to a hydrocarbon group including at least one carbon double bond in the middle or terminal of an alkyl group having 2 or more carbon atoms. The alkenyl group may be linear or branched. The number of carbon atoms in the alkenyl group is not specifically limited, but is 2 to 30, 2 to 20, or 2 to 10. Examples of the alkenyl group include a vinyl group, a 1-butenyl group, a 1-pentenyl group, a 1,3-butadienyl group, a styrenyl group, a styryl vinyl group, and/or the like, but the embodiment of the disclosure is not limited thereto.

In the specification, an alkynyl group refers to a hydrocarbon group including at least one carbon triple bond in the middle or terminal of an alkyl group having 2 or more carbon atoms. The alkynyl group may be linear or branched. Although the number of carbon atoms is not specifically limited, it is 2 to 30, 2 to 20, or 2 to 10. Specific examples of the alkynyl group may include an ethynyl group, a propynyl group, and/or the like, but are not limited thereto.

In the specification, the hydrocarbon ring group refers to any functional group or substituent derived from an aliphatic hydrocarbon ring. The hydrocarbon ring group may be a saturated hydrocarbon ring group having 5 to 30 or 5 to 20 ring-forming carbon atoms.

In the specification, an aryl group refers to any functional group or substituent derived from an aromatic hydrocarbon ring. The aryl group may be a monocyclic aryl group or a polycyclic aryl group. The number of ring-forming carbon atoms in the aryl group may be 6 to 60, 6 to 30, 6 to 20, or 6 to 15. Examples of the aryl group may include a phenyl group, a naphthyl group, a fluorenyl group, an anthracenyl group, a phenanthryl group, a biphenyl group, a terphenyl group, a quaterphenyl group, a quinquephenyl group, a sexiphenyl group, a triphenylenyl group, a pyrenyl group, a benzofluoranthenyl group, a chrysenyl group, and/or the like, but the embodiment of the disclosure is not limited thereto.

In the specification, the fluorenyl group may be substituted, and two substituents may be bonded to each other to form a spiro structure. Examples of the substituted fluorenyl group are as follows. However, the embodiment of the disclosure is not limited thereto.

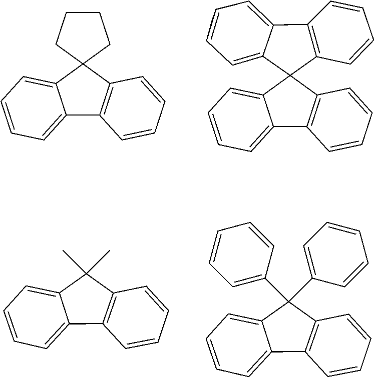

The heterocyclic group herein refers to any functional group or substituent derived from a ring containing at least one of B, O, N, P, Si, Se or S as a heteroatom. Examples of the heterocyclic group includes an aliphatic heterocyclic group and an aromatic heterocyclic group. The aromatic heterocyclic group may be a heteroaryl group. The aliphatic heterocycle and the aromatic heterocycle may each be monocyclic or polycyclic.

In the specification, the heterocyclic group may contain at least one of B, O, N, P, Si or S as a heteroatom. If the heterocyclic group contains two or more heteroatoms, the two or more heteroatoms may be the same as or different from each other. The heterocyclic group may be a monocyclic heterocyclic group or a polycyclic heterocyclic group, and includes a heteroaryl group. The number of ring-forming carbon atoms in the heterocyclic group may be 2 to 60, 2 to 30, 2 to 20, or 2 to 10.

In the specification, the aliphatic heterocyclic group may include at least one of B, O, N, P, Si, Se or S as a heteroatom. The number of ring-forming carbon atoms in the aliphatic heterocyclic group may be 2 to 60, 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, and/or the like, but the embodiment of the disclosure is not limited thereto.

In the specification, the heteroaryl group may contain at least one of B, O, N, P, Si, Se or S as a heteroatom. If the heteroaryl group contains two or more heteroatoms, the two or more heteroatoms may be the same as or different from each other. The heteroaryl group may be a monocyclic heterocyclic group or a polycyclic heterocyclic group. The number of ring-forming carbon atoms in the heteroaryl group may be 2 to 60, 2 to 30, 2 to 20, or 2 to 10. Examples of the heteroaryl group may include a thiophene group, a furan group, a pyrrole group, an imidazole group, a pyridine group, a bipyridine group, a pyrimidine group, a triazine group, a triazole group, an acridyl group, a pyridazine group, a pyrazinyl group, a quinoline group, a quinazoline group, a quinoxaline group, a phenoxazine group, a phthalazine group, a pyrido pyrimidine group, a pyrido pyrazine group, a pyrazino pyrazine group, an isoquinoline group, an indole group, a carbazole group, an N-arylcarbazole group, an N-heteroarylcarbazole group, an N-alkylcarbazole group, a benzoxazole group, a benzimidazole group, a benzothiazole group, a benzocarbazole group, a benzothiophene group, a dibenzothiophene group, a thienothiophene group, a benzofuran group, a phenanthroline group, a thiazole group, an isoxazole group, an oxazole group, an oxadiazole group, a thiadiazole group, a phenothiazine group, a dibenzosilole group, a dibenzofuran group, and/or the like, but the embodiment of the disclosure is not limited thereto.

In the specification, the description of the aryl group may be applied to an arylene group except that the arylene group is a divalent group. The description of the heteroaryl group may be applied to a heteroarylene group except that the heteroarylene group is a divalent group.

In the specification, the silyl group includes an alkylsilyl group and an arylsilyl group. Examples of the silyl group may include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and/or the like, but the embodiment of the disclosure is not limited thereto.

In the specification, the number of carbon atoms in the carbonyl group is not specifically limited, but may be 1 to 40, 1 to 30, or 1 to 20. Examples of the carbonyl group may have the following structures, but the embodiment of the disclosure is not limited thereto.

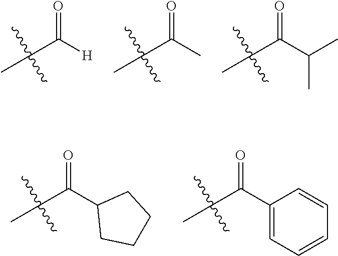

In the specification, the number of carbon atoms in the sulfinyl group and the sulfonyl group is not particularly limited, but may be 1 to 30. Examples of the sulfinyl group may include an alkyl sulfinyl group and an aryl sulfinyl group. Examples of the sulfonyl group may include an alkyl sulfonyl group and an aryl sulfonyl group.

In the specification, the thio group may include an alkylthio group and an arylthio group. The thio group may refer to that a sulfur atom is bonded to the alkyl group or the aryl group as defined. Examples of the thio group may include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, but the embodiment of the disclosure is not limited thereto.

In the specification, an oxy group may refer to that an oxygen atom is bonded to the alkyl group or the aryl group as defined. Examples of the oxy group may include an alkoxy group and an aryl oxy group. The alkoxy group may be a linear chain, a branched chain or a ring chain. The number of carbon atoms in the alkoxy group is not specifically limited, but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, benzyloxy, and/or the like, but the embodiment of the disclosure is not limited thereto.

The boron group herein may refer to that a boron atom is bonded to the alkyl group or the aryl group as defined. Examples of the boron group includes an alkyl boron group and an aryl boron group. Examples of the boron group may include a dimethylboron group, a trimethylboron group, a t-butylmethylboron group, a diphenylboron group, a phenylboron group, and/or the like, but the embodiment of the disclosure is not limited thereto.

In the specification, the number of carbon atoms in an amine group is not specifically limited, but may be 1 to 30. Examples of the amine group may include an alkyl amine group and an aryl amine group. Examples of the amine group may include a methylamine group, a dimethylamine group, a phenylamine group, a diphenylamine group, a naphthylamine group, a 9-methyl-anthracenylamine group, and/or the like, but the embodiment of the disclosure is not limited thereto.

In the specification, the alkyl group among an alkylthio group, an alkylsulfoxy group, an alkylaryl group, an alkylamino group, an alkyl boron group, an alkyl silyl group, and an alkyl amine group is the same as the examples of the alkyl group described herein.

In the specification, the aryl group among an aryloxy group, an arylthio group, an arylsulfoxy group, an arylamino group, an arylboron group, an arylsilyl group, an arylamine group is the same as the examples of the aryl group described herein.

In the specification, a direct linkage may refer to a single bond.

In one or more embodiments, in the specification, and

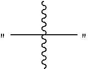

and “ [symbol] ”
refer to a position to be connected.

Hereinafter, embodiments of the disclosure will be described with reference to the accompanying drawings.

### Electronic Device

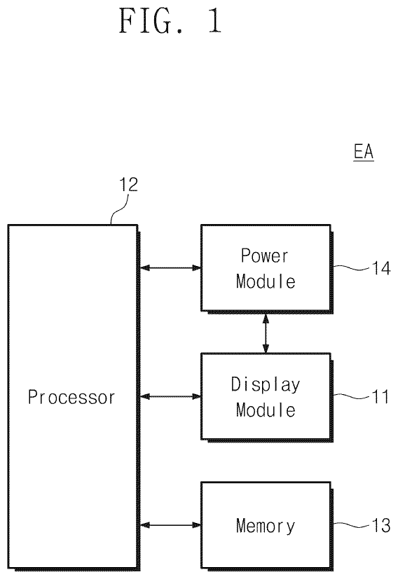

FIG. 1 is a block diagram of an electronic device according to one or more embodiments. Referring to FIG. 1, an electronic device EA according to one or more embodiments may include a display module 11, a processor 12, a memory 13, and a power module 14.

The processor 12 may include at least one of a central processing unit (CPU), an application processor (AP), a graphic processing unit (GPU), a communication processor (CP), an image signal processor (ISP), or a controller.

The memory 13 may store data information desired or required for the operation of the processor 12 or the display module 11. When the processor 12 executes an application stored in the memory 13, image data signals and/or input control signals are transmitted to the display module 11, and the display module 11 may process the received signal and output image information through a display screen. The display module 11 may include a display panel that displays an image.

The power module 14 may include a power supply module such as a power adapter or a battery device, and a power conversion module that converts power supplied by the power supply module to generate power desired or required for the operation of the electronic device EA.

At least one of the components of the electronic device EA described herein may be included in a display panel according to one or more embodiments, which will be described in more detail later, and a display device of one or more embodiments including the same. In some embodiments, some of the individual modules functionally included in one module may be included in the display device, and others may be provided separately from the display device. For example, the display device may include a display module 11, and the processor 12, the memory 13 and the power module 14 may be provided in the form of other devices within the electronic device EA, rather than the display device.

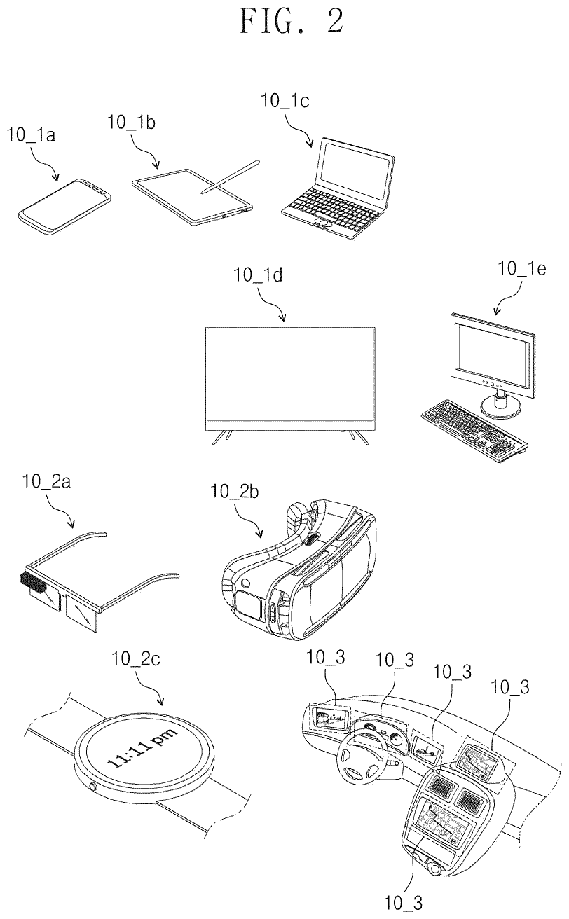

FIG. 2 shows schematic views of electronic devices according to one or more suitable embodiments.

Referring to FIG. 2, one or more suitable electronic devices including the display device according to one or more embodiments may include electronic devices for displaying images, such as a smart phone 10_1a, a tablet PC 10_1b, a laptop 10_1c, a TV 10_1d, and a desk monitor 10_1e, wearable electronic devices such as smart glasses 10_2a, a head mounted display 10_2b, and a smart watch 10_2c, and vehicle electronic devices 10_3 such as a center information display (CID) and a room mirror display arranged on an instrument panel, a center fascia, or a dashboard of a vehicle.

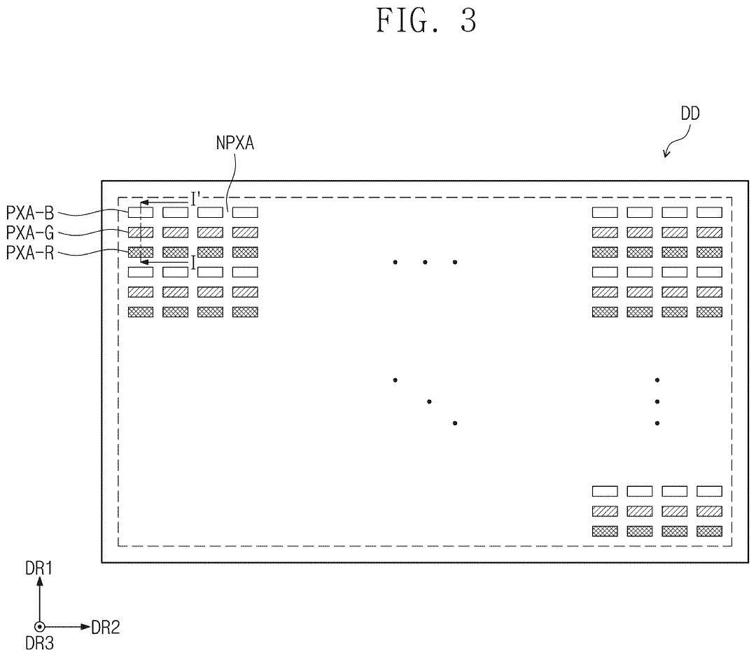

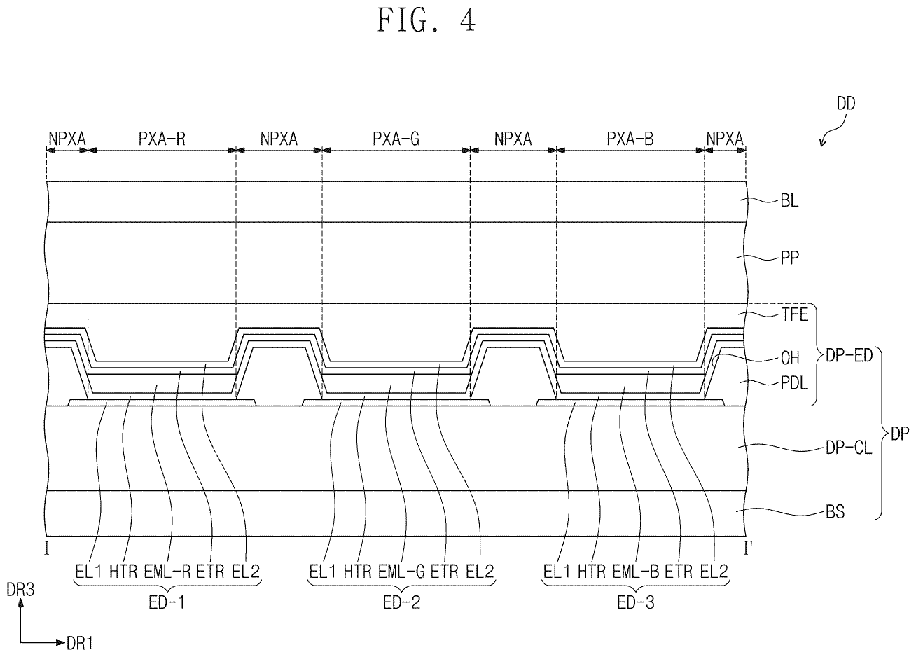

FIG. 3 is a plan view illustrating one or more embodiments of a display device DD. FIG. 4 is a cross-sectional view of the display device DD of the embodiment. FIG. 4 is a cross-sectional view illustrating a part taken along the line I-I′ of FIG. 3. The display device DD of one or more embodiments may be included in the electronic device EA described herein. The display device DD may be a portion of the electronic device EA that provides images.

The display device DD may include a display panel DP and an optical layer PP arranged on the display panel DP. The display panel DP includes light emitting elements ED-1, ED-2, and ED-3. The display device DD may include a plurality of light emitting elements ED-1, ED-2, and ED-3. The optical layer PP may be arranged on the display panel DP to control reflected light in the display panel DP due to external light. The optical layer PP may include, for example, a polarization layer or a color filter layer. In one or more embodiments, unlike the configuration illustrated in the drawing, the optical layer PP may not be provided from the display device DD of one or more embodiments.

A base substrate BL may be arranged on the optical layer PP. The base substrate BL may be a member which provides a base surface on which the optical layer PP arranged. The base substrate BL may be a glass substrate, a metal substrate, a plastic substrate, and/or the like. However, the embodiment of the disclosure is not limited thereto, and the base substrate BL may be an inorganic layer, an organic layer, or a composite material layer. In some embodiments, unlike the configuration illustrated, the base substrate BL may not be provided.

The display device DD according to one or more embodiments may further include a filling layer. The filling layer may be arranged between a display device layer DP-ED and the base substrate BL. The filling layer may be an organic material layer. The filling layer may include at least one of an acrylic-based resin, a silicone-based resin, or an epoxy-based resin.

The display panel DP may include a base layer BS, a circuit layer DP-CL provided on the base layer BS, and the display device layer DP-ED. The display device layer DP-ED may include a pixel defining film PDL, the light emitting elements ED-1, ED-2, and ED-3 arranged between portions of the pixel defining film PDL, and an encapsulation layer TFE arranged on the light emitting elements ED-1, ED-2, and ED-3.

The base layer BS may be a member which provides a base surface on which the display device layer DP-ED is arranged. The base layer BS may be a glass substrate, a metal substrate, a plastic substrate, and/or the like. However, the embodiment is not limited thereto, and the base layer BS may be an inorganic layer, an organic layer, or a composite material layer.

In one or more embodiments, the circuit layer DP-CL is arranged on the base layer BS, and the circuit layer DP-CL may include a plurality of transistors. Each of the transistors may include a control electrode, an input electrode, and an output electrode. For example, the circuit layer DP-CL may include a switching transistor and a driving transistor for driving the light emitting elements ED-1, ED-2, and ED-3 of the display device layer DP-ED.

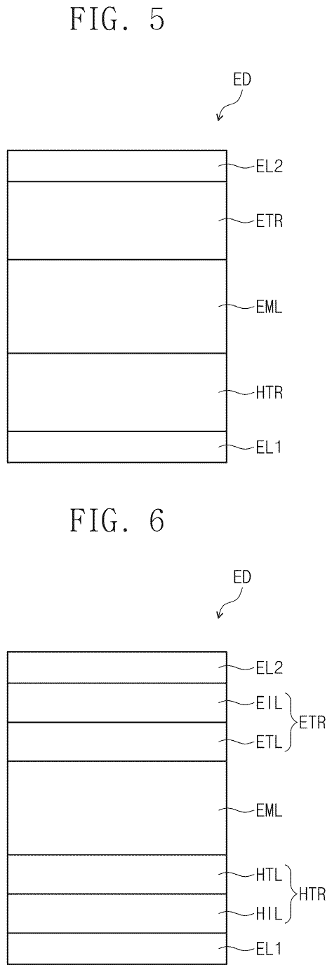

Each of the light emitting elements ED-1, ED-2, and ED-3 may have a structure of each light emitting element ED of embodiments according to FIGS. 5 to 8, which will be described later. Each of the light emitting elements ED-1, ED-2, and ED-3 may include a first electrode EL1, a hole transport region HTR, emission layers EML-R, EML-G, and EML-B, an electron transport region ETR, and a second electrode EL2.

FIG. 4 illustrates an embodiment in which the emission layers EML-R, EML-G, and EML-B of the light emitting elements ED-1, ED-2, and ED-3 are arranged in openings OH defined in the pixel defining film PDL, and the hole transport region HTR, the electron transport region ETR, and the second electrode EL2 are provided as a common layer in the entire light emitting elements ED-1, ED-2, and ED-3. However, the embodiment of the disclosure is not limited thereto, and unlike the configuration illustrated in FIG. 4, the hole transport region HTR and the electron transport region ETR in one or more embodiments may be provided by being patterned inside the openings OH defined in the pixel defining film PDL. For example, the hole transport region HTR, the emission layers EML-R, EML-G, and EML-B, and the electron transport region ETR of the light emitting element ED-1, ED-2, and ED-3 in one or more embodiments may be provided by being patterned in an inkjet printing method.

The encapsulation layer TFE may cover the light emitting elements ED-1, ED-2 and ED-3. The encapsulation layer TFE may seal the light emitting element ED-1, ED-2, and ED-3 in the display device layer DP-ED. The encapsulation layer TFE may be a thin film encapsulation layer. The encapsulation layer TFE may be formed by laminating one layer or a plurality of layers. The encapsulation layer TFE includes at least one insulation layer. The encapsulation layer TFE according to one or more embodiments may include at least one inorganic film (hereinafter, an encapsulation-inorganic film). The encapsulation layer TFE according to one or more embodiments may also include at least one organic film (hereinafter, an encapsulation-organic film) and at least one encapsulation-inorganic film.

The encapsulation-inorganic film protects the display device layer DP-ED from moisture and/or oxygen, and the encapsulation-organic film protects the display device layer DP-ED from foreign substances such as dust particles. The encapsulation-inorganic film may include silicon nitride, silicon oxynitride, silicon oxide, titanium oxide, aluminum oxide, and/or the like, but the embodiment of the disclosure is not particularly limited thereto. The encapsulation-organic film may include an acrylic-based compound, an epoxy-based compound, and/or the like. The encapsulation-organic film may include a photopolymerizable organic material, but the embodiment of the disclosure is not particularly limited thereto.

The encapsulation layer TFE may be arranged on the second electrode EL2 and may be arranged filling the opening OH.

Referring to FIGS. 3 and 4, the display device DD may include a non-light emitting region NPXA and light emitting regions PXA-R, PXA-G, and PXA-B. The light emitting regions PXA-R, PXA-G, and PXA-B may be regions in which light generated by the respective light emitting elements ED-1, ED-2, and ED-3 is emitted. The light emitting regions PXA-R, PXA-G, and PXA-B may be spaced and/or apart (e.g., spaced apart or separated) from each other on a plane.

Each of the light emitting regions PXA-R, PXA-G, and PXA-B may be a region divided (e.g., defined) by the pixel defining film PDL. The non-light emitting regions NPXA may be regions between the adjacent light emitting regions PXA-R, PXA-G, and PXA-B, which correspond to the pixel defining film PDL. In one or more embodiments, the light emitting regions PXA-R, PXA-G, and PXA-B may respectively correspond to pixels. The pixel defining film PDL may divide light emitting elements ED-1, ED-2, and ED-3. The emission layers EML-R, EML-G, and EML-B of the light emitting elements ED-1, ED-2, and ED-3 may be arranged in openings OH defined in the pixel defining film PDL and separated from each other.

The light emitting regions PXA-R, PXA-G, and PXA-B may be divided (e.g., defined) into a plurality of groups according to the color of light generated from the light emitting elements ED-1, ED-2, and ED-3. In the display device DD of one or more embodiments illustrated in FIGS. 3 and 4, three light emitting regions PXA-R, PXA-G, and PXA-B, which emit red light, green light, and blue light, respectively, are exemplarily illustrated. For example, the display device DD of one or more embodiments may include the red light emitting region PXA-R, the green light emitting region PXA-G, and the blue light emitting region PXA-B that are separated from each other.

In the display device DD according to one or more embodiments, the plurality of light emitting elements ED-1, ED-2 and ED-3 may be to emit light beams having wavelengths different from each other. For example, in one or more embodiments, the display device DD may include a first light emitting element ED-1 that emits red light, a second light emitting element ED-2 that emits green light, and a third light emitting element ED-3 that emits blue light. For example, the red light emitting region PXA-R, the green light emitting region PXA-G, and the blue light emitting region PXA-B of the display device DD may correspond to the first light emitting element ED-1, the second light emitting element ED-2, and the third light emitting element ED-3, respectively.

However, the embodiment of the disclosure is not limited thereto, and the first to third light emitting elements ED-1, ED-2, and ED-3 may be to emit light beams in substantially the same wavelength range or at least one light emitting element may be to emit a light beam in a wavelength range different from the others. For example, the first to third light emitting elements ED-1, ED-2, and ED-3 may all emit blue light.

The light emitting regions PXA-R, PXA-G, and PXA-B in the display device DD according to one or more embodiments may be arranged in a stripe form. Referring to FIG. 3, the plurality of red light emitting regions PXA-R, the plurality of green light emitting regions PXA-G, and the plurality of blue light emitting regions PXA-B each may be arranged along a second directional axis DR2. In some embodiments, the red light emitting region PXA-R, the green light emitting region PXA-G, and the blue light emitting region PXA-B may be alternately arranged in this order along a first directional axis DR1.

FIGS. 3 and 4 illustrate that all the light emitting regions PXA-R, PXA-G, and PXA-B have similar area, but the embodiment of the disclosure is not limited thereto. Thus, the light emitting regions PXA-R, PXA-G, and PXA-B may have different areas from each other according to the wavelength range of the emitted light. In this case, the areas of the light emitting regions PXA-R, PXA-G, and PXA-B may refer to areas if (e.g., when) viewed on a plane defined by the first directional axis DR1 and the second directional axis DR2. A third directional axis DR3 may be perpendicular to a plane defined by the first directional axis DR1 and the second directional axis DR2.

In one or more embodiments, an arrangement form of the light emitting regions PXA-R, PXA-G, and PXA-B is not limited to the configuration illustrated in FIG. 1, and the order in which the red light emitting region PXA-R, the green light emitting region PXA-G, and the blue light emitting region PXA-B are arranged may be provided in one or more suitable combinations according to the characteristics of display quality desired or required in the display device DD. For example, the arrangement form of the light emitting regions PXA-R, PXA-G, and PXA-B may be a pentile (PENTILE®) arrangement form or a diamond (DIAMOND PIXEL™) arrangement form.

For example, the arrangement of the red light emitting region PXA-R, the green light emitting region PXA-G, and the blue light emitting region PXA-B may be arranged in a PENTILE® form or structure, (e.g., an RGBG matrix, an RGBG structure, or an RGBG matrix structure), for example, a DIAMOND PIXEL™ form or structure, e.g., a display (e.g., an OLED display) containing red, green, and blue (RGB) light emitting regions arranged in the shape of diamonds. PENTILE® and DIAMOND PIXEL™ are trademarks owned by Samsung Display Co., Ltd. However, the disclosure is not limited thereto.

In some embodiments, the areas of the light emitting regions PXA-R, PXA-G, and PXA-B may be different from each other. For example, in one or more embodiments, the area of the green light emitting region PXA-G may be smaller than that of the blue light emitting region PXA-B, but the embodiment of the disclosure is not limited thereto.

Hereinafter, FIGS. 5 to 8 are cross-sectional views schematically showing a light emitting element according to one or more embodiments. The light emitting element ED according to one or more embodiments may include a first electrode EL1, a second electrode EL2 opposite to (e.g., facing) the first electrode EL1, and at least one functional layer arranged between the first electrode EL1 and the second electrode EL2. The light emitting element ED according to one or more embodiments may include a nitrogen-containing compound according to one or more embodiments, which will be described later, in at least one functional layer.

The light emitting element ED may include a hole transport region HTR, an emission layer EML, and an electron transport region ETR, which are sequentially stacked, as at least one functional layer. For example, the light emitting element ED of one or more embodiments may include a first electrode EL1, a hole transport region HTR, an emission layer EML, an electron transport region ETR, and a second electrode EL2.

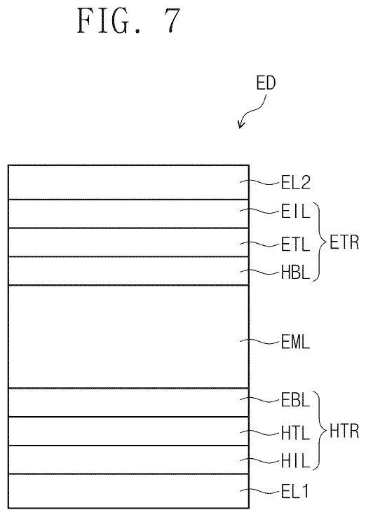

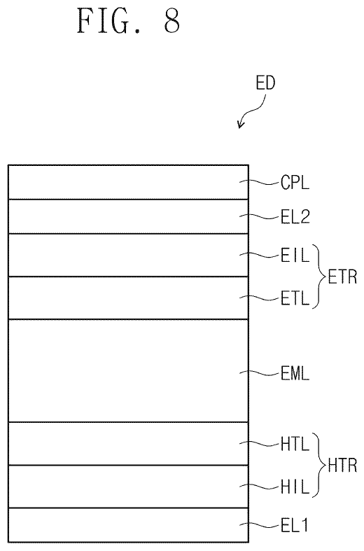

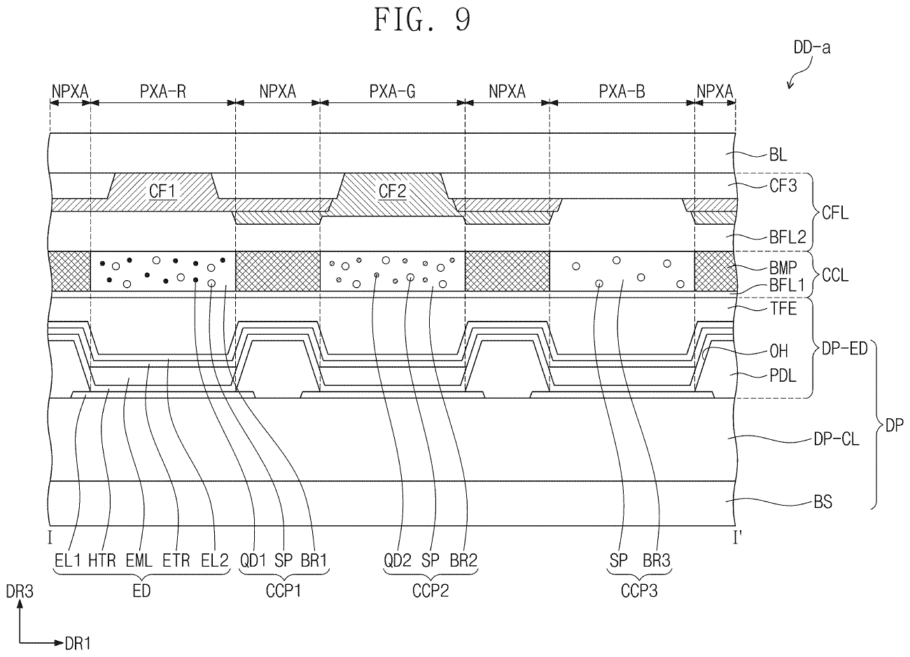

Compared with FIG. 5, FIG. 6 illustrates a cross-sectional view of a light emitting element ED of one or more embodiments, in which a hole transport region HTR includes a hole injection layer HIL and a hole transport layer HTL, and an electron transport region ETR includes an electron injection layer EIL and an electron transport layer ETL. In some embodiments, compared with FIG. 5, FIG. 7 illustrates a cross-sectional view of a light emitting element ED of one or more embodiments, in which a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. Compared with FIG. 6, FIG. 8 illustrates a cross-sectional view of a light emitting element ED of one or more embodiments including a capping layer CPL arranged on a second electrode EL2.

The light emitting element ED of one or more embodiments may include a nitrogen-containing compound of one or more embodiments, which will be described later, in at least one functional layer included in the light emitting element ED. For example, the emission layer EML may include the nitrogen-containing compound of one or more embodiments. However, the embodiment is not limited thereto.

The first electrode EL1 has conductivity (e.g., is a conductor). The first electrode EL1 may be formed of a metal material, a metal alloy, or a conductive compound. The first electrode EL1 may be an anode or a cathode. However, the embodiment of the disclosure is not limited thereto. In some embodiments, the first electrode EL1 may be a pixel electrode. The first electrode EL1 may be a transmissive electrode, a transflective electrode, or a reflective electrode. The first electrode EL1 may include at least one selected from among Ag, Mg, Cu, Al, Pt, Pd, Au, Ni, Nd, Ir, Cr, Li, Ca, LiF, Mo, Ti, W, In, Sn, and Zn, a compound of two or more selected from among these, a mixture of two or more selected from among these, or an oxide thereof.

If the first electrode EL1 is the transmissive electrode, the first electrode EL1 may include a transparent metal oxide such as indium tin oxide (ITO), indium zinc oxide (IZO), zinc oxide (ZnO), or indium tin zinc oxide (ITZO). If the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (a stacked structure of LiF and Ca), LiF/Al (a stacked structure of LiF and Al), Mo, Ti, W, a compound or mixture thereof (e.g., a mixture of Ag and Mg). In one or more embodiments, the first electrode EL1 may have a multilayer structure including a reflective film or a transflective film formed of the herein-described materials, and a transparent conductive film formed of ITO, IZO, ZnO, ITZO, and/or the like. For example, the first electrode EL1 may have a three-layer structure of ITO/Ag/ITO, but the embodiment of the disclosure is not limited thereto. In some embodiments, the embodiment of the disclosure is not limited thereto, and the first electrode EL1 may include the herein-described metal materials, combinations of at least two metal materials of the herein-described metal materials, oxides of the herein-described metal materials, and/or the like. The thickness of the first electrode EL1 may be from about 700 Å to about 10,000 Å. For example, the thickness of the first electrode EL1 may be from about 1,000 Å to about 3,000 Å.

The hole transport region HTR is provided on the first electrode EL1. The hole transport region HTR may include at least one of a hole injection layer HIL, a hole transport layer HTL, a buffer layer or an emission-auxiliary layer, or an electron blocking layer EBL. The thickness of the hole transport region HTR may be, for example, from about 50 Å to about 15,000 Å.

The hole transport region HTR may have a single layer formed of a single material, a single layer formed of a plurality of different materials, or a multilayer structure including a plurality of layers formed of a plurality of different materials.

For example, the hole transport region HTR may have a single layer structure of the hole injection layer HIL or the hole transport layer HTL, or may have a single layer structure formed of a hole injection material and a hole transport material. In some embodiments, the hole transport region HTR may have a single layer structure formed of a plurality of different materials, or a structure in which a hole injection layer HIL/hole transport layer HTL, a hole injection layer HIL/hole transport layer HTL/buffer layer, a hole injection layer HIL/buffer layer, a hole transport layer HTL/buffer layer, or a hole injection layer HIL/hole transport layer HTL/electron blocking layer EBL are stacked in order from the first electrode EL1, but the embodiment of the disclosure is not limited thereto.

The hole transport region HTR may be formed using one or more suitable methods such as a vacuum deposition method, a spin coating method, a cast method, a Langmuir-Blodgett (LB) method, an inkjet printing method, a laser printing method, and a laser induced thermal imaging (LITI) method.

The hole transport region HTR may include a compound represented by Formula H-1:

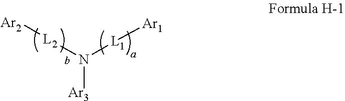

In Formula H-1, L₁ and L₂ may each independently be a direct linkage, a substituted or unsubstituted arylene group having 6 to 30 ring-forming carbon atoms, or a substituted or unsubstituted heteroarylene group having 2 to 30 ring-forming carbon atoms. a and b may each independently be an integer of 0 to 10. In one or more embodiments, if (e.g., when) a or b is an integer of 2 or greater, a plurality of L₁'s and L₂'s may each independently be a substituted or unsubstituted arylene group having 6 to 30 ring-forming carbon atoms, or a substituted or unsubstituted heteroarylene group having 2 to 30 ring-forming carbon atoms.

In Formula H-1, Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. In some embodiments, in Formula H-1, Ar₃ may be a substituted or unsubstituted aryl group having 6 to 30 ring-forming carbon atoms, or a substituted or unsubstituted heteroaryl group having 2 to 30 ring-forming carbon atoms.

The compound represented by Formula H-1 may be a monoamine compound. In one or more embodiments, the compound represented by Formula H-1 may be a diamine compound in which at least one among Ar₁ to Ar₃ includes the amine group as a substituent. In some embodiments, the compound represented by Formula H-1 may be a carbazole-based compound including a substituted or unsubstituted carbazole group in at least one of Ar₁ or Ar₂, or a fluorene-based compound including a substituted or unsubstituted fluorene group in at least one of Ar₁ or Ar₂.

The compound represented by Formula H-1 may be represented by any one among (e.g., any one selected from among) the compounds in Compound Group H. However, the compounds listed in Compound Group H are examples, and the compounds represented by Formula H-1 are not limited to those represented by Compound Group H:

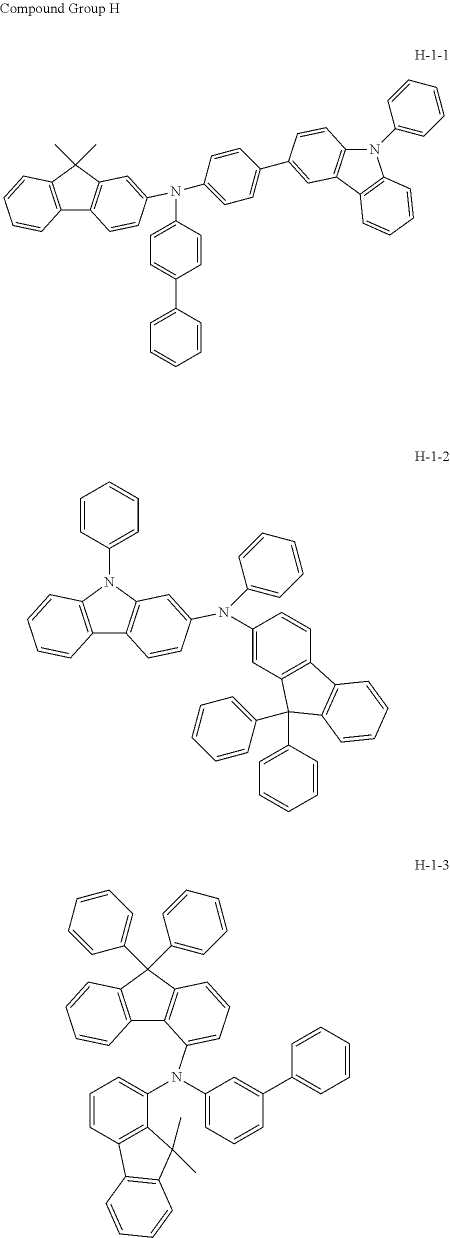 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21]

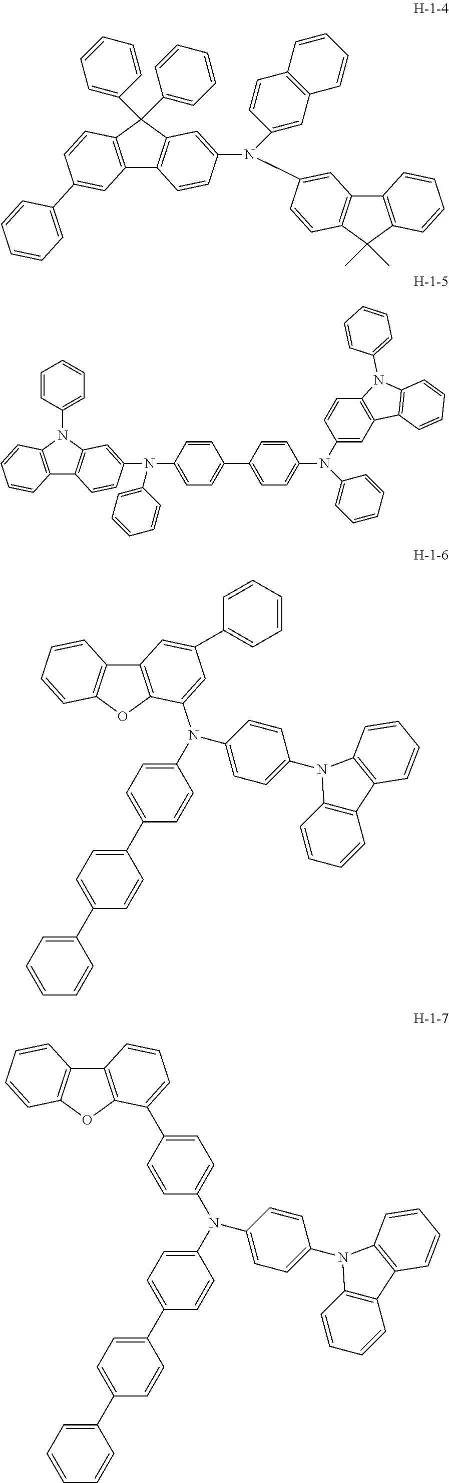 [SMILES: CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21 ; c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

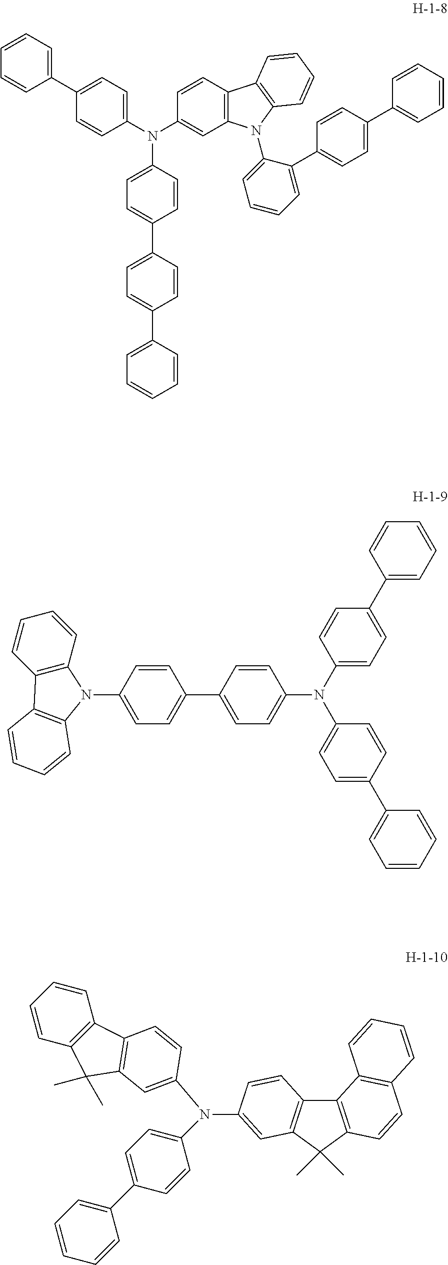 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

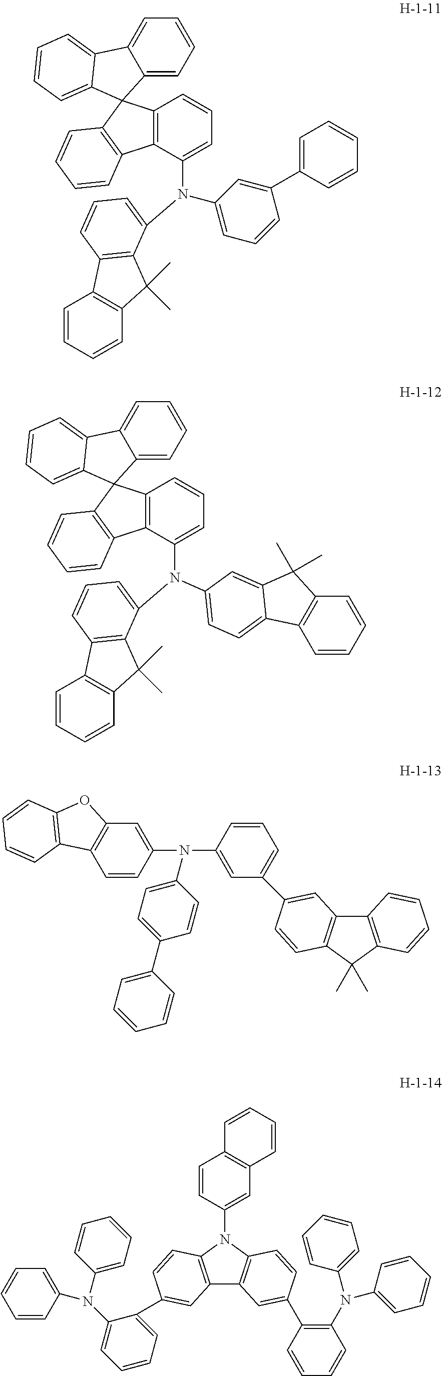 [SMILES: CC1(C)c2ccccc2-c2cccc(N(c3cccc(-c4ccccc4)c3)c3cccc4c3-c3ccccc3C43c4ccccc4-c4ccccc43)c21 ; CC1(C)c2ccccc2-c2ccc(N(c3cccc4c3-c3ccccc3C43c4ccccc4-c4ccccc43)c3cccc4c3C(C)(C)c3ccccc3-4)cc21 ; CC1(C)c2ccccc2-c2cc(-c3cccc(N(c4ccc(-c5ccccc5)cc4)c4ccc5c(c4)oc4ccccc45)c3)ccc21 ; c1ccc(N(c2ccccc2)c2ccccc2-c2ccc3c(c2)c2cc(-c4ccccc4N(c4ccccc4)c4ccccc4)ccc2n3-c2ccc3ccccc3c2)cc1]

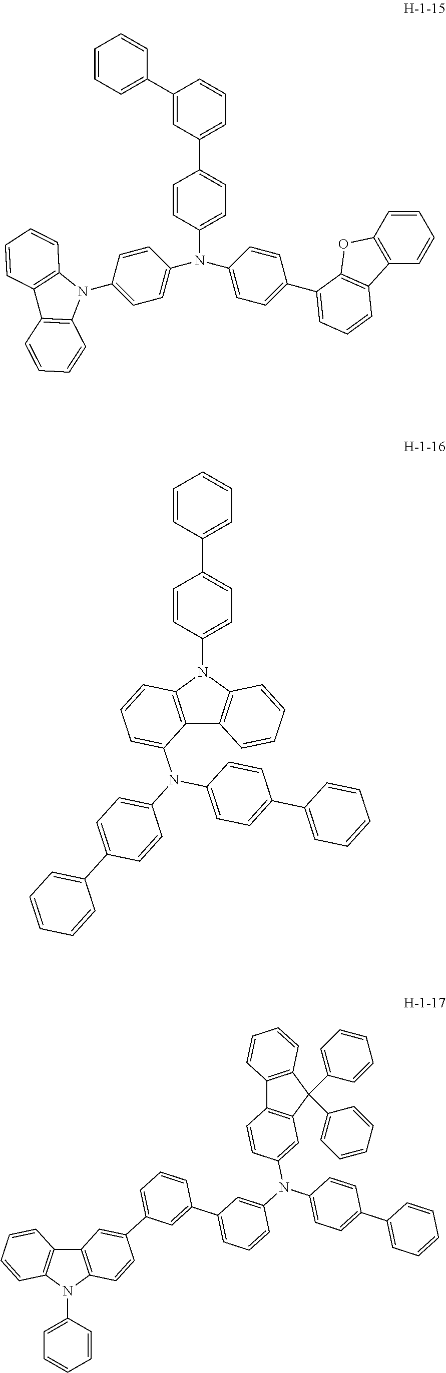 [SMILES: c1ccc(-c2cccc(-c3ccc(N(c4ccc(-c5cccc6c5oc5ccccc56)cc4)c4ccc(-n5c6ccccc6c6ccccc65)cc4)cc3)c2)cc1 ; c1ccc(-c2ccc(N(c3ccc(-c4ccccc4)cc3)c3cccc4c3c3ccccc3n4-c3ccc(-c4ccccc4)cc3)cc2)cc1 ; c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1]

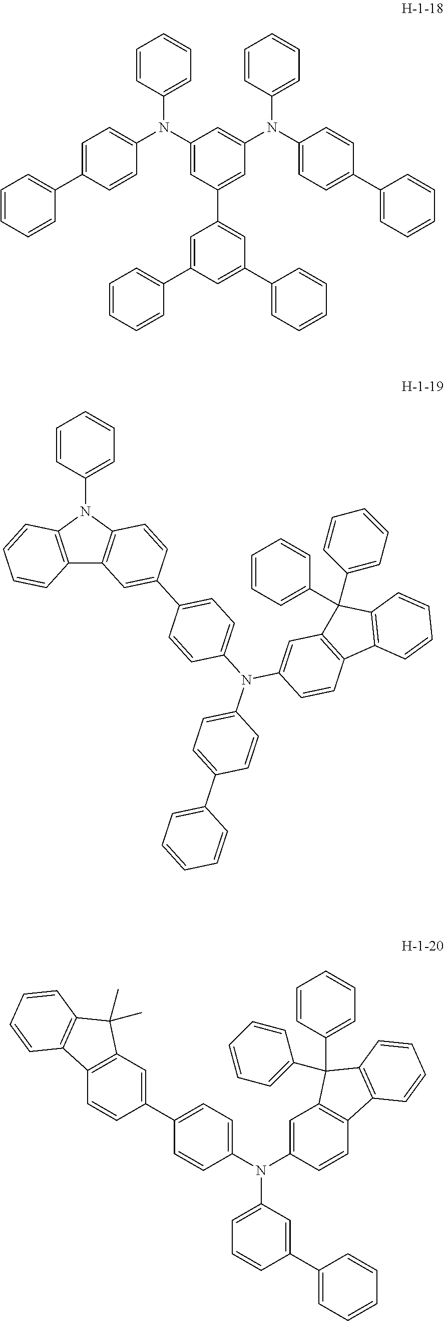 [SMILES: c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1 ; CC1(C)c2ccccc2-c2ccc(-c3ccc(N(c4cccc(-c5ccccc5)c4)c4ccc5c(c4)C(c4ccccc4)(c4ccccc4)c4ccccc4-5)cc3)cc21]

The hole transport region HTR may include a phthalocyanine compound such as copper phthalocyanine, N¹,N^{1′}-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino]triphenylamine (m-MTDATA), 4,4′,4″-tris(N,N-diphenylamino)triphenylamine (TDATA), 4,4′,4″-tris[N-(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-1-yl)-N,N′-diphenyl-benzidine (NPB), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HAT-CN), and/or the like.

The hole transport region HTR may include a carbazole-based derivative such as N-phenyl carbazole or polyvinyl carbazole, a fluorene-based derivative, a triphenylamine-based derivative such as N,N′-bis(3-methylphenyl)-N,N′-diphenyl-[1,1-biphenyl]-4,4′-diamine (TPD) or 4,4′,4″-tris(N-carbazolyl)triphenylamine (TCTA), N,N′-di(naphthalene-1-yl)-N,N′-diphenyl-benzidine (NPB), 4,4′-cyclohexylidene bis[N,N-bis(4-methylphenyl]benzenamine](TAPC), 4,4′-bis[N,N′-(3-tolyl)amino]-3,3′-dimethylbiphenyl (HMTPD), 1,3-bis(N-carbazolyl)benzene (mCP), and/or the like.

In some embodiments, the hole transport region HTR may include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), and/or the like.

The hole transport region HTR may include the herein-described compounds of the hole transport region HTR in at least one of a hole injection layer HIL, a hole transport layer HTL, or an electron blocking layer EBL.

The thickness of the hole transport region HTR may be from about 100 Å to about 10,000 Å, for example, from about 100 Å to about 5,000 Å. When the hole transport region HTR includes the hole injection layer HIL, the hole injection layer HIL may have, for example, a thickness of about 30 Å to about 1,000 Å. When the hole transport region HTR includes the hole transport layer HTL, the hole transport layer HTL may have a thickness of about 250 Å to about 1,000 Å. For example, if (e.g., when) the hole transport region HTR includes the electron blocking layer EBL, the electron blocking layer EBL may have a thickness of about 10 Å to about 1,000 Å. If the thicknesses of the hole transport region HTR, the hole injection layer HIL, the hole transport layer HTL and the electron blocking layer EBL satisfy the herein-described ranges, satisfactory hole transport properties may be achieved without a substantial increase in driving voltage.

The hole transport region HTR may further include a charge generating material to increase conductivity in addition to the herein-described materials. The charge generating material may be dispersed uniformly (e.g., substantially uniformly) or non-uniformly (e.g., substantially non-uniformly) in the hole transport region HTR. The charge generating material may be, for example, a p-dopant. The p-dopant may include at least one of a halogenated metal compound, a quinone derivative, a metal oxide, or a cyano group-containing compound, but the embodiment of the disclosure is not limited thereto. For example, the p-dopant may include a metal halide compound such as CuI or RbI, a quinone derivative such as tetracyanoquinodimethane (TCNQ) or 2,3,5,6-tetrafluoro-7,7′8,8-tetracyanoquinodimethane (F4-TCNQ), a metal oxide such as tungsten oxide or molybdenum oxide, a cyano group-containing compound such as dipyrazino[2,3-f: 2′,3′-h]quinoxaline-2,3,6,7,10,11-hexacarbonitrile (HATCN) or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), and/or the like, but the embodiment of the disclosure is not limited thereto.

As described, the hole transport region HTR may further include at least one of the buffer layer or the electron blocking layer EBL in addition to the hole injection layer HIL and the hole transport layer HTL. The buffer layer may compensate for a resonance distance according to the wavelength of light emitted from the emission layer EML and may thus increase light emission efficiency. A material that may be included in the hole transport region HTR may be used as a material to be included in the buffer layer. The electron blocking layer EBL is a layer that serves to prevent or reduce the electron injection from the electron transport region ETR to the hole transport region HTR.

The emission layer EML is provided on the hole transport region HTR. The emission layer EML may have a thickness of, for example, about 100 Å to about 1,000 Å or about 100 Å to about 300 Å. The emission layer EML may have a single layer formed of a single material, a single layer formed of a plurality of different materials, or a multilayer structure having a plurality of layers formed of a plurality of different materials.

The light emitting element ED of one or more embodiments may include the nitrogen-containing compound of one or more embodiments in the emission layer EML arranged between a first electrode EL1 and a second electrode EL2. The nitrogen-containing compound of one or more embodiments may be used as a host material of the emission layer EML. The emission layer EML may include the nitrogen-containing compound as an electron transporting host. In one or more embodiments, herein, the nitrogen-containing compound of one or more embodiments may be referred to as a first compound.

### Nitrogen-Containing Compound

The nitrogen-containing compound of one or more embodiments may include a core structure of a six-membered ring containing three heteroatoms and three carbon atoms. In the nitrogen-containing compound of one or more embodiments, the core may be a heterocycle having three ring-forming carbon atoms, containing three heteroatoms selected from among a nitrogen atom, an oxygen atom, and/or a sulfur atom as ring-forming atoms. The heterocycle of the core may be substituted or unsubstituted, and may include at least one nitrogen atom as a ring-forming atom.

The nitrogen-containing compound of one or more embodiments may include two heterocycle substituents, each of which is connected to the core via a linker. For example, the heterocycle substituent may be a benzothiazole derivative, a benzoxazole derivative, a benzimidazole derivative, a dihydrobenzothiazole derivative, a dihydrobenzoxazole derivative, or a dihydrobenzimidazole derivative. The two heterocycle substituents included in the nitrogen-containing compound of one or more embodiments may be the same or different. In the nitrogen-containing compound of one or more embodiments, the linker may be a substituted or unsubstituted heteroarylene group having 6 or more ring-forming carbon atoms, and containing at least one nitrogen atom as a ring-forming atom.

The nitrogen-containing compound of one or more embodiments may be represented by Formula 1. In the nitrogen-containing compound of one or more embodiments represented by Formula 1, ring A may correspond to the core described herein. In some embodiments, R₂ and R₃ may each correspond to the heterocycle substituent described herein, and may be represented by either Formula S-1 or Formula S-2. In Formula 1, L₁ and L₂ may correspond to the linker described herein. The heterocycle substituents of R₂ and R₃ may be connected to the core through the linker of L₁ and L₂.

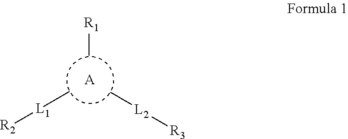

In Formula 1, ring A may be a substituted or unsubstituted heterocycle having 3 ring-forming carbon atoms, containing three heteroatoms selected from among a nitrogen atom, an oxygen atom, and a sulfur atom as ring-forming atoms. Ring A may include at least one nitrogen atom as a ring-forming atom.

For example, ring A may be an aliphatic heterocycle containing three heteroatoms selected from among N, O, and S as ring-forming atoms, and containing at least one N as a heteroatom. In one or more embodiments, ring A may be an aromatic heterocycle containing three heteroatoms selected from among N, O, and S as ring-forming atoms, and containing at least one N as a heteroatom. The three heteroatoms included in ring A may be the same or different. Ring A may include three N's as heteroatoms. The embodiment of the disclosure is not limited thereto, and ring A may contain one or two N's as heteroatoms, and may contain O or S as the other heteroatoms other than N.

In Formula 1, L₁ and L₂ may each independently be a substituted or unsubstituted heteroarylene group having 6 to 30 ring-forming carbon atoms. In one or more embodiments, L₁ and L₂ may each independently be a substituted or unsubstituted heteroarylene group having 12 to 30 ring-forming carbon atoms. In one or more embodiments, L₁ and L₂ of the heteroarylene group may contain at least one nitrogen atom as a ring-forming atom. L₁ and L₂ may be the same or different. In one or more embodiments, L₁ and L₂ may each independently be a substituted or unsubstituted divalent carbazole derivative. For example, L₁ and L₂ may each independently be a divalent carbazole derivative, such as a substituted or unsubstituted divalent carbazole group, a substituted or unsubstituted divalent indolocarbazole group, a substituted or unsubstituted divalent benzofurocarbazole group, or a substituted or unsubstituted divalent benzothienocarbazole group.

In Formula 1, R₁ may be a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms. In one or more embodiments, R₁ may be a substituted or unsubstituted aryl group having 6 to 60 ring-forming carbon atoms. For example, R₁ may be a substituted or unsubstituted phenyl group, but the embodiment is not limited thereto.

In Formula 1, R₂ and R₃ may each independently be represented by Formula S-1 or Formula S-2. Both R₂ and R₃ in Formula 1 may be represented by Formula S-1 or Formula S-2. Any one of R₂ and R₃ may be represented by Formula S-1 and the other may be represented by Formula S-2. R₂ and R₃ may be the same or different.

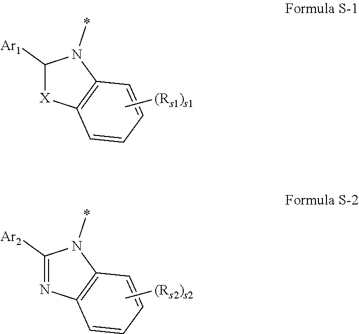

In Formula S-1, X may be NRₛ₃, O, or S. For example, X may be O or S, but the embodiment is not limited thereto.

In Formulas S-1 and S-2, Ar₁ and Ar₂ may each independently be a substituted or unsubstituted aryl group having 6 to 60 ring-forming carbon atoms, or a substituted or unsubstituted heteroaryl group having 2 to 60 ring-forming carbon atoms. Ar₁ and Ar₂ may each independently be a substituted or unsubstituted aryl group having 6 to 30 ring-forming carbon atoms. For example, Ar₁ and Ar₂ may each independently be a substituted or unsubstituted phenyl group.

In Formulas S-1 and S-2, Rₛ₁ to Rₛ₃ may each independently be a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms. For example, Rₛ₁ to Rₛ₃ may be hydrogen atoms, but the embodiment is not limited thereto.

In Formulas S-1 and S-2, s1 and s2 may each independently be an integer of 0 to 4. When s1 and s2 are each 0, the nitrogen-containing compound of one or more embodiments may not be substituted with each of Rₛ₁ and Rₛ₂. In Formulas S-1 and S-2, if (e.g., when) s1 and s2 are each 4 and each of Rₛ₁ and Rₛ₂ is all hydrogen atoms, the case may be the same as if (e.g., when) s1 and s2 are each 0 in Formulas S-1 and S-2. When s1 and s2 are each an integer of 2 or more, each of Rₛ₁ and Rₛ₂ provided in plurality may all be the same, or at least one of Rₛ₁ or Rₛ₂ provided in plurality may be different.

In Formulas S-1 and S-2, [symbol] is a site linked to Formula 1.

In one or more embodiments, the nitrogen-containing compound represented by Formula 1 may be represented by Formula 2-1 or Formula 2-2. The nitrogen-containing compound represented by Formula 2-1 may correspond to the case where both (e.g., simultaneously) R₂ and R₃ in Formula 1 are represented by Formula S-1. The nitrogen-containing compound represented by Formula 2-2 may correspond to the case where both (e.g., simultaneously) R₂ and R₃ in Formula 1 are represented by Formula S-2.

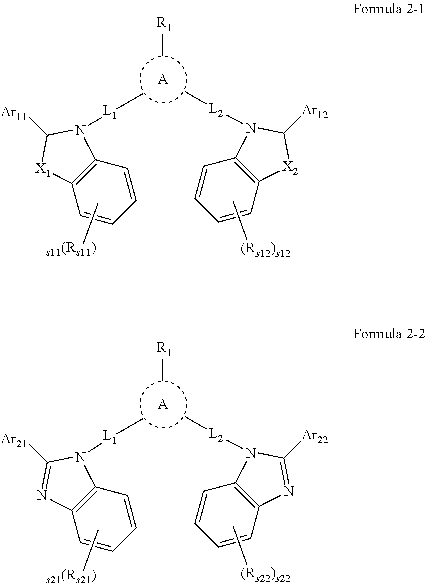

In Formula 2-1, X₁ and X₂ may each independently be O or S. X₁ and X₂ may be the same or different. For example, both (e.g., simultaneously) X₁ and X₂ may be O, or both (e.g., simultaneously) X₁ and X₂ may be S. The embodiment of the disclosure is not limited thereto, and one of X₁ and X₂ may be O, and the other may be S.

In Formulas 2-1 and 2-2, Ar₁₁, Ar₁₂, Ar₂₁, and Ar₂₂ may each independently be a substituted or unsubstituted aryl group having 6 to 30 ring-forming carbon atoms. For example, Ar₁₁, Ar₁₂, Ar₂l, and Ar₂₂ may each independently be a substituted or unsubstituted phenyl group.

In Formulas 2-1 and 2-2, Rₛ₁₁, Rₛ₁₂, Rₛ₂₁, and Rₛ₂₂ may each independently be a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms. For example, Rₛ₁₁, Rₛ₁₂, Rₛ₂₁, and Rₛ₂₂ may be hydrogen atoms, but the embodiment is not limited thereto.

In Formulas 2-1 and 2-2, s11, s12, s21, and s22 may each independently be an integer of 0 to 4. When s11, s12, s21, and s22 are each 0, the nitrogen-containing compound of one or more embodiments may not be substituted with each of Rₛ₁₁, Rₛ₁₂, Rₛ₂₁, and Rₛ₂₂. In Formulas 2-1 and 2-2, if (e.g., when) s11, s12, s21, and s22 are each 4, and each of Rₛ₁₁, Rₛ₁₂, Rₛ₂₁, and Rₛ₂₂ is all hydrogen atoms, the case may be the same as if (e.g., when) s11, s12, s21, and s22 are each 0 in Formulas 2-1 and 2-2. When s11, s12, s21, and s22 are each an integer of 2 or more, each of Rₛ₁₁, Rₛ₁₂, Rₛ₂₁, and Rₛ₂₂ provided in plurality may all be the same, or at least one of Rₛ₁₁, Rₛ₁₂, Rₛ₂₁, or Rₛ₂₂ provided in plurality may be different. In Formulas 2-1 and 2-2, the descriptions as in Formula 1 may apply to ring A, L₁, L₂, and R₁.

In one or more embodiments, the nitrogen-containing compound represented by Formula 1 may be represented by Formula 3-1 or Formula 3-2. Formulas 3-1 and 3-2 may correspond to the case where L₁ and L₂ of Formula 1 are divalent carbazole derivatives.

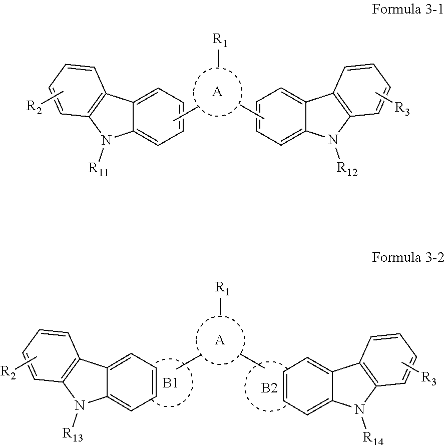

In Formula 3-2, ring B1 and ring B2 may each independently be a substituted or unsubstituted hydrocarbon ring having 5 to 30 ring-forming carbon atoms, or a substituted or unsubstituted heterocycle having 2 to 30 ring-forming carbon atoms. The number of ring B1's and ring B2's may each be 1. Ring B1 and ring B2 may form a fused ring with one hydrocarbon ring or heterocycle at portions indicated by B1 and B2. Ring B1 and ring B2 may be the same or different.

In Formulas 3-1 and 3-2, R₁₁, R₁₂, R₁₃, and R₁₄ may each independently be a substituted or unsubstituted aryl group having 6 to 30 ring-forming carbon atoms. For example, R₁₁, R₁₂, R₁₃, and R₁₄ may be substituted or unsubstituted phenyl groups.

In Formulas 3-1 and 3-2, the descriptions as in Formula 1 may apply to ring A, R₁, R₂, and R₃.

The nitrogen-containing compound of one or more embodiments may be represented by Formula 3-1-1 or Formula 3-1-2. The nitrogen-containing compound represented by Formula 3-1-1 and the nitrogen-containing compound represented by Formula 3-1-2 may correspond to the case where R₂ and R₃ in Formula 3-1 are represented by Formula S-1 or Formula S-2. In some embodiments, the nitrogen-containing compound represented by Formula 3-1-1 and the nitrogen-containing compound represented by Formula 3-1-2 may each correspond to the case where in Formula 1, the linkers of L₁ and L₂ are divalent carbazole derivatives and the heterocycle substituents of R₂ and R₃ are represented by Formula S-1 or Formula S-2.

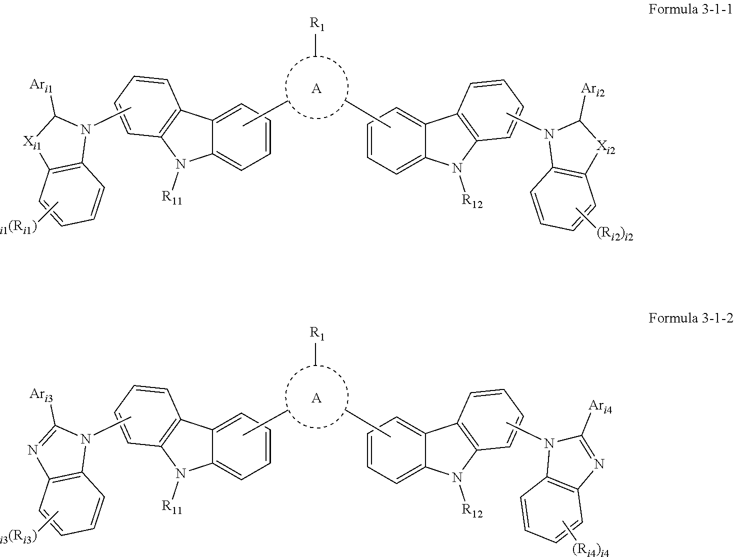

In Formula 3-1-1, Xᵢ₁ and X₁₂ may each independently be O or S. Xᵢ₁ and Xᵢ₂ may be the same or different. For example, both (e.g., simultaneously) Xᵢ₁ and Xᵢ₂ may be O, or both (e.g., simultaneously) Xᵢ₁ and Xᵢ₂ may be S. The embodiment of the disclosure is not limited thereto, and one of Xᵢ₁ and Xᵢ₂ may be O, and the other may be S.

In Formulas 3-1-1 and 3-1-2, Arᵢ₁, Arᵢ₂, Arᵢ₃₁, and Arᵢ₄ may each independently be a substituted or unsubstituted aryl group having 6 to 30 ring-forming carbon atoms. For example, Arᵢ₁, Arᵢ₂, Arᵢ₃₁, and Arᵢ₄ may each independently be a substituted or unsubstituted phenyl group.

In Formulas 3-1-1 and 3-1-2, Rᵢ₁, Rᵢ₂, Rᵢ₃, and Rᵢ₄ may each independently be a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms. For example, Rᵢ₁, Rᵢ₂, Rᵢ₃, and Rᵢ₄ may be hydrogen atoms, but the embodiment is not limited thereto.

In Formulas 3-1-1 and 3-1-2, i1, i2, i3, and i4 may each independently be an integer of 0 to 4. When i1, i2, i3, and 4 are each 0, the nitrogen-containing compound of one or more embodiments may not be substituted with each of Rᵢ₁, Rᵢ₂, Rᵢ₃, and Rᵢ₄. In Formulas 3-1-1 and 3-1-2, if (e.g., when) i1, i2, i3, and i4 are each 4, and each of Rᵢ₁, Rᵢ₂, Rᵢ₃, and Rᵢ₄ is all hydrogen atoms, the case may be the same as if (e.g., when) i1, i2, i3, and i4 are each 0 in Formulas 3-1-1 and 3-1-2. When i1, i2, i3, and i4 are each an integer of 2 or more, each of Rᵢ₁, Rᵢ₂, Rᵢ₃, and Rᵢ₄ provided in plurality may all be the same, or at least one of Rᵢ₁, Rᵢ₂, Rᵢ₃, or Rᵢ₄ provided in plurality may be different.

In Formulas 3-1-1 and 3-1-2, the descriptions as in Formulas 1 and 3-1 may apply to ring A, R₁, R₁₁, and R₁₂.

The nitrogen-containing compound of one or more embodiments may be represented by Formula 3-2-1 or Formula 3-2-2. The nitrogen-containing compound represented by Formula 3-2-1 and the nitrogen-containing compound represented by Formula 3-2-2 may each correspond to the case where the heterocycle substituents of R₂ and R₃ in Formula 3-2 are represented by Formula S-1 or Formula S-2. In some embodiments, the nitrogen-containing compound represented by Formula 3-2-1 and the nitrogen-containing compound represented by Formula 3-2-2 may each correspond to the case where in Formula 1, the linkers of L₁ and L₂ are divalent carbazole group derivatives and the heterocycle substituents of R₂ and R₃ are represented by Formula S-1 or Formula S-2.

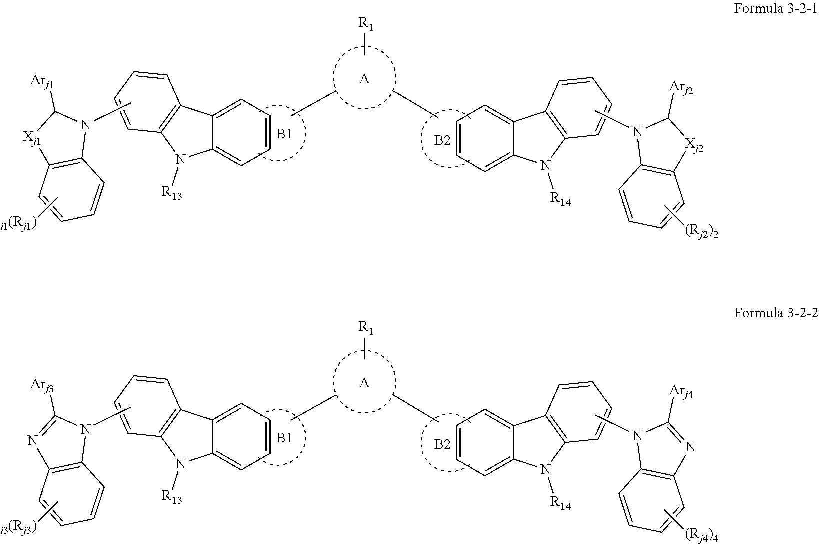

In Formula 3-2-1, Xⱼ₁ and Xⱼ₂ may each independently be O or S. Xⱼ₁ and Xⱼ₂ may be the same or different. For example, both (e.g., simultaneously) Xⱼ₁ and Xⱼ₂ may (each) be O, or both (e.g., simultaneously) Xⱼ₁ and Xⱼ₂ may (each) be S. The embodiment of the disclosure is not limited thereto, and one of Xⱼ₁ and Xⱼ₂ may be O, and the other may be S.

In Formulas 3-2-1 and 3-2-2, Arⱼ₁, Arⱼ₂, Arⱼ₃, and Arⱼ₄ may each independently be a substituted or unsubstituted aryl group having 6 to 30 ring-forming carbon atoms. For example, Arⱼ₁, Arⱼ₂, Arⱼ₃, and Arⱼ₄ may each independently be a substituted or unsubstituted phenyl group.

In Formulas 3-2-1 and 3-2-2, Rⱼ₁, Rⱼ₂, Rⱼ₃, and Rⱼ₄ may each independently be a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms. For example, Rⱼ₁, Rⱼ₂, Rⱼ₃, and Rⱼ₄ may be hydrogen atoms, but the embodiment is not limited thereto.

In Formulas 3-2-1 and 3-2-2, j1, j2, j3, and 4 may each independently be an integer of 0 to 4. When j1, j2, j3, and j4 are each 0, the nitrogen-containing compound of one or more embodiments may not be substituted with each of Rⱼ₁, Rⱼ₂, Rⱼ₃, and Rⱼ₄.

In Formulas 3-2-1 and 3-2-2, if (e.g., when) j1, j2, j3, and j4 are each 4, and each of Rⱼ₁, Rⱼ₂, Rⱼ₃, and Rⱼ₄ is all hydrogen atoms, the case may be the same as if (e.g., when) j1, j2, j3, and j4 are each 0 in Formulas 3-2-1 and 3-2-2. When j1, j2, j3, and j4 are each an integer of 2 or more, each of Rⱼ₁, Rⱼ₂, Rⱼ₃, and Rⱼ₄ provided in plurality may all be the same, or at least one of Rⱼ₁, Rⱼ₂, Rⱼ₃, or Rⱼ₄ provided in plurality may be different.

In Formulas 3-2-1 and 3-2-2, the descriptions as in Formulas 1 and 3-2 may apply to ring A, ring B1, ring B2, R₁, R₁₃, and R₁₄.

In one or more embodiments, the nitrogen-containing compound represented by Formula 1 may be represented by Formula 4 or Formula 5. The nitrogen-containing compound represented by Formula 4 may include an aromatic heterocycle containing three N's as a core. In Formula 4, a 6-membered ring containing three N's may correspond to ring A of Formula 1. The nitrogen-containing compound represented by Formula 5 may include an aliphatic heterocycle containing three heteroatoms selected from among N, O, and S as ring-forming atoms as a core, and containing at least one N as a heteroatom. In Formula 5, a six-membered ring containing Y₁, Y₂, and Y₃ may correspond to ring A of Formula 1.

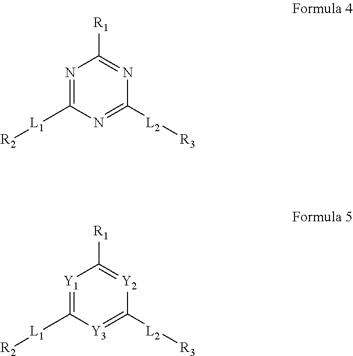

In Formula 5, at least one of Y₁ to Y₃ may be NR₄, and the others may each independently be O or S. For example, Y₁ to Y₃ may all be NR₄. The embodiment of the disclosure is not limited thereto, and one or two of Y₁ to Y₃ may be NR₄, and the others that are not NR₄ may each independently be O or S.

In Formula 5, R₄ may be a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms.

In Formulas 4 and 5, the descriptions as in Formula 1 may apply to L₁, L₂, and R₁ to R₃.

The nitrogen-containing compound of one or more embodiments may be represented by Formula 4-1 or Formula 4-2. The nitrogen-containing compound represented by Formulas 4-1 and 4-2 may correspond to the case where L₁ and L₂ in Formula 4 are divalent carbazole derivatives. In some embodiments, the nitrogen-containing compound represented by Formula 4-1 may correspond to the case where ring A in Formula 3-1 is limited to a six-membered ring containing three N's, and the nitrogen-containing compound represented by Formula 4-2 may correspond to the case where ring A in Formula 3-2 is limited to a six-membered ring containing three N's.

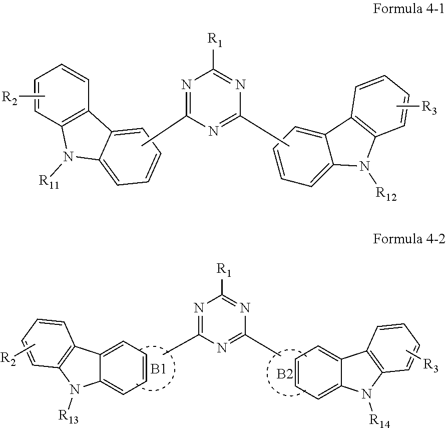

In Formulas 4-1 and 4-2, ring B1 and ring B2 may each independently be a substituted or unsubstituted hydrocarbon ring having 5 to 30 ring-forming carbon atoms, or a substituted or unsubstituted heterocycle having 2 to 30 ring-forming carbon atoms. The number of ring B1's and ring B2's may each be 1. Ring B1 and ring B2 may form a fused ring with one hydrocarbon ring or heterocycle at portions indicated by B1 and B2. Ring B1 and ring B2 may be the same or different.

In Formulas 4-1 and 4-2, R₁₁, R₁₂, R₁₃, and R₁₄ may each independently be a substituted or unsubstituted aryl group having 6 to 30 ring-forming carbon atoms. For example, R₁₁, R₁₂, R₁₃, and R₁₄ may be substituted or unsubstituted phenyl groups.

As described herein, in Formulas 4-1 and 4-2, ring B1, ring B2, R₁₁, R₁₂, R₁₃, and R₁₄ may be the same as in Formulas 3-1 and 3-2. In one or more embodiments, in Formulas 4-1 and 4-2, the descriptions as in Formula 1 may apply to R₁, R₂, and R₃.

The nitrogen-containing compound of one or more embodiments may be represented by Formula 4-1-1, Formula 4-1-2, Formula 4-2-1, or Formula 4-2-2. The nitrogen-containing compound represented by Formula 4-1-1 and the nitrogen-containing compound represented by Formula 4-1-2 may each correspond to the case where the heterocycle substituents of R₂ and R₃ in Formula 4-1 are represented by Formula S-1 or Formula S-2. In some embodiments, the nitrogen-containing compound represented by Formula 4-1-1 may correspond to the case where ring A in Formula 3-1-1 is limited to a six-membered ring containing three N's, and the nitrogen-containing compound represented by Formula 4-1-2 may correspond to the case where ring A in Formula 3-1-2 is limited to a six-membered ring containing three N's.

The nitrogen-containing compound represented by Formula 4-2-1 and the nitrogen-containing compound represented by Formula 4-2-2 may each correspond to the case where the heterocycle substituents of R₂ and R₃ in Formula 3-2 are represented by Formula S-1 or Formula S-2. In some embodiments, the nitrogen-containing compound represented by Formula 4-2-1 may correspond to the case where ring A in Formula 3-2-1 is limited to a six-membered ring containing three N's, and the nitrogen-containing compound represented by Formula 4-2-2 may correspond to the case where ring A in Formula 3-2-2 is limited to a six-membered ring containing three N's.

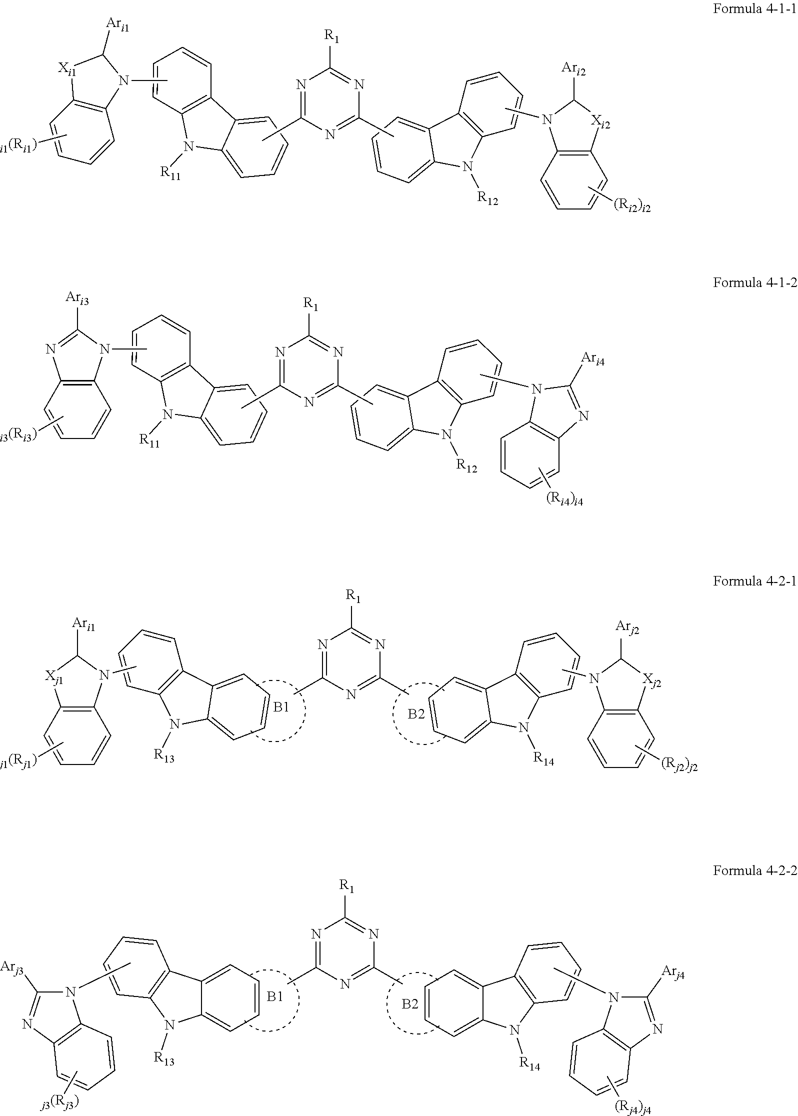

In Formulas 4-1-1 and 4-1-2, the descriptions as in Formulas 1, 3-1, 3-1-1, and 3-1-2 may apply to Xᵢ₁, Xᵢ₂, Arᵢ₁, Arᵢ₂, Arᵢ₃, Arᵢ₄, R₁, R₁₁, R₁₂, Rᵢ₁, Rᵢ₂, Rᵢ₃, Rᵢ₄, i1, i2, i3, and i4.

In Formulas 4-2-1 and 4-2-2, the descriptions as in Formulas 1, 3-2, 3-2-1, and 3-2-2 may apply to ring B1, ring B2, Xⱼ₁, Xⱼ₂, Arⱼ₁, Arⱼ₂, Arⱼ₃, Arⱼ₄, R₁, R₁₃, R₁₄, Rⱼ₁, Rⱼ₂, Rᵢ₃, Rⱼ₄, j1, j2, j3, and j4.

In one or more embodiments, the nitrogen-containing compound of one or more embodiments represented by Formula 1 may include at least one deuterium atom as a substituent. The nitrogen-containing compound of one or more embodiments represented by Formula 1 may include a structure in which at least one hydrogen atom is substituted with a deuterium atom.

The nitrogen-containing compound of one or more embodiments may be any one among (e.g., any one selected from among) the compounds shown in Compound Group 1. The light emitting element ED of one or more embodiments may include at least one nitrogen-containing compound among (e.g., at at least one nitrogen-containing compound selected from among) the compounds shown in Compound Group 1 in the emission layer EML.

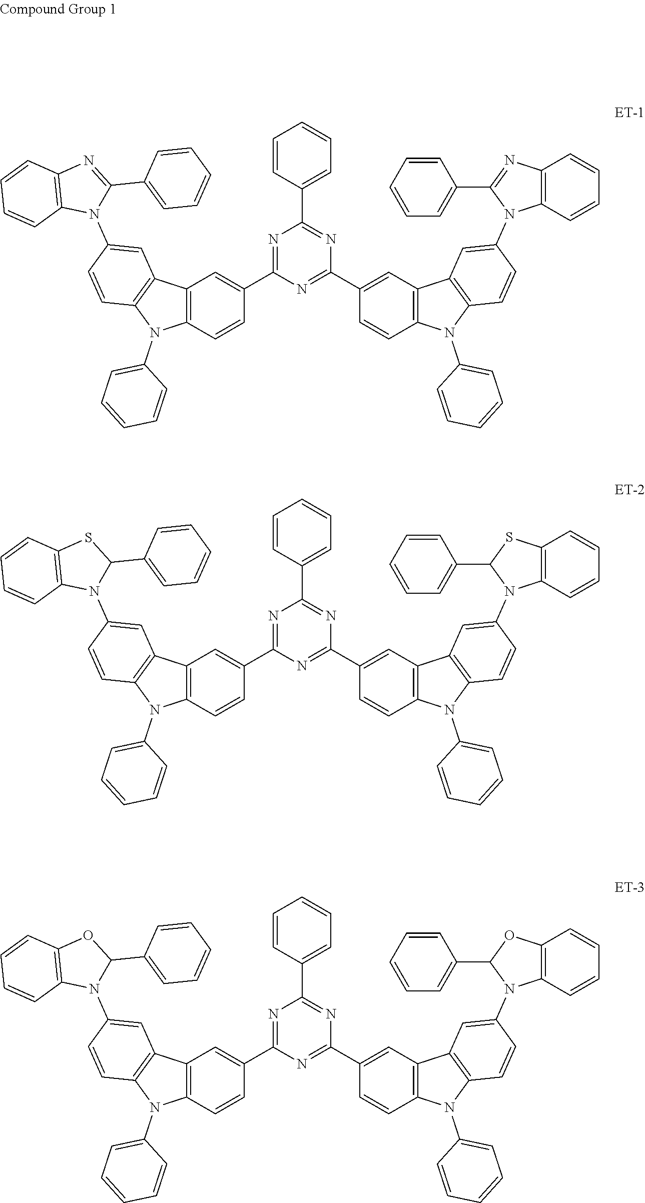 [SMILES: c1ccc(-c2nc(-c3ccc4c(c3)c3cc(-n5c(-c6ccccc6)nc6ccccc65)ccc3n4-c3ccccc3)nc(-c3ccc4c(c3)c3cc(-n5c(-c6ccccc6)nc6ccccc65)ccc3n4-c3ccccc3)n2)cc1 ; c1ccc(-c2nc(-c3ccc4c(c3)c3cc(N5c6ccccc6SC5c5ccccc5)ccc3n4-c3ccccc3)nc(-c3ccc4c(c3)c3cc(N5c6ccccc6SC5c5ccccc5)ccc3n4-c3ccccc3)n2)cc1 ; c1ccc(-c2nc(-c3ccc4c(c3)c3cc(N5c6ccccc6OC5c5ccccc5)ccc3n4-c3ccccc3)nc(-c3ccc4c(c3)c3cc(N5c6ccccc6OC5c5ccccc5)ccc3n4-c3ccccc3)n2)cc1]

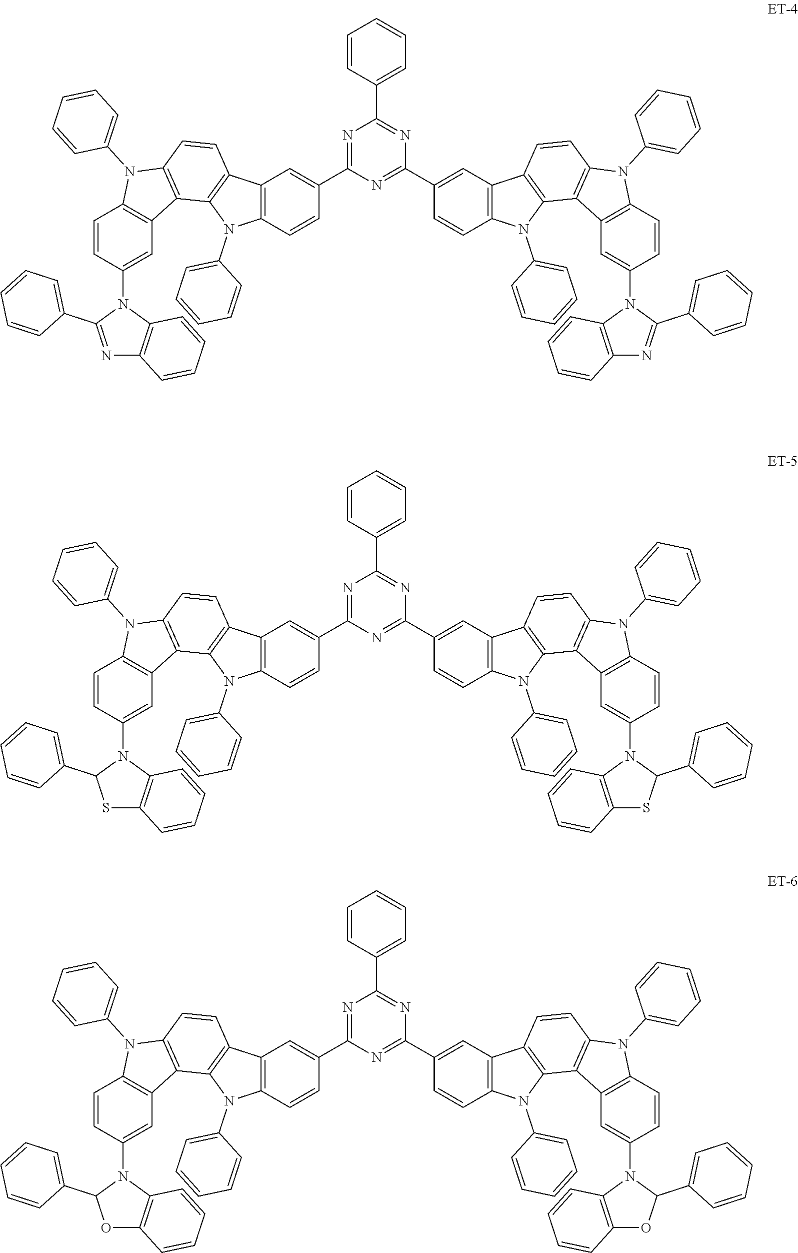

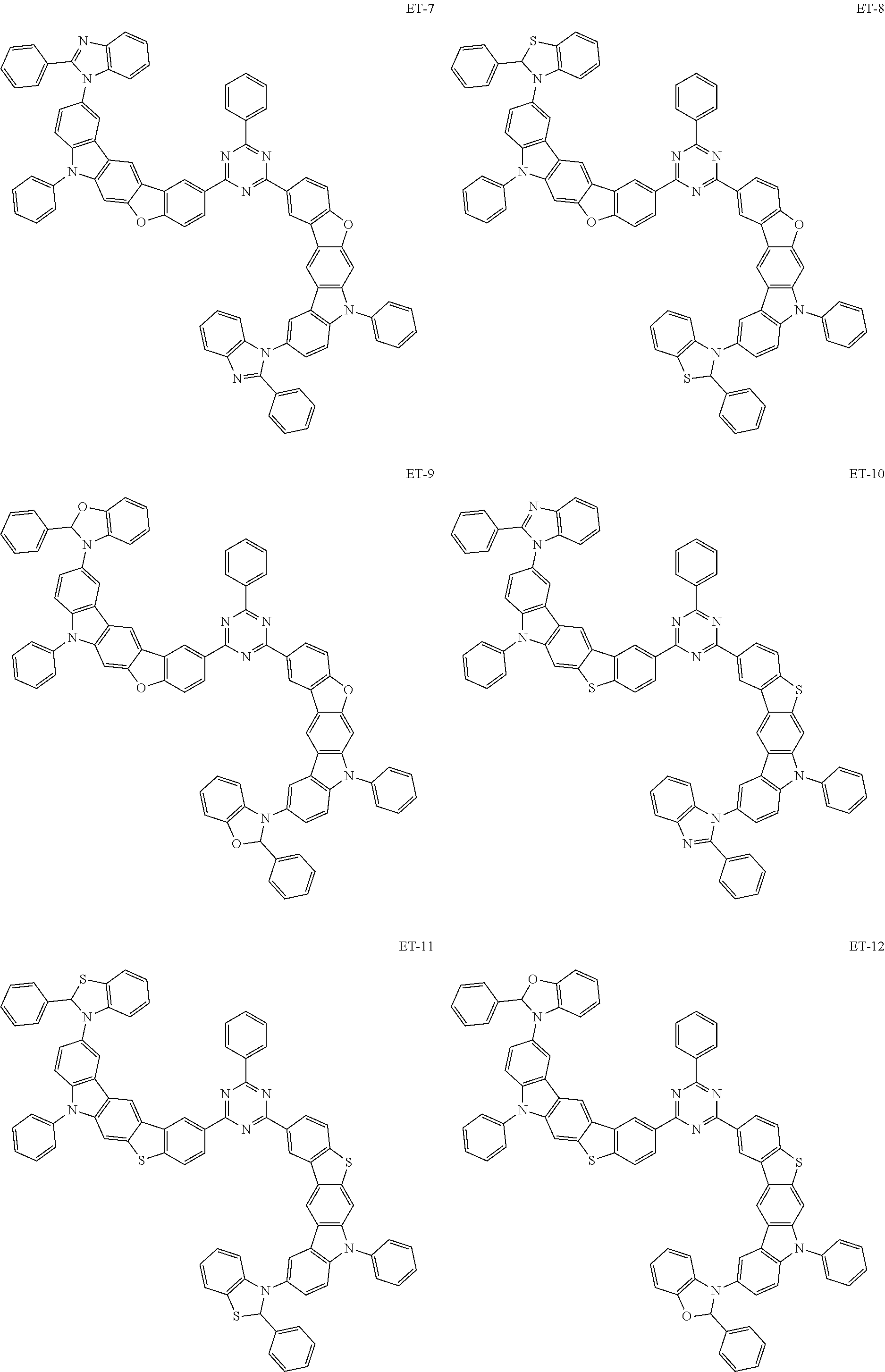

In the light emitting element ED of one or more embodiments shown in FIGS. 5 to 8, the emission layer EML may include a host and a dopant, and the emission layer EML may include a nitrogen-containing compound represented by Formula 1 as a host material. The emission layer EML may include the nitrogen-containing compound represented by Formula 1 as an electron transporting host, but the embodiment is not limited thereto. The nitrogen-containing compound of one or more embodiments may be a thermally activated delayed fluorescence host, a phosphorescent host, or a fluorescent host. The emission layer EML including the nitrogen-containing compound of one or more embodiments may be to emit thermally activated delayed fluorescence. The embodiment of the disclosure is not limited thereto, and the emission layer EML may be to emit phosphorescence or fluorescence.

The nitrogen-containing compound of one or more embodiments may include a core structure of a six-membered ring containing at least one nitrogen atom as a ring-forming atom, and may include a structure in which two heterocycle substituents selected from among Formula S-1 and Formula S-2 are connected to the core through a linker of a heteroarylene group having 6 or more ring-forming carbon atoms. Accordingly, the nitrogen-containing compound of one or more embodiments included in the emission layer EML may increase electrons in the emission layer EML and exhibit excellent or suitable electron transport properties. The nitrogen-containing compound of one or more embodiments has a structure with excellent or suitable electron transport properties, and thus, the light emitting element ED including the nitrogen-containing compound of one or more embodiments in the emission layer EML may efficiently utilize charges within the emission layer EML. Therefore, the light emitting element ED of one or more embodiments may exhibit both (e.g., simultaneously) high efficiency and long life characteristics, including the nitrogen-containing compound of one or more embodiments as an electron transporting host in the emission layer EML.

In one or more embodiments, the emission layer EML of the light emitting element ED may be to emit blue light. For example, the emission layer EML of the light emitting element ED of one or more embodiments may be to emit blue light in a wavelength range of about 440 nm to about 500 nm. However, the embodiment is not limited thereto, and the emission layer EML may be to emit green light or red light.

The nitrogen-containing compound of one or more embodiments may be included in the emission layer EML as a host material. For example, in the light emitting element ED of one or more embodiments, the emission layer EML may include at least one of the nitrogen-containing compounds shown in Compound Group 1 described herein as an electron transporting host. However, the use of the nitrogen-containing compound of one or more embodiments is not limited thereto.

In one or more embodiments, the emission layer EML may include a plurality of compounds. The emission layer EML of one or more embodiments may include two different hosts and dopants. The emission layer EML of one or more embodiments may include an electron transporting host, a hole transporting host, and a dopant that emits delayed fluorescence. The dopant emitting delayed fluorescence may be a thermally activated delayed fluorescence dopant. In some embodiments, the emission layer EML of one or more embodiments may further include a phosphorescent sensitizer. The embodiment of the disclosure is not limited thereto, and the emission layer EML of one or more embodiments may include an electron transporting host, a hole transporting host, and a phosphorescent dopant, or may include an electron transporting host, a hole transporting host, and a fluorescent dopant.

In one or more embodiments, the emission layer EML may include a second compound represented by Formula HT. In one or more embodiments, the second compound may be used as a hole transporting host material of the emission layer EML.

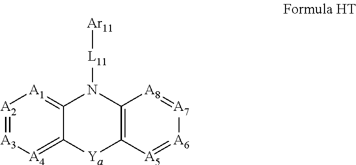

In Formula HT, A₁ to A₈ may each independently be N or CR₅₁. For example, all of A₁ to A₈ may be CR₅₁. In one or more embodiments, any one or at least one among (e.g., any one or at least one selected from among) A₁ to A₈ may (each) be N, and the rest may (each) be CR₅₁.

In Formula HT, L₁₁ may be a direct linkage, a substituted or unsubstituted arylene group having 6 to 30 ring-forming carbon atoms, or a substituted or unsubstituted heteroarylene group having 2 to 30 ring-forming carbon atoms. For example, L₁₁ may be a direct linkage, a substituted or unsubstituted phenylene group, a substituted or unsubstituted divalent biphenyl group, a substituted or unsubstituted divalent carbazole group, and/or the like, but the embodiment of the disclosure is not limited thereto.

In Formula HT, Ya may be a direct linkage, CR₅₂R₅₃, or SiR₅₄R₅₅. For example, it may refer to that the two benzene rings linked to the nitrogen atom in Formula HT are linked via a direct linkage,

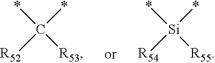

In Formula HT, if (e.g., when) Ya is a direct linkage, the second compound represented by Formula HT may include a carbazole moiety.

In Formula HT, Ar₁₁ may be a substituted or unsubstituted aryl group having 6 to 30 ring-forming carbon atoms, or a substituted or unsubstituted heteroaryl group having 2 to 30 ring-forming carbon atoms. For example, Ar₁₁ may be a substituted or unsubstituted carbazole group, a substituted or unsubstituted dibenzofuran group, a substituted or unsubstituted dibenzothiophene group, a substituted or unsubstituted biphenyl group, and/or the like, but the embodiment of the disclosure is not limited thereto.

In Formula HT, R₅₁ to R₅₅ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 60 ring-forming carbon atoms, or a substituted or unsubstituted heteroaryl group having 2 to 60 ring-forming carbon atoms. In one or more embodiments, each of R₅₁ to R₅₅ may be bonded to an adjacent group to form a ring. For example, R₅₁ to R₅₅ may each independently be a hydrogen atom or a deuterium atom. R₅₁ to R₅₅ may each independently be an unsubstituted methyl group or an unsubstituted phenyl group.

In one or more embodiments, the second compound represented by Formula HT may be represented by any one among the compounds represented by Compound Group 2. The emission layer EML may include at least one among (e.g., at least one selected from among) the compounds represented by Compound Group 2 as a hole transporting host material.

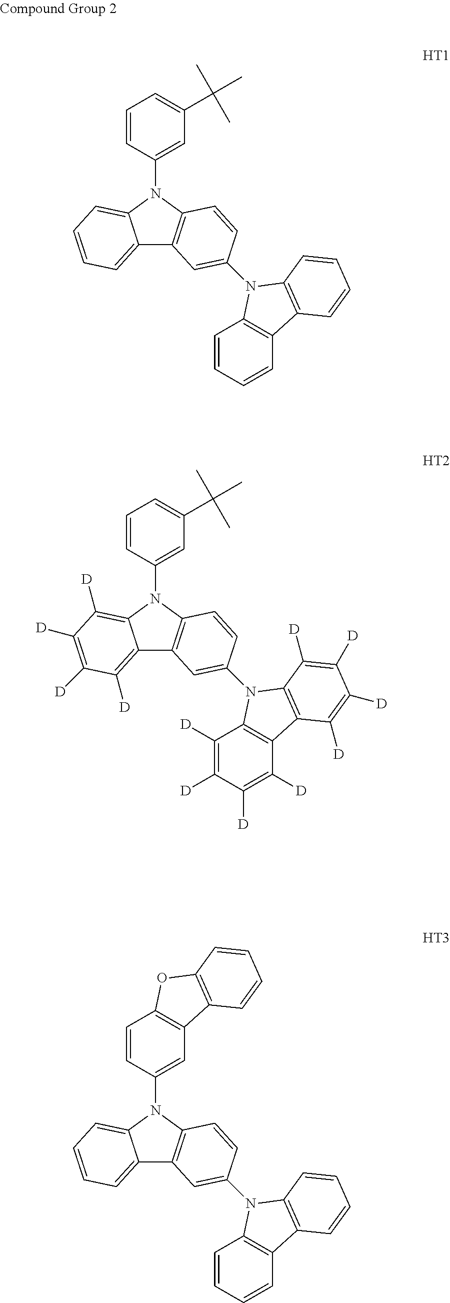 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

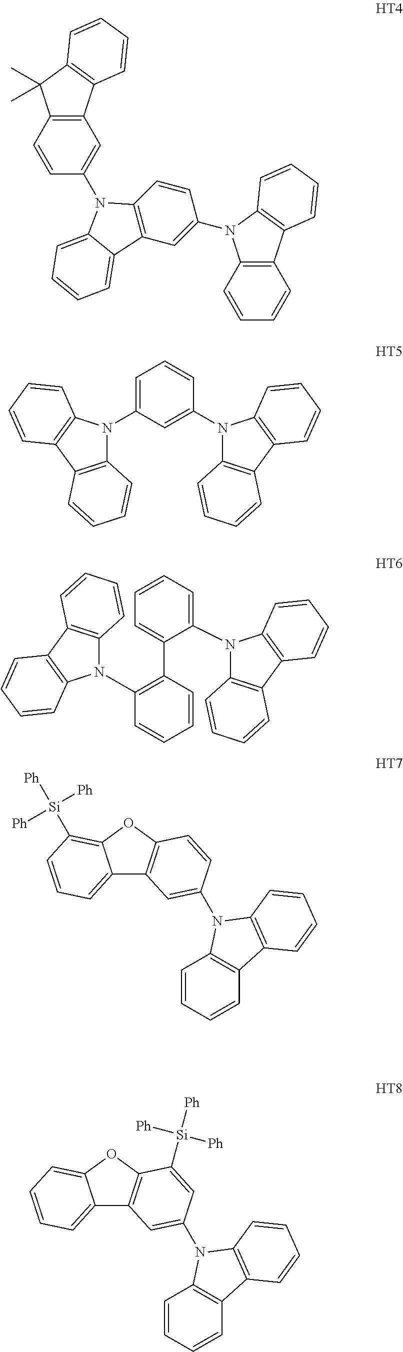 [SMILES: CC1(C)c2ccccc2-c2cc(-n3c4ccccc4c4cc(-n5c6ccccc6c6ccccc65)ccc43)ccc21 ; c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1 ; c1ccc([Si](c2ccccc2)(c2ccccc2)c2cc(-n3c4ccccc4c4ccccc43)cc3c2oc2ccccc23)cc1]

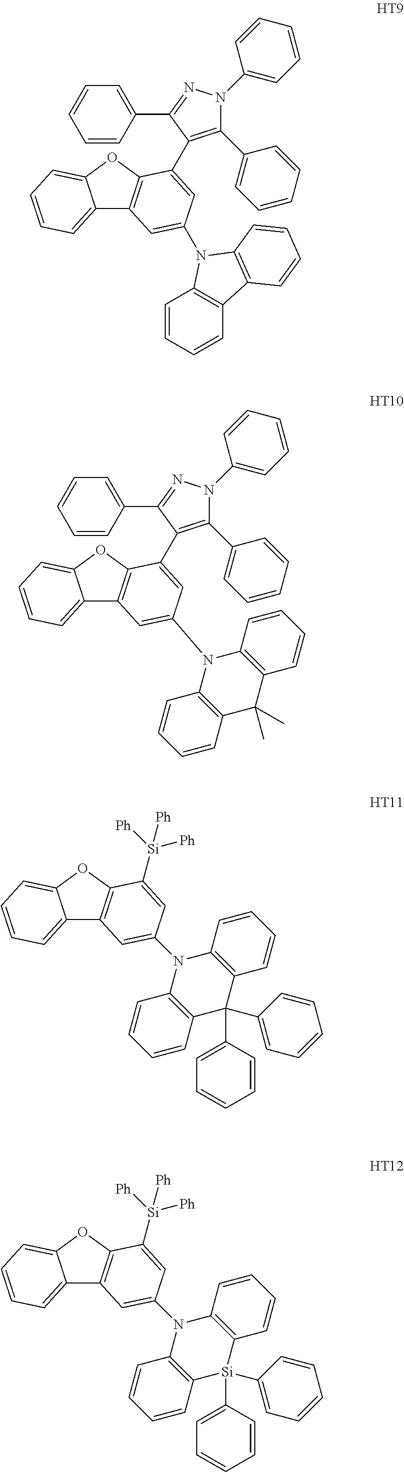 [SMILES: c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1 ; c1ccc([Si]2(c3ccccc3)c3ccccc3N(c3cc([Si](c4ccccc4)(c4ccccc4)c4ccccc4)c4oc5ccccc5c4c3)c3ccccc32)cc1]

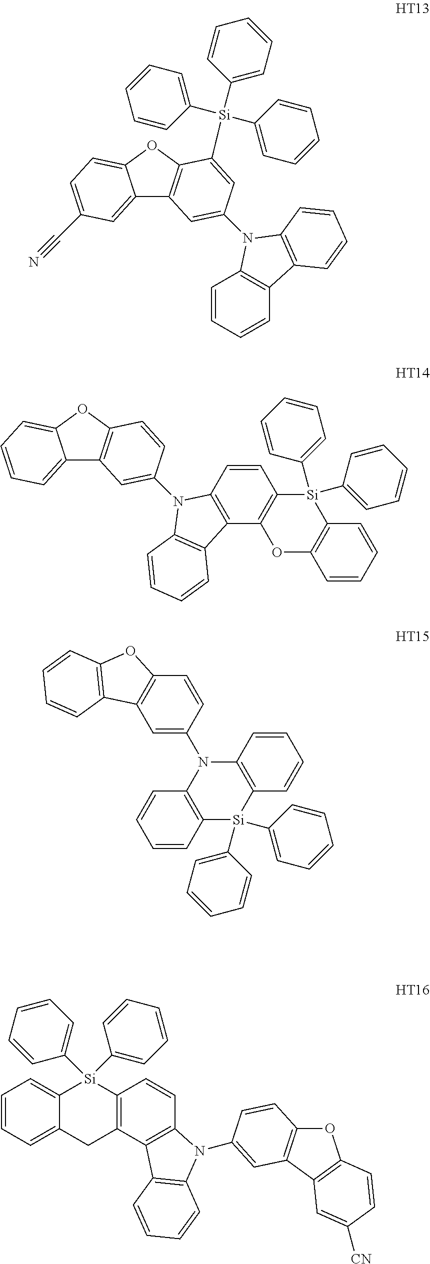 [SMILES: N#Cc1ccc2oc3c([Si](c4ccccc4)(c4ccccc4)c4ccccc4)cc(-n4c5ccccc5c5ccccc54)cc3c2c1 ; c1ccc([Si]2(c3ccccc3)c3ccccc3Oc3c2ccc2c3c3ccccc3n2-c2ccc3oc4ccccc4c3c2)cc1 ; c1ccc([Si]2(c3ccccc3)c3ccccc3N(c3ccc4oc5ccccc5c4c3)c3ccccc32)cc1 ; N#Cc1ccc2oc3ccc(-n4c5ccccc5c5c6c(ccc54)[Si](c4ccccc4)(c4ccccc4)c4ccccc4C6)cc3c2c1]

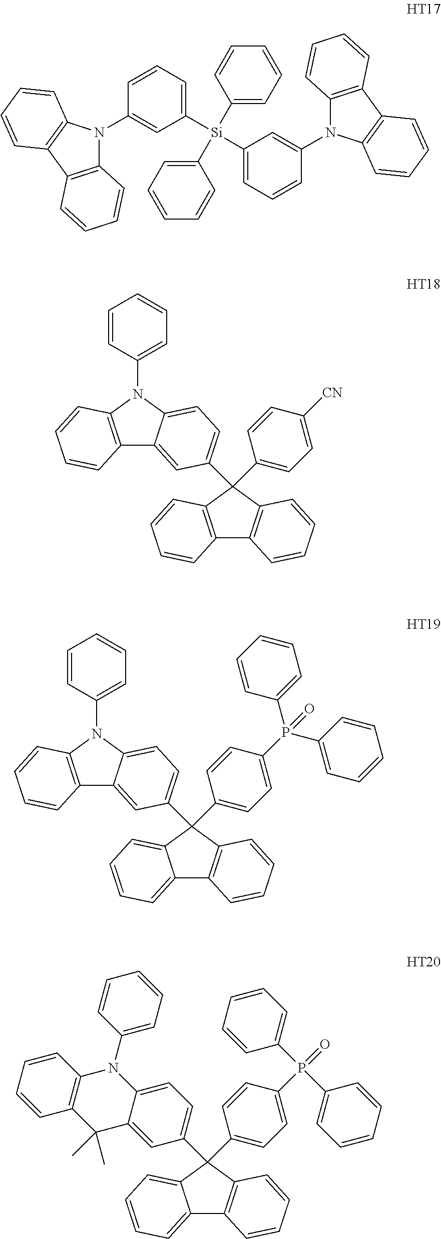 [SMILES: c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1 ; O=P(c1ccccc1)(c1ccccc1)c1ccc(C2(c3ccc4c(c3)c3ccccc3n4-c3ccccc3)c3ccccc3-c3ccccc32)cc1 ; CC1(C)c2ccccc2N(c2ccccc2)c2ccc(C3(c4ccc(P(=O)(c5ccccc5)c5ccccc5)cc4)c4ccccc4-c4ccccc43)cc21]

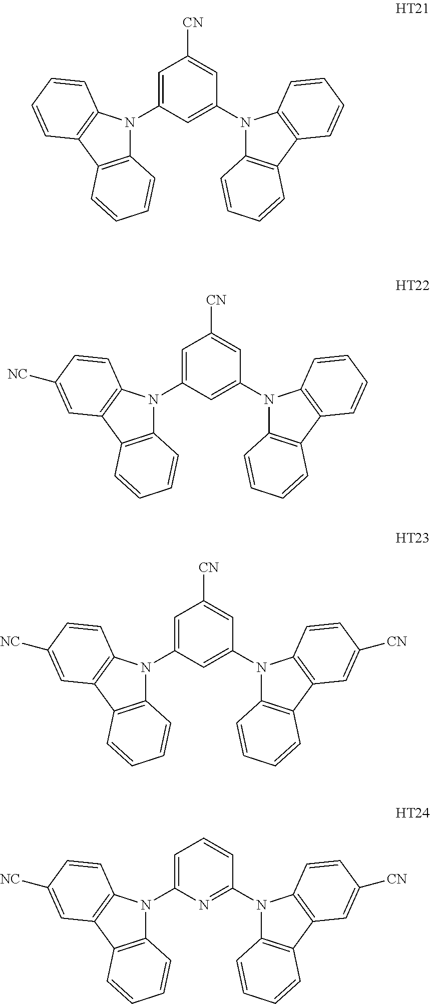 [SMILES: N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1 ; N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)n1]

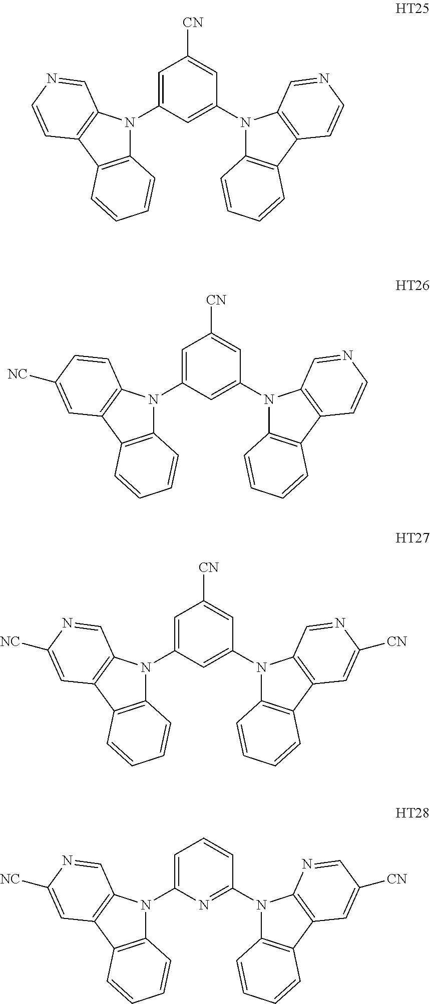 [SMILES: N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1 ; N#Cc1cc(-n2c3ccccc3c3cc(C#N)ncc32)cc(-n2c3ccccc3c3cc(C#N)ncc32)c1 ; N#Cc1cnc2c(c1)c1ccccc1n2-c1cccc(-n2c3ccccc3c3cc(C#N)ncc32)n1]

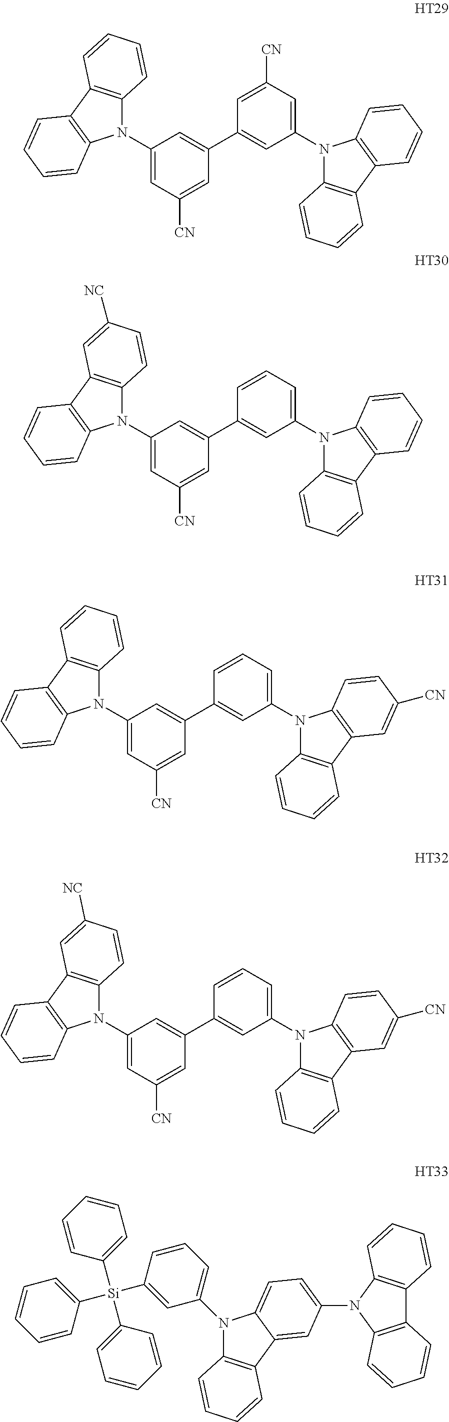 [SMILES: N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1 ; N#Cc1cc(-c2cccc(-n3c4ccccc4c4cc(C#N)ccc43)c2)cc(-n2c3ccccc3c3cc(C#N)ccc32)c1 ; c1ccc([Si](c2ccccc2)(c2ccccc2)c2cccc(-n3c4ccccc4c4cc(-n5c6ccccc6c6ccccc65)ccc43)c2)cc1]

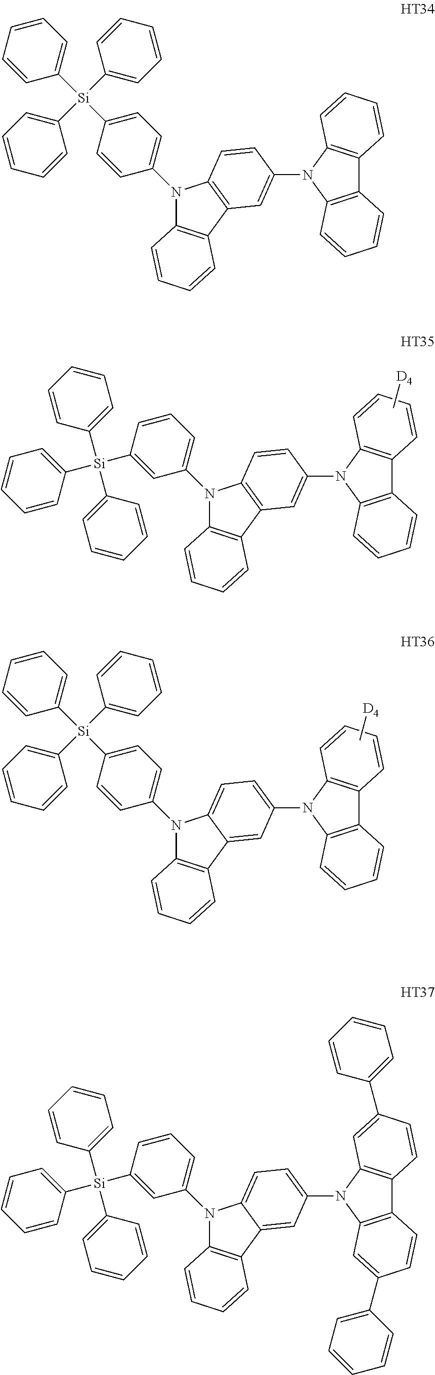

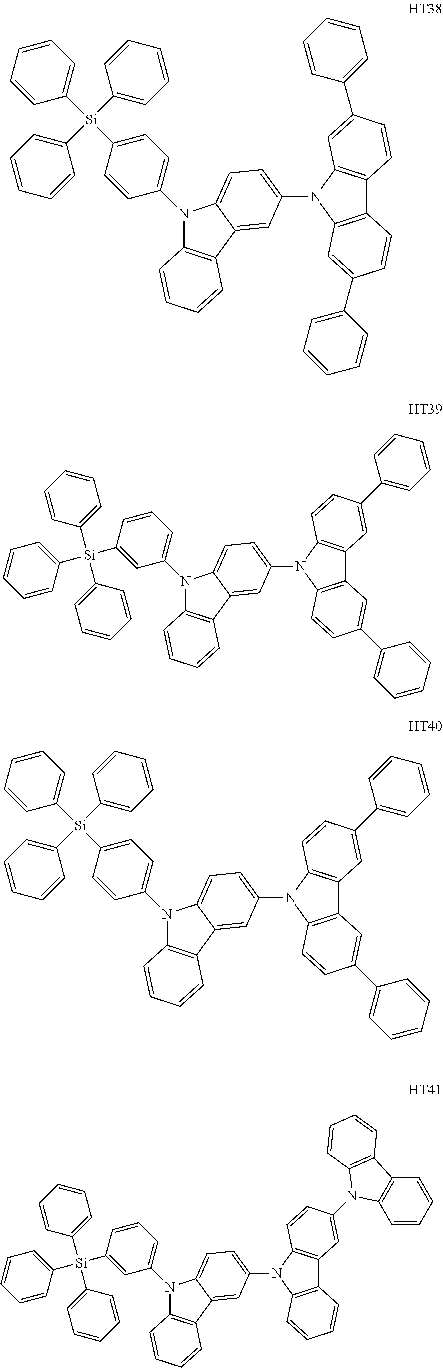 [SMILES: c1ccc(-c2ccc3c4ccc(-c5ccccc5)cc4n(-c4ccc5c(c4)c4ccccc4n5-c4ccc([Si](c5ccccc5)(c5ccccc5)c5ccccc5)cc4)c3c2)cc1 ; c1ccc(-c2ccc3c(c2)c2cc(-c4ccccc4)ccc2n3-c2ccc3c(c2)c2ccccc2n3-c2cccc([Si](c3ccccc3)(c3ccccc3)c3ccccc3)c2)cc1 ; c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1 ; c1ccc([Si](c2ccccc2)(c2ccccc2)c2cccc(-n3c4ccccc4c4cc(-n5c6ccccc6c6cc(-n7c8ccccc8c8ccccc87)ccc65)ccc43)c2)cc1]

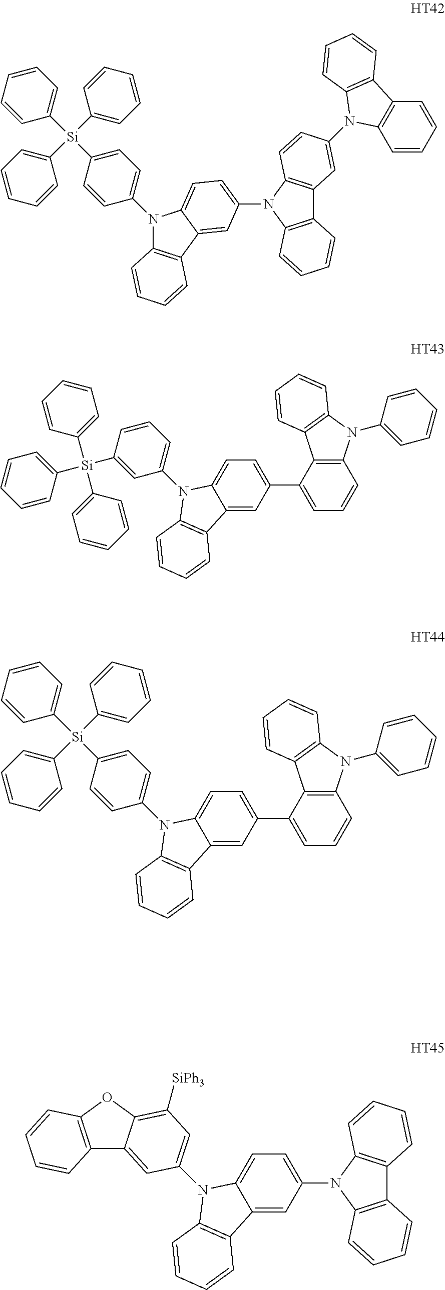

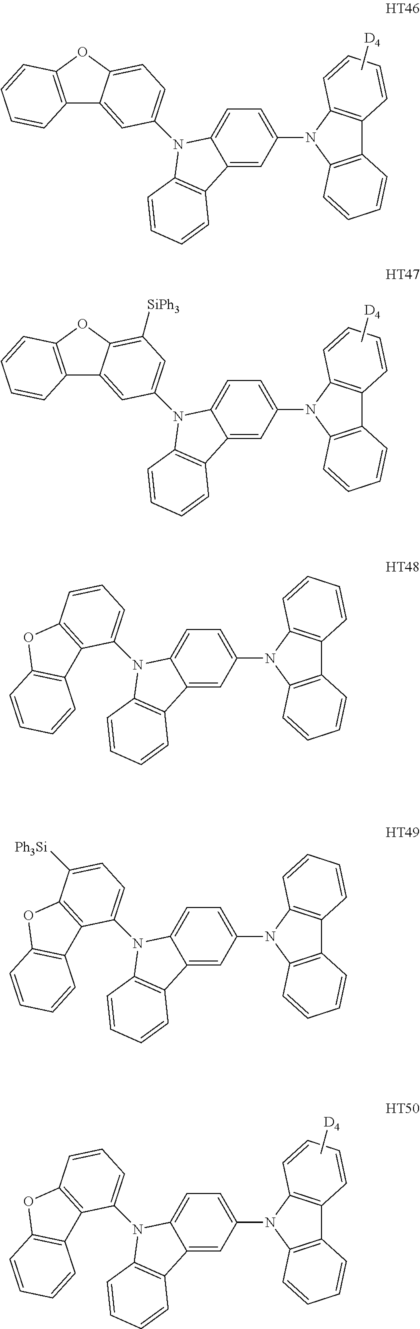

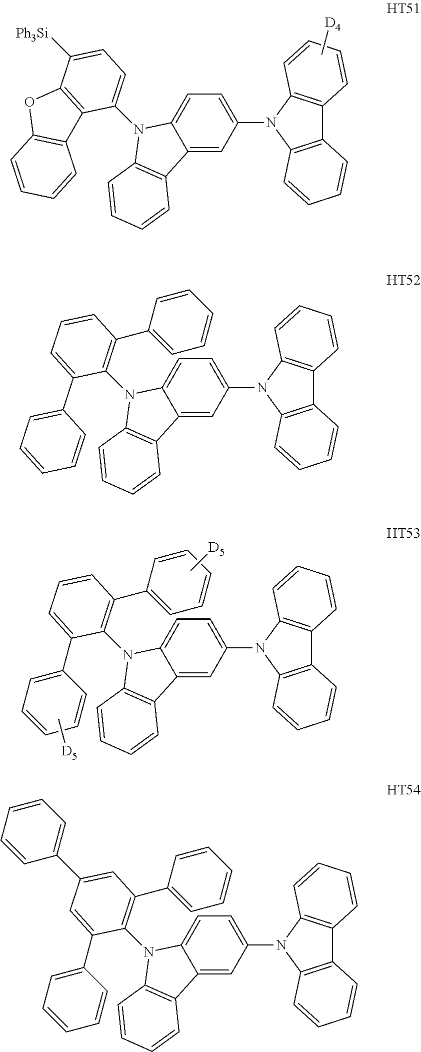

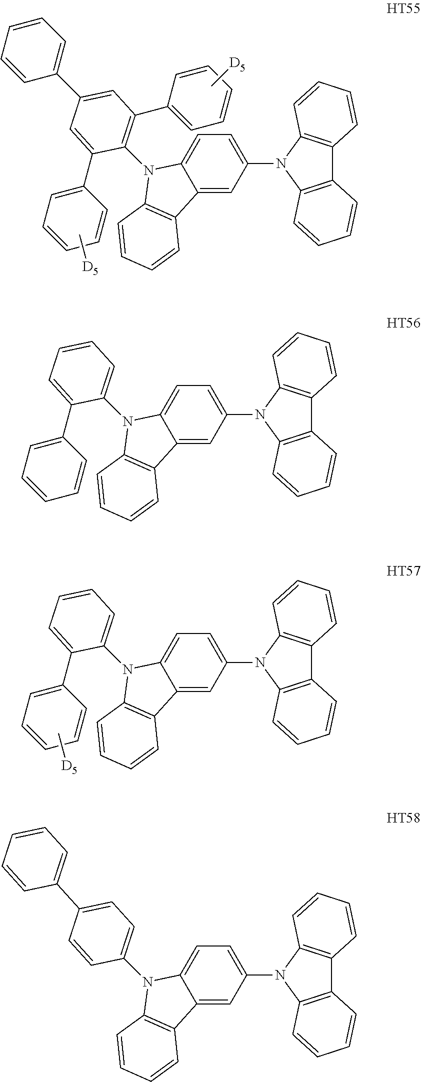

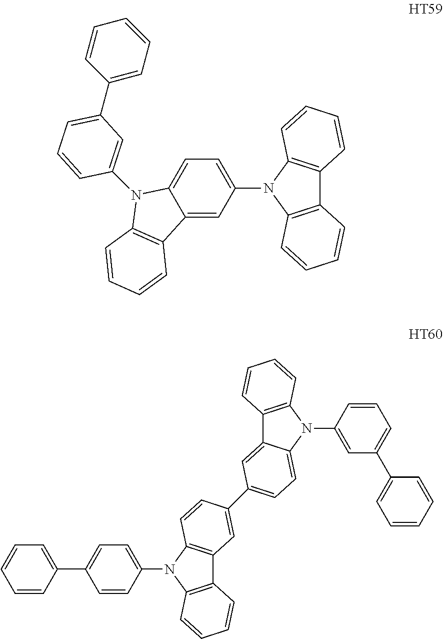 [SMILES: c1ccc(-c2cccc(-n3c4ccccc4c4cc(-n5c6ccccc6c6ccccc65)ccc43)c2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

In embodiment compounds presented in Compound Group 2, “D” may refer to a deuterium atom, and “Ph” may refer to a substituted or unsubstituted phenyl group. For example, in embodiment compounds presented in Compound Group 2, “Ph” may refer to an unsubstituted phenyl group.

In one or more embodiments, the emission layer EML may include a third compound represented by Formula TA. The third compound may be included as a dopant in the emission layer EML. The third compound may be used as a thermally activated delayed fluorescence dopant material of the emission layer EML.

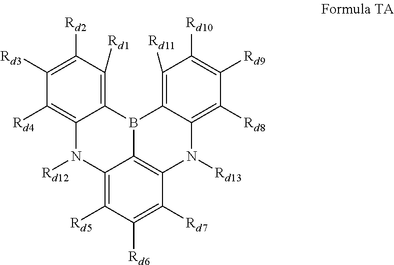

R_{d1} to R_{d13} may each independently be a hydrogen atom, a deuterium atom, a halogen atom, a cyano group, a substituted or unsubstituted amine group, a substituted or unsubstituted boron group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, a substituted or unsubstituted heteroaryl group having 2 to 30 ring-forming carbon atoms, or bonded to an adjacent group to form a ring.

For example, a compound represented by Formula TA may include compound TA-1 shown in Compound Group 3. The emission layer EML may include compound TA-1 shown in Compound Group 3 as a thermally activated delayed fluorescence dopant material. However, the thermally activated delayed fluorescence dopant is not limited to the compounds of Compound Group 3.

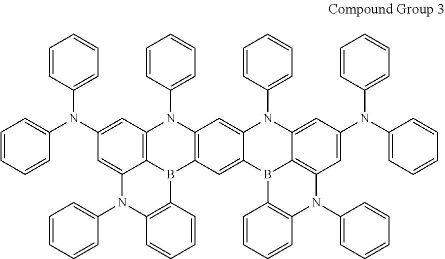 [SMILES: c1ccc(N(c2ccccc2)c2cc3c4c(c2)N(c2ccccc2)c2cc5c(cc2B4c2ccccc2N3c2ccccc2)B2c3ccccc3N(c3ccccc3)c3cc(N(c4ccccc4)c4ccccc4)cc(c32)N5c2ccccc2)cc1]

In one or more embodiments, the emission layer EML may include a fourth compound represented by Formula D-1. For example, the emission layer EML may include an organometallic complex containing platinum (Pt) as a central metal atom and ligands bonded to the central metal atom, as a sensitizer or a dopant. In the light emitting element ED of one or more embodiments, the emission layer EML may include the first compound, the second compound, and the third compound described herein, and may further include the fourth compound represented by Formula D-1 as a phosphorescent sensitizer. The embodiment of the disclosure is not limited thereto, and in the light emitting element ED of one or more embodiments, the emission layer EML may include the fourth compound represented by Formula D-1 as a phosphorescent dopant.

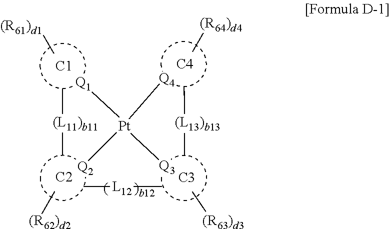

In Formula D-1, Q₁ to Q₄ may each independently be C or N. In Formula D-1, C1 to C4 may each independently be a substituted or unsubstituted hydrocarbon ring having 5 to 30 ring-forming carbon atoms, or a substituted or unsubstituted heterocycle having 2 to 30 ring-forming carbon atoms.

In Formula D-1, L₁₁ to L₁₃ may each independently be a direct linkage,

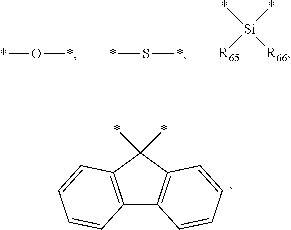

a substituted or unsubstituted alkylene group having 1 to 20 carbon atoms, a substituted or unsubstituted arylene group having 6 to 30 ring-forming carbon atoms, or a substituted or unsubstituted heteroarylene group having 2 to 30 ring-forming carbon atoms. In L₁₁ to L₁₃, “ [symbol] ” refers to a part linked to C1 to C4.

In Formula D-1, b11 to b13 may each independently be 0 or 1. If b11 is 0, C1 and C2 may not be linked to each other. If b12 is 0, C2 and C3 may not be linked to each other. If b13 is 0, C3 and C4 may not be linked to each other.

In Formula D-1, R₆₁ to R₆₆ may each independently be a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 60 ring-forming carbon atoms, or a substituted or unsubstituted heteroaryl group having 2 to 60 ring-forming carbon atoms. In one or more embodiments, each of R₆₁ to R₆₆ may be bonded to an adjacent group to form a ring. R₆₁ to R₆₆ may each independently be a substituted or unsubstituted methyl group, or a substituted or unsubstituted t-butyl group.

In Formula D-1, d1 to d4 may each independently be an integer of 0 to 4. In Formula D-1, if each of d1 to d4 is 0, the fourth compound may not be substituted with each of R₆₁ to R₆₄. The case where each of d1 to d4 is 4 and R₆₁'s to R₆₄' are each hydrogen atoms may be the same as the case where each of d1 to d4 is 0. When each of d1 to d4 is an integer of 2 or more, a plurality of R₆₁'s to R₆₄'s may each be the same or at least one among the plurality of R₆₁'s to R₆₄'s may be different from the others.

In Formula D-1, C1 to C4 may each independently be a substituted or unsubstituted hydrocarbon ring or a substituted or unsubstituted heterocycle represented by any one among (e.g., any one selected from among) C-1 to C-5:

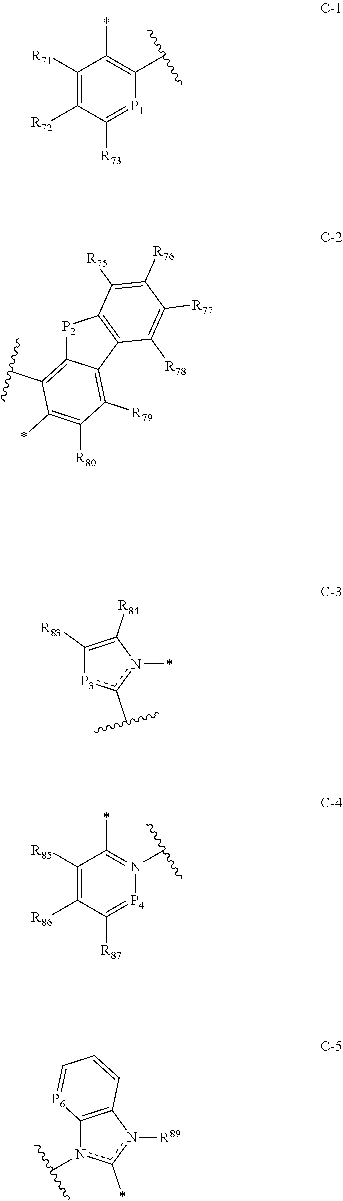

In C-1 to C-5, P₁ may be

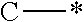 [SMILES: *C]

or CR₇₄, P₂ may be

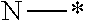 [SMILES: *N]

or NR₈₁, P₃ may be

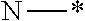 [SMILES: *N]

or NR₈₂, P₄ may be

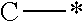 [SMILES: *C]

or CR₈₈, and P₅ may be

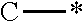 [SMILES: *C]

or CR₉₀. R₇₁ to R₉₀ may each independently be a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring.

In some embodiments, in C-1 to C-5,

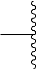

corresponds to a part linked to Pt that is a central metal atom, and “ [symbol] ” corresponds to a part linked to a neighboring cyclic group (C1 to C4) or a linker (L₁₁ to L₁₃).

The emission layer EML may include the first compound, the second compound, the third compound, and the fourth compound. In the emission layer EML, the first compound and the second compound may form an exciplex, and the energy may be transferred from the exciplex to the fourth compound and the third compound, thereby emitting light. In one or more embodiments, the fourth compound may be a sensitizer. The fourth compound included in the emission layer EML in the light emitting element ED of one or more embodiments may serve as a sensitizer to deliver energy from the host to the third compound that is a light emitting dopant. For example, the fourth compound serving as an auxiliary dopant accelerates energy delivery to the third compound that is a light emitting dopant, thereby increasing the emission ratio of the third compound. Therefore, the emission layer EML of one or more embodiments may improve luminous efficiency. In some embodiments, if (e.g., when) the energy delivery to the third compound is increased, an exciton formed in the emission layer EML is not accumulated inside the emission layer EML and emits light rapidly, and thus deterioration of the device may be reduced. Therefore, the service life of the light emitting element ED of one or more embodiments may increase.

The embodiment of the disclosure is not limited thereto, and the emission layer EML of one or more embodiments may include the first compound, the second compound, and the fourth compound, and in this case, the fourth compound may be included as a light emitting dopant.

In one or more embodiments, the fourth compound represented by Formula D-1 may represented at least one among (e.g., at least one selected from among) the compounds represented by Compound Group 4. The emission layer EML may include at least one among (e.g., at least one selected from among) the compounds shown in Compound Group 4 as a phosphorescent sensitizer or a phosphorescent dopant material. However, the phosphorescent sensitizer or the phosphorescent dopant is not limited to the compounds of Compound Group 4.

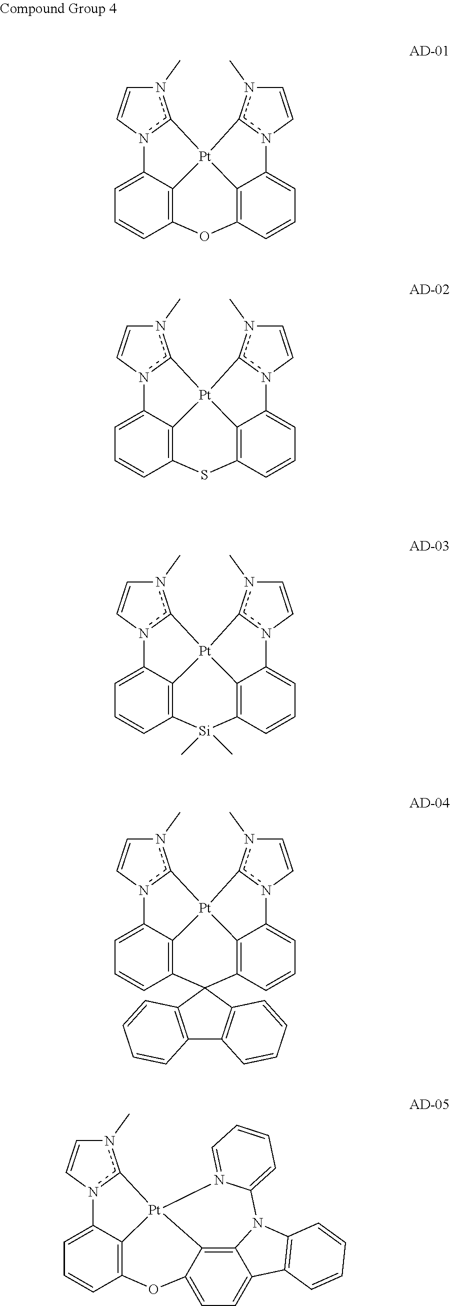

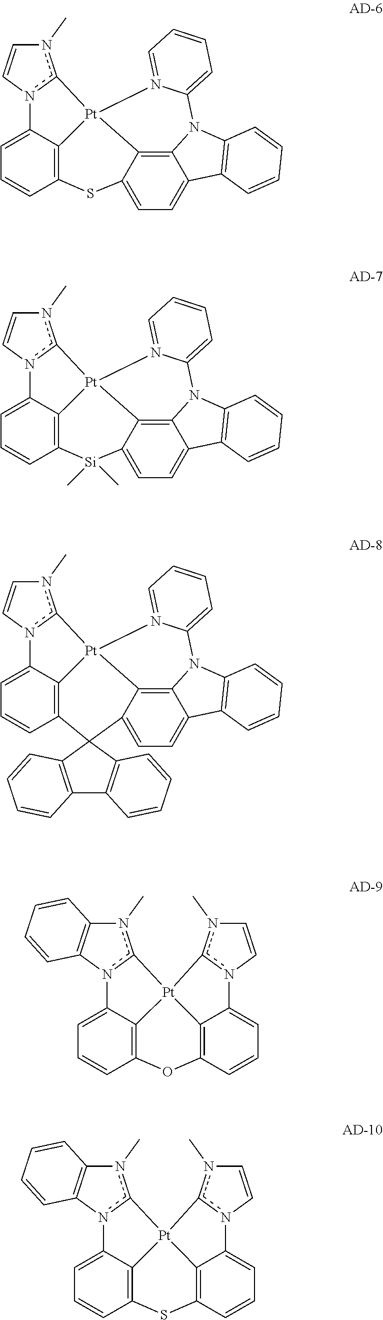

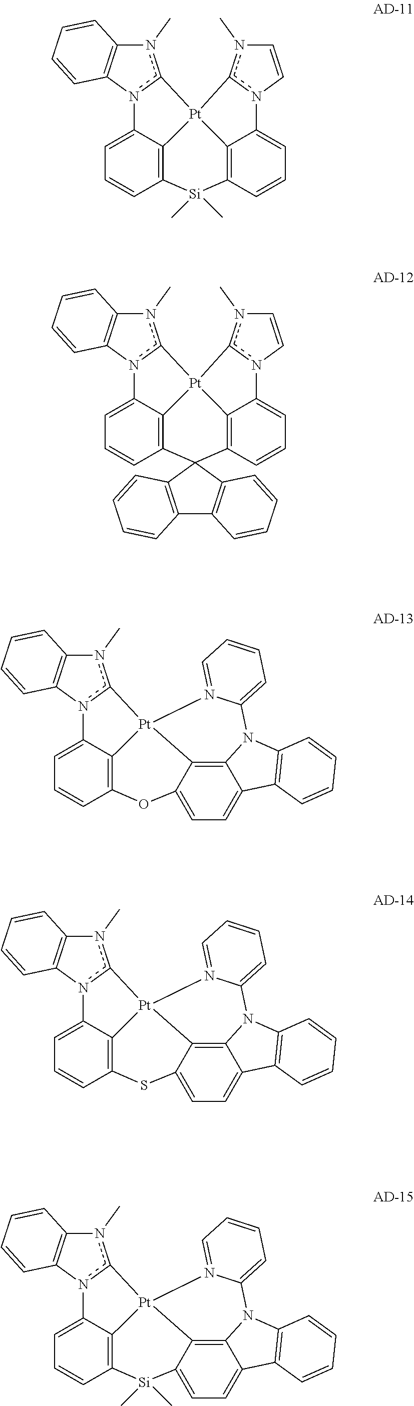

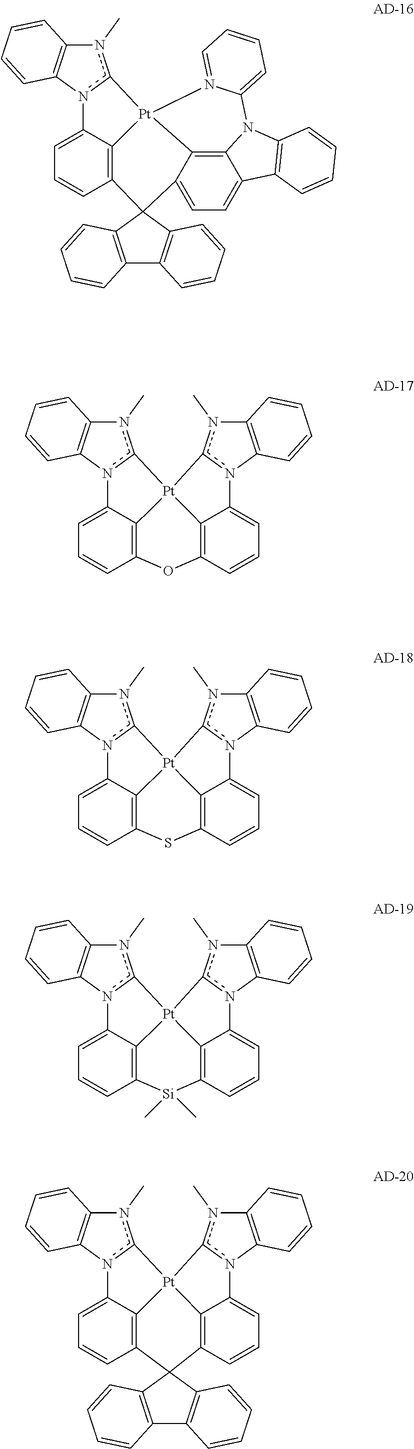

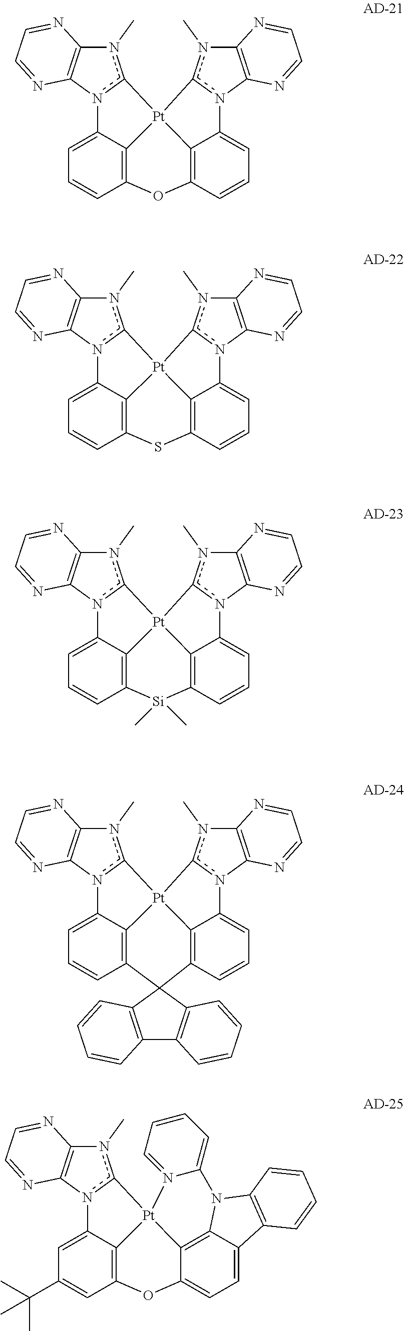

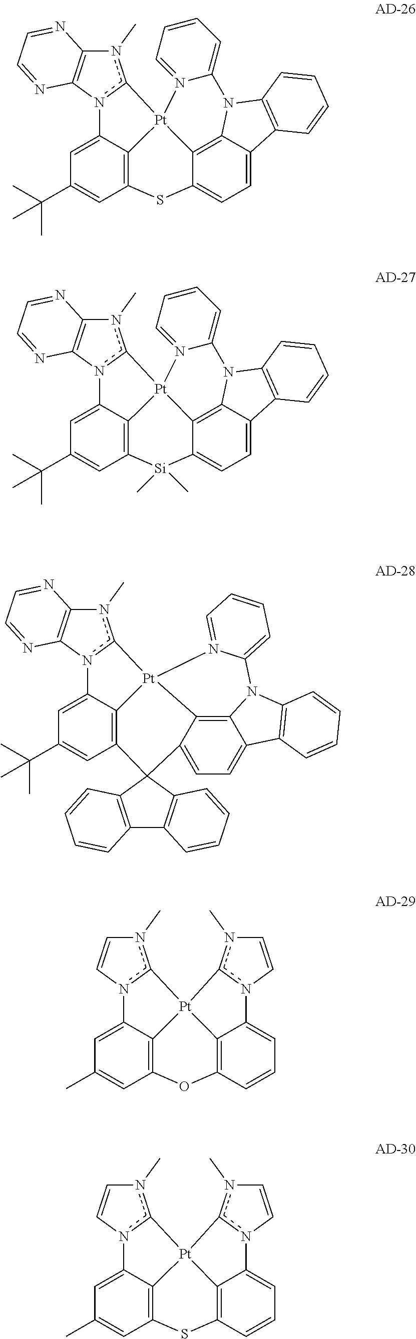

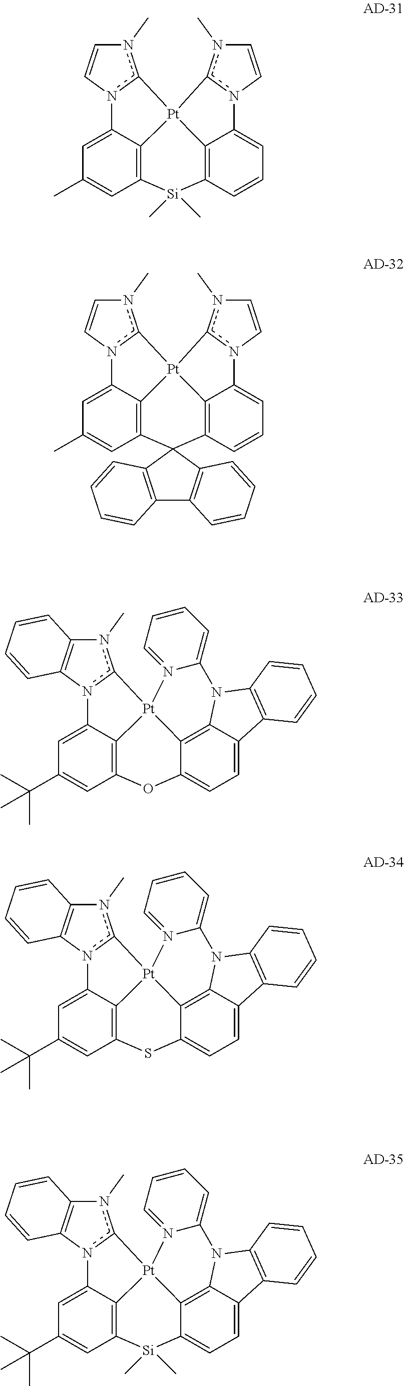

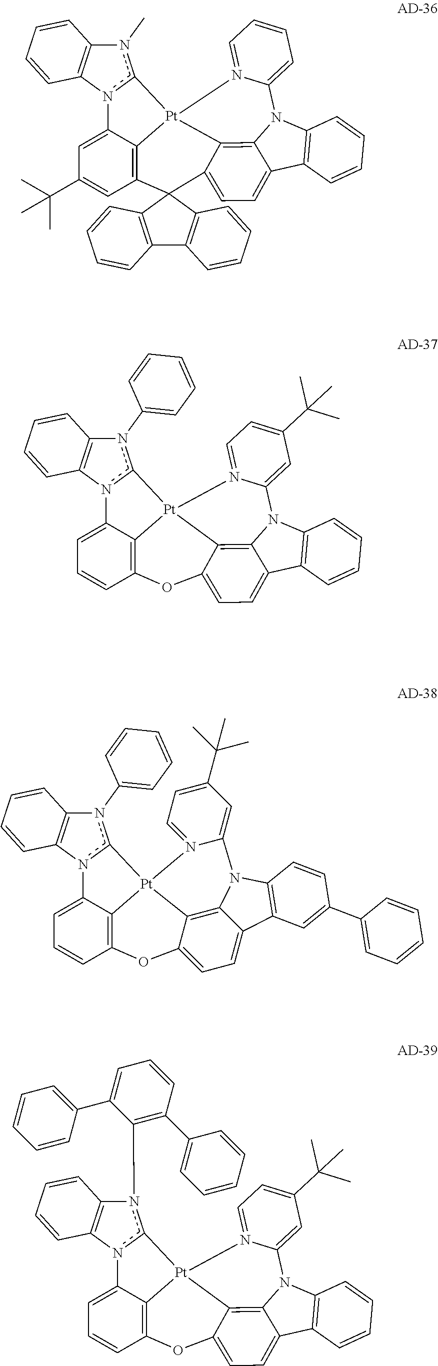

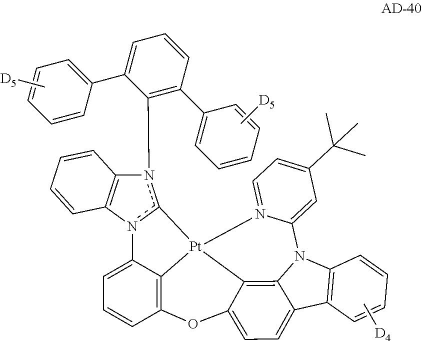

The emission layer EML of one or more embodiments may include the first compound which is a nitrogen-containing compound, and at least one of the second to fourth compounds. For example, the emission layer EML may include the first compound, the second compound, and the third compound.

In some embodiments, the emission layer EML may include all of the first compound, the second compound, the third compound, and the fourth compound. For example, the emission layer EML may include a combination of two host materials and two dopant materials. In the light emitting element ED of one or more embodiments, the emission layer EML include the first compound and the second compound that are two different hosts, the third compound emitting delayed fluorescence, and the fourth compound containing an organometallic complex, and may thus exhibit excellent or suitable luminous efficiency. The embodiment of the disclosure is not limited thereto, and the emission layer EML may include the first compound and the second compound as hosts, and the fourth compound as a dopant.

In the light emitting element ED of one or more embodiments, the emission layer EML may include an anthracene derivative, a pyrene derivative, a fluoranthene derivative, a chrysene derivative, a dihydrobenzanthracene derivative, or a triphenylene derivative. For example, the emission layer EML may include the anthracene derivative or the pyrene derivative.

In each light emitting element ED of embodiments illustrated in FIGS. 5 to 8, the emission layer EML may further include a suitable host and dopant besides the herein-described host and dopant, and for example the emission layer EML may include a compound represented by Formula E-1. The compound represented by Formula E-1 may be used as a fluorescent host material.

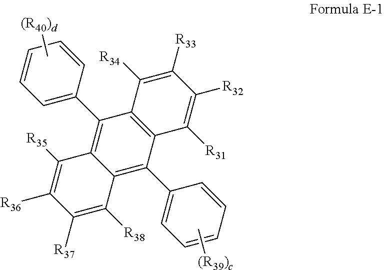

In Formula E-1, R₃₁ to R₄₀ may each independently be a hydrogen atom, a deuterium atom, a halogen atom, a substituted or unsubstituted silyl group, a substituted or unsubstituted thio group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In one or more embodiments, R₃₁ to R₄₀ may be bonded to an adjacent group to form a saturated hydrocarbon ring or an unsaturated hydrocarbon ring, a saturated heterocycle, or an unsaturated heterocycle.

In Formula E-1, c and d may each independently be an integer of 0 to 5. Formula E-1 may be represented by any one among (e.g., any one selected from among) Compound E1 to Compound E21:

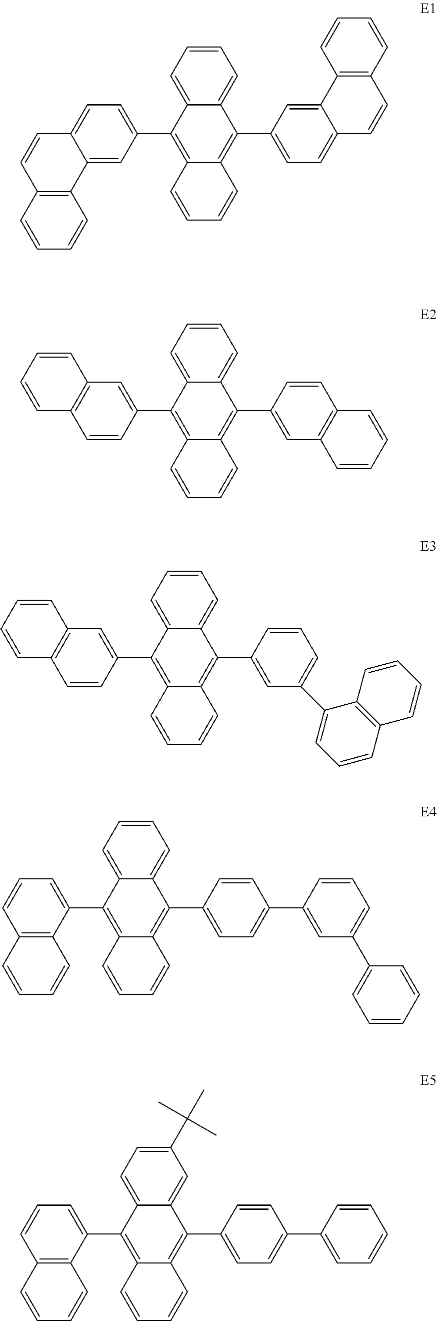

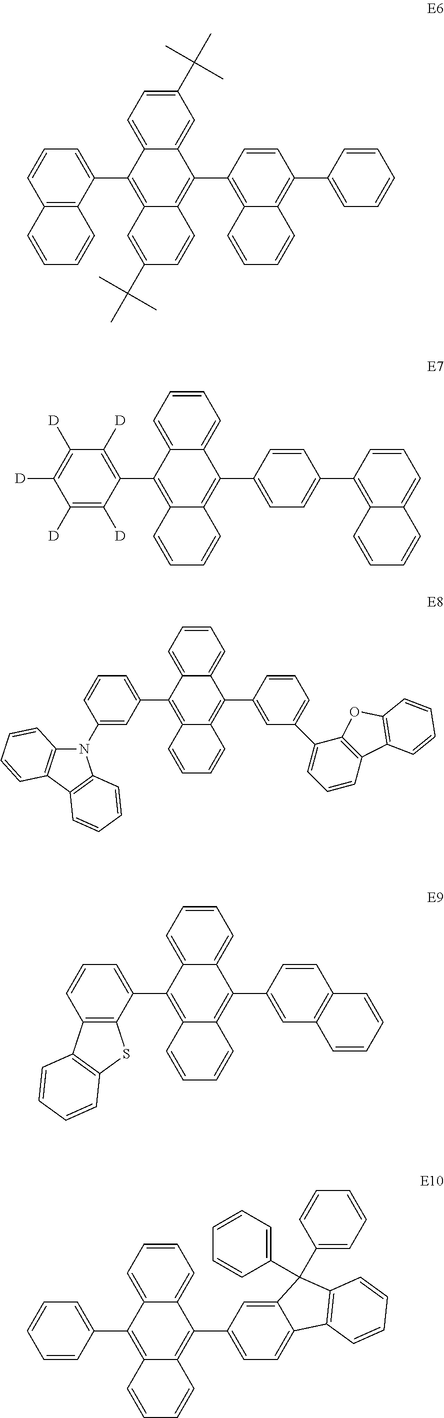

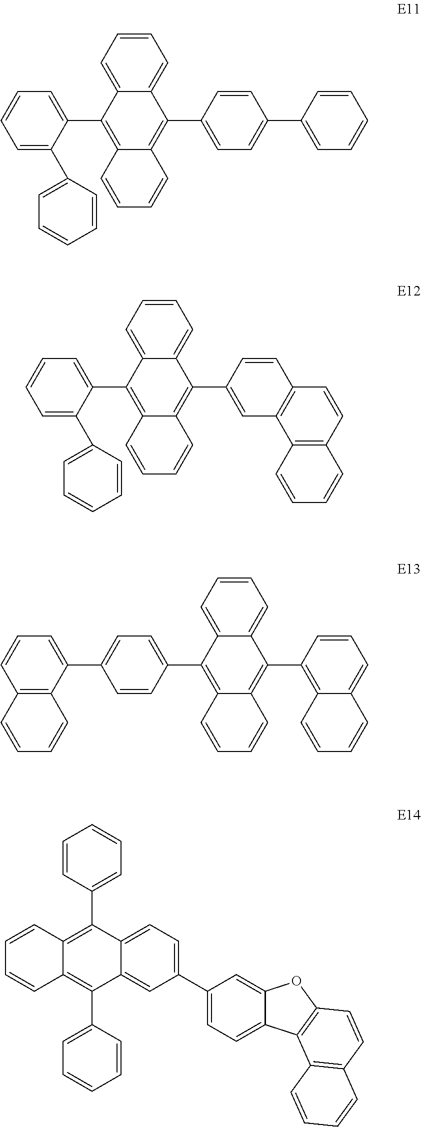

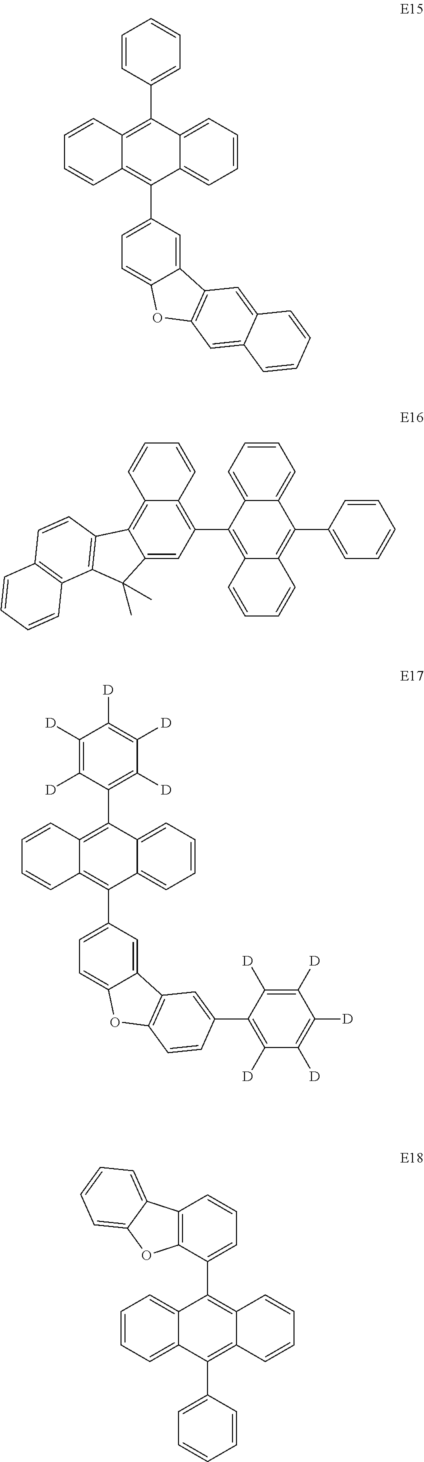

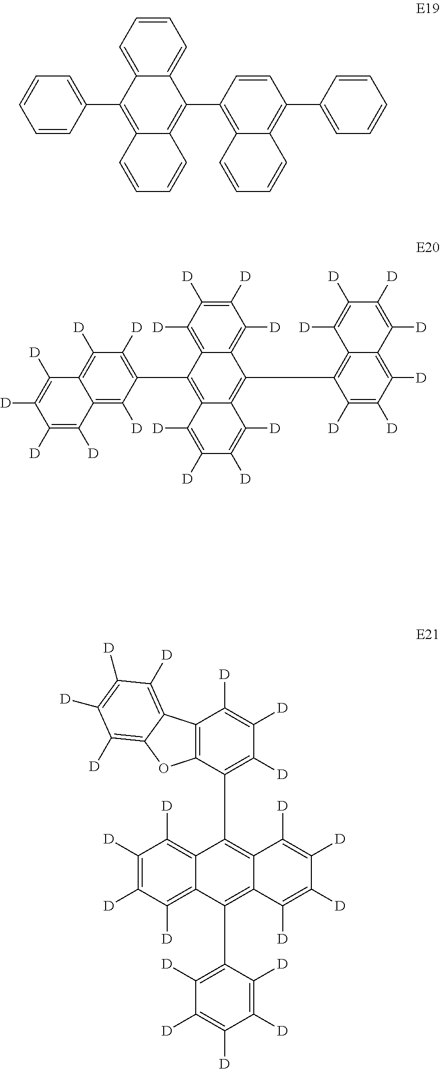

In one or more embodiments, the emission layer EML may include a compound represented by Formula E-2a or Formula E-2b. The compound represented by Formula E-2a or Formula E-2b may be used as a host material for phosphorescent light emitting element.

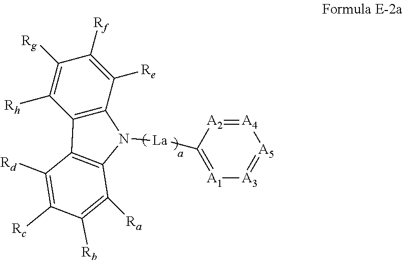

In Formula E-2a, a may be an integer of 0 to 10, and Lₐ may be a direct linkage, a substituted or unsubstituted arylene group having 6 to 30 ring-forming carbon atoms, or a substituted or unsubstituted heteroarylene group having 2 to 30 ring-forming carbon atoms. In one or more embodiments, if (e.g., when) a is an integer of 2 or greater, a plurality of Lₐ's may each independently be a substituted or unsubstituted arylene group having 6 to 30 ring-forming carbon atoms, or a substituted or unsubstituted heteroarylene group having 2 to 30 ring-forming carbon atoms.

In some embodiments, in Formula E-2a, A₁ to A₅ may each independently be N or CRᵢ. Rₐ to Rᵢ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. Rₐ to Rᵢ may be bonded to an adjacent group to form a hydrocarbon ring or a heterocycle containing N, O, S, and/or the like, as a ring-forming atom.

In one or more embodiments, in Formula E-2a, two or three selected from among A₁ to A₅ may be N, and the rest may be CRᵢ.

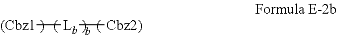

In Formula E-2b, Cbz1 and Cbz2 may each independently be an unsubstituted carbazole group, or a carbazole group substituted with an aryl group having 6 to 30 ring-forming carbon atoms. L_{b} is a direct linkage, a substituted or unsubstituted arylene group having 6 to 30 ring-forming carbon atoms, or a substituted or unsubstituted heteroarylene group having 2 to 30 ring-forming carbon atoms. In one or more embodiments, b is an integer of 0 to 10, and if (e.g., when) b is an integer of 2 or more, a plurality of L_{b}'s may each independently be a substituted or unsubstituted arylene group having 6 to 30 ring-forming carbon atoms, or a substituted or unsubstituted heteroarylene group having 2 to 30 ring-forming carbon atoms.

The compound represented by Formula E-2a or Formula E-2b may be represented by any one among the compounds of Compound Group E-2. However, the compounds listed in Compound Group E-2 are only examples, and the compound represented by Formula E-2a or Formula E-2b is not limited to those represented in Compound Group E-2.

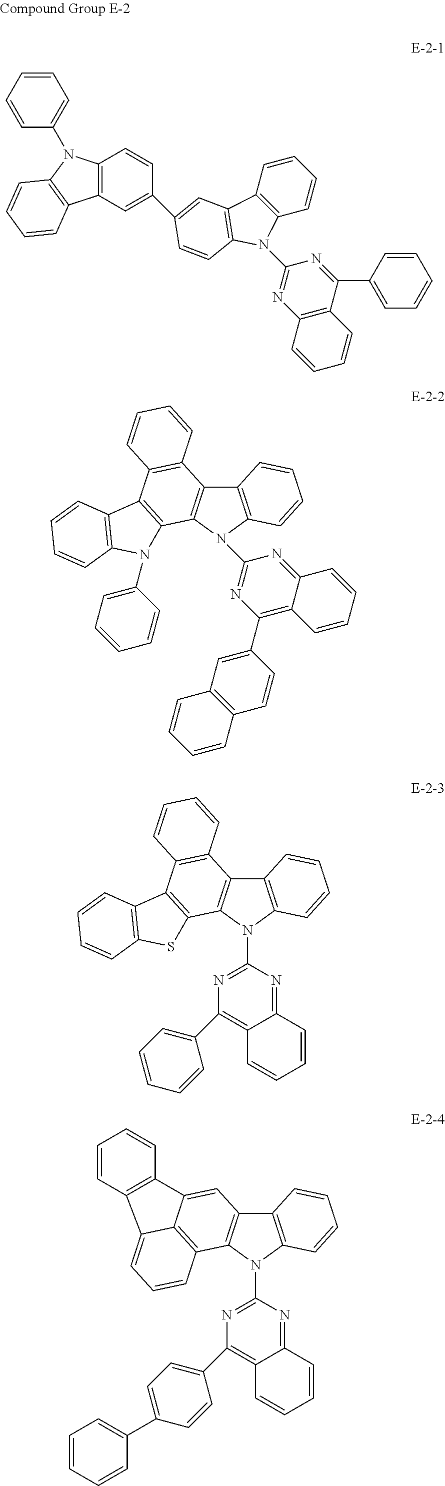

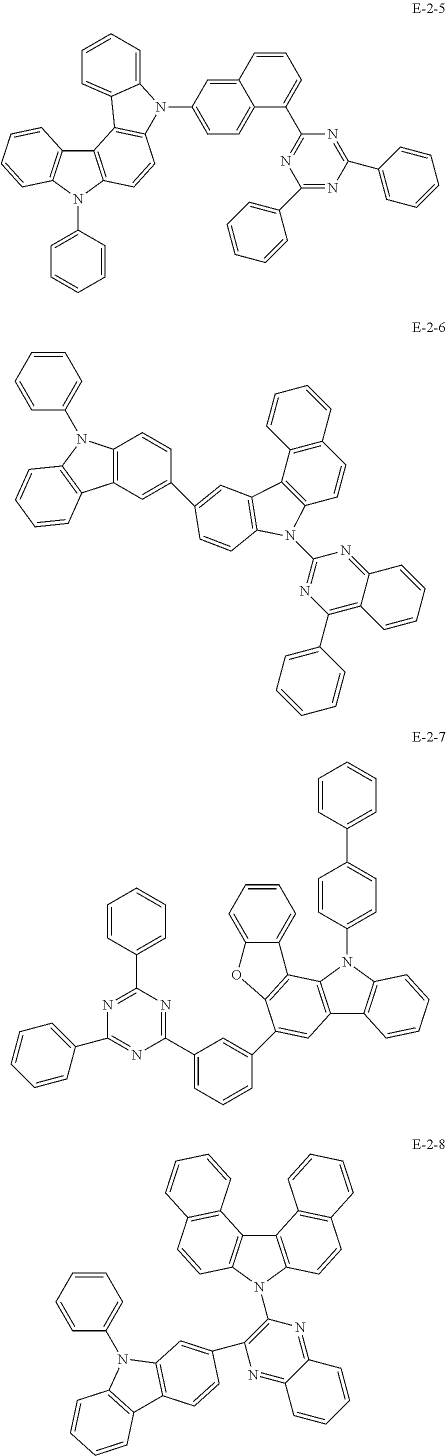 [SMILES: c1ccc(-c2nc(-c3ccccc3)nc(-c3cccc4cc(-n5c6ccccc6c6c7c8ccccc8n(-c8ccccc8)c7ccc65)ccc34)n2)cc1 ; c1ccc(-c2nc(-n3c4ccc(-c5ccc6c(c5)c5ccccc5n6-c5ccccc5)cc4c4c5ccccc5ccc43)nc3ccccc23)cc1 ; c1ccc(-c2ccc(-n3c4ccccc4c4cc(-c5cccc(-c6nc(-c7ccccc7)nc(-c7ccccc7)n6)c5)c5oc6ccccc6c5c43)cc2)cc1 ; c1ccc(-n2c3ccccc3c3ccc(-c4nc5ccccc5nc4-n4c5ccc6ccccc6c5c5c6ccccc6ccc54)cc32)cc1]

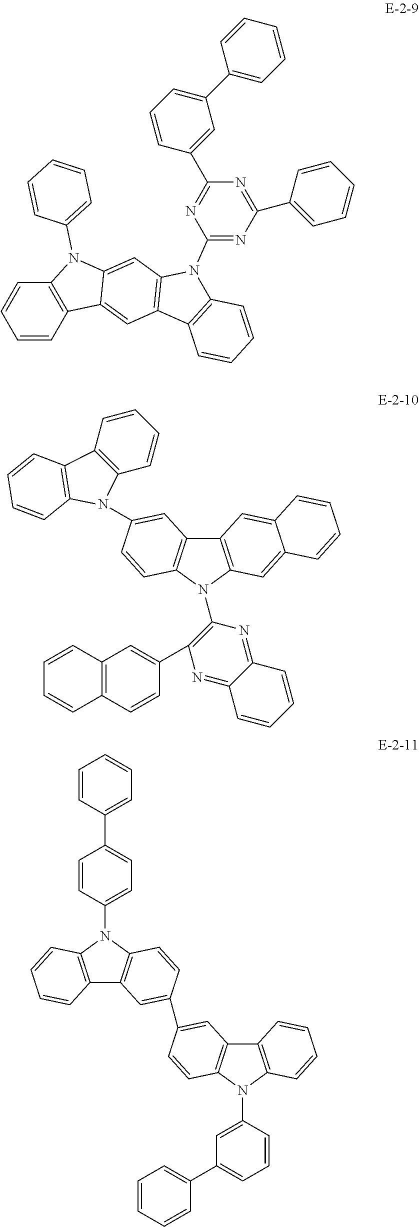 [SMILES: c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc4ccccc4nc3-n3c4ccc(-n5c6ccccc6c6ccccc65)cc4c4cc5ccccc5cc43)ccc2c1 ; c1ccc(-c2ccc(-n3c4ccccc4c4cc(-c5ccc6c(c5)c5ccccc5n6-c5cccc(-c6ccccc6)c5)ccc43)cc2)cc1]

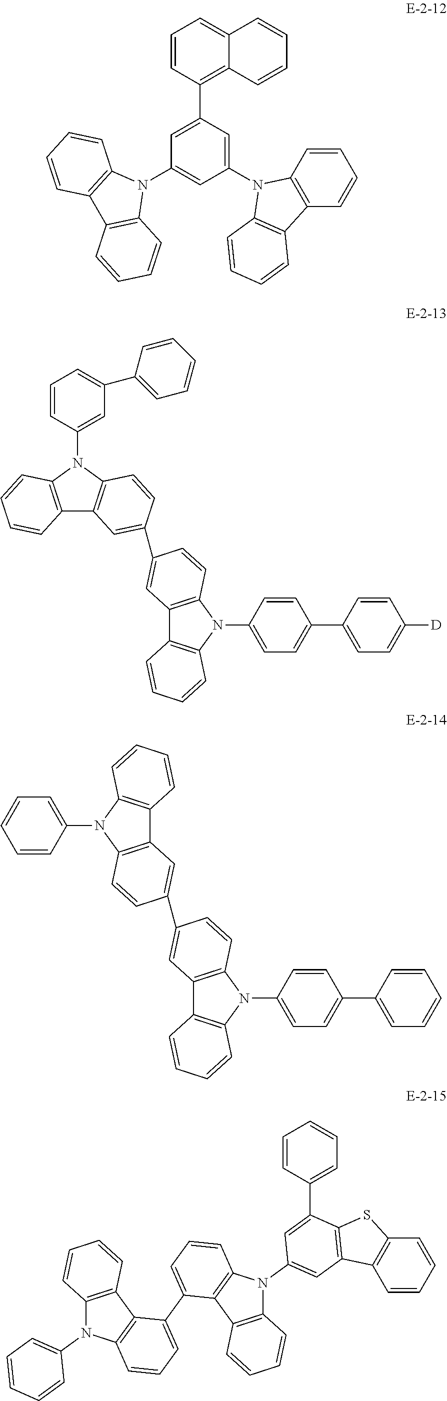 [SMILES: c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1 ; c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1]

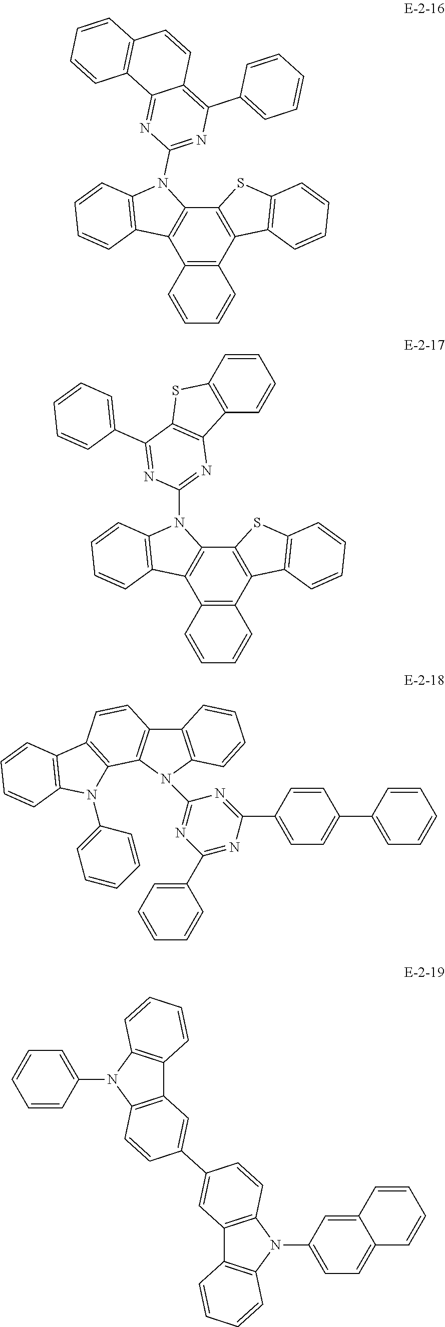

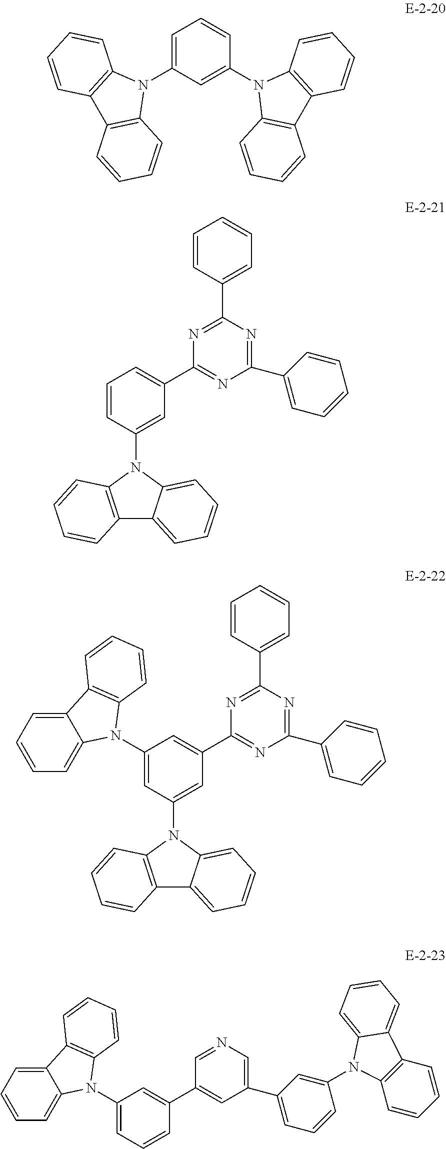 [SMILES: c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1 ; c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1]

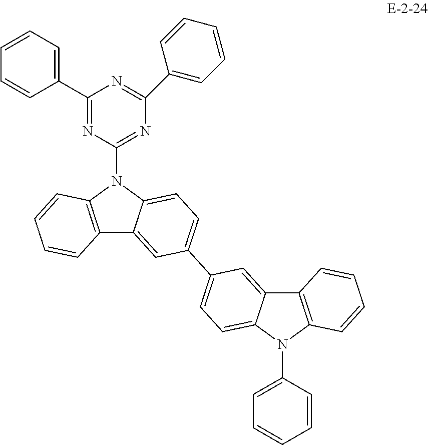 [SMILES: c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a general material suitable in the art as a host material. For example, the emission layer EML may include, as a host material, at least one of bis(4-(9H-carbazol-9-yl)phenyl)diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenylphosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, the embodiment of the disclosure is not limited thereto, for example, tris(8-hydroxyquinolino)aluminum (Alq₃), 9,10-di(naphthalen-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetrasiloxane (DPSiO₄), and/or the like may be used as a host material.

The emission layer EML may include the compound represented by Formula M-a. The compound represented by Formula M-a may be used as a phosphorescent dopant material.

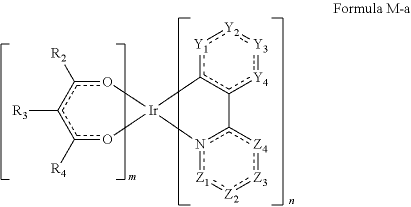

In Formula M-a, Y₁ to Y₄ and Z₁ to Z₄ may each independently be CR₁ or N, R₁ to R₄ may each independently be a hydrogen atom, a deuterium atom, a substituted or unsubstituted amine group, a substituted or unsubstituted thio group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. In Formula M-a, m is 0 or 1, and n is 2 or 3. In Formula M-a, if (e.g., when) m is 0, n is 3, and if (e.g., when) m is 1, n is 2.

The compound represented by Formula M-a may be used as a phosphorescent dopant.

The compound represented by Formula M-a may be represented by any one among (e.g., any one selected from among) Compound M-a1 to Compound M-a25. However, Compounds M-a1 to M-a25 are examples, and the compound represented by Formula M-a is not limited to those represented by Compounds M-a1 to M-a25.

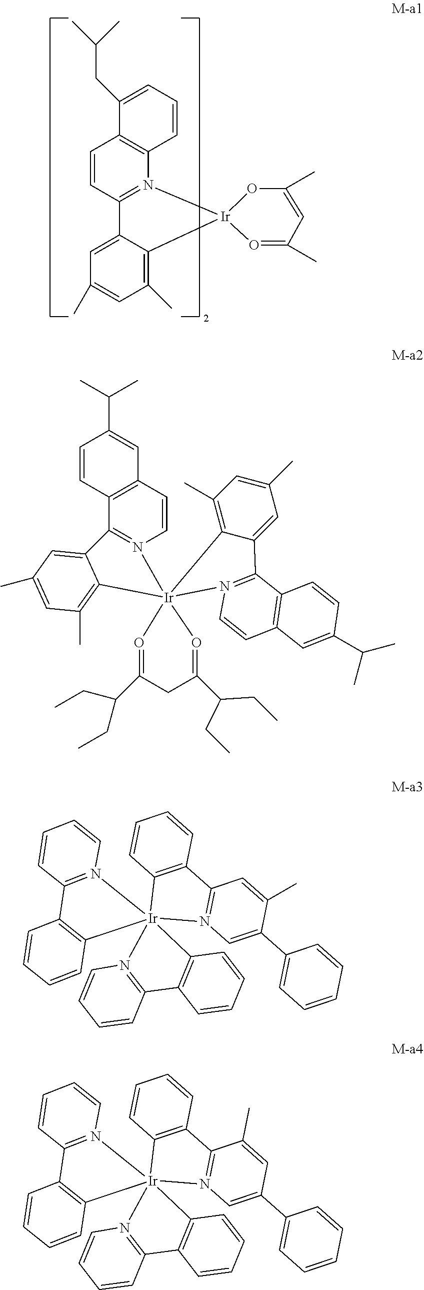

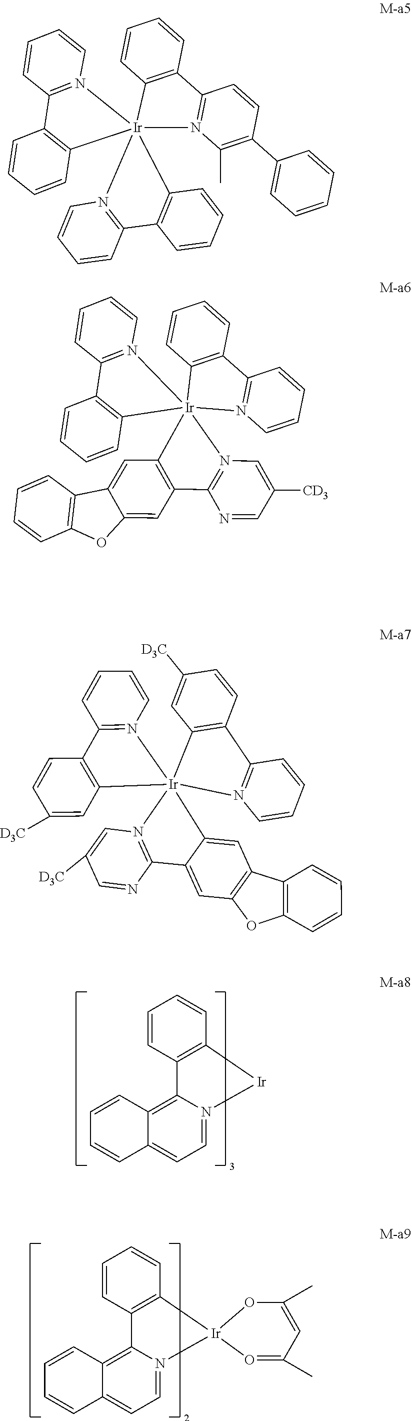

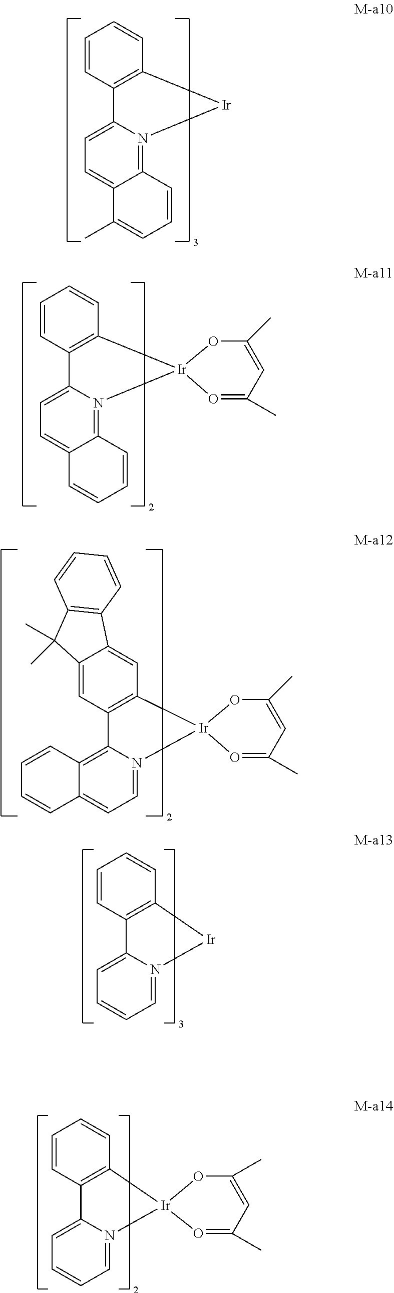

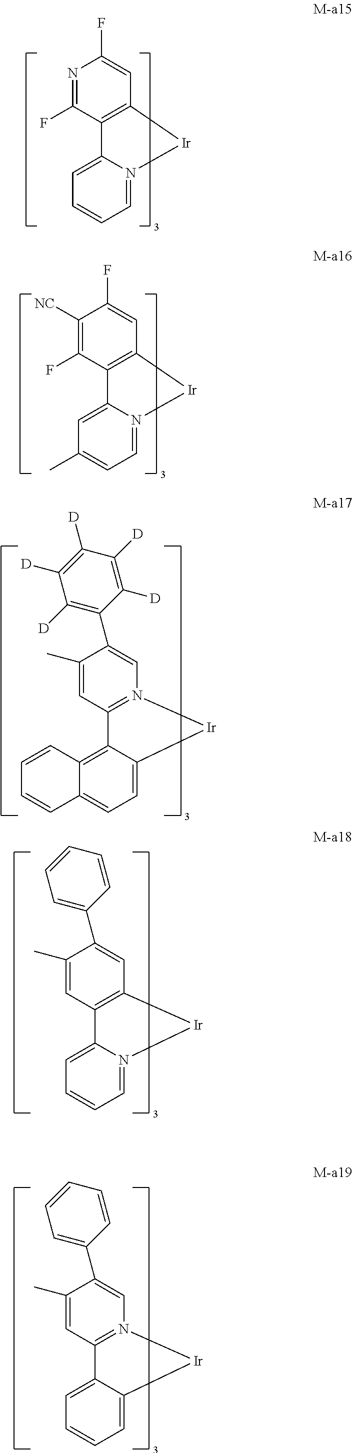

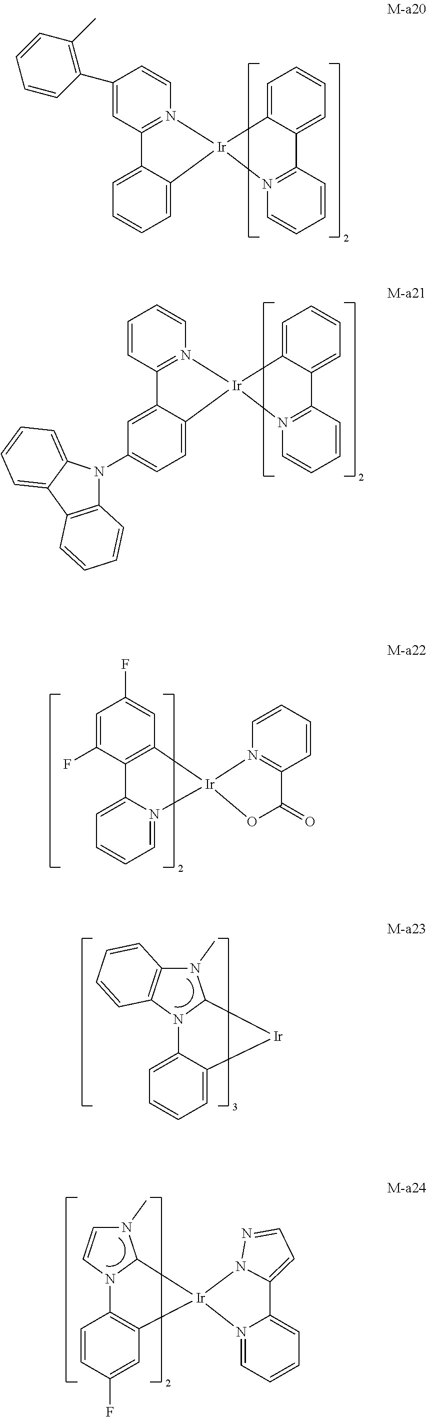

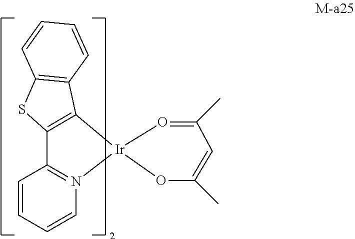

The emission layer EML may include a compound represented by any one among Formula F-a to Formula F-c. The compound represented by Formula F-a to Formula F-c may be used as a fluorescence dopant material.

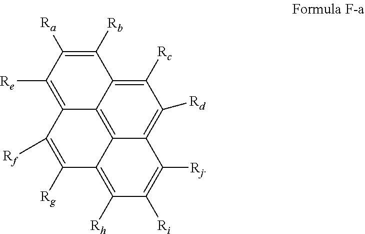

In Formula F-a, two selected from among Rₐ to Rⱼ may each independently be substituted with

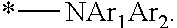

The others, which are not substituted with

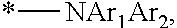

among Rₐ to Rⱼ may each independently be a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atom s.

In

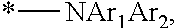

Ar₁ and Ar₂ may each independently be a substituted or unsubstituted aryl group having 6 to 30 ring-forming carbon atoms, or a substituted or unsubstituted heteroaryl group having 2 to 30 ring-forming carbon atoms. For example, at least one of Ar₁ or Ar₂ may be a heteroaryl group containing O or S as a ring-forming atom.

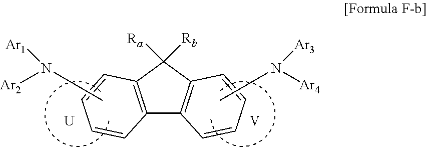

In Formula F-b, Rₐ and R_{b} may each independently be a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 ring-forming carbon atoms, or a substituted or unsubstituted heteroaryl group having 2 to 30 ring-forming carbon atoms, or may be bonded to an adjacent group to form a ring. Ar₁ to Ar₄ may each independently be a substituted or unsubstituted aryl group having 6 to 30 ring-forming carbon atoms, or a substituted or unsubstituted heteroaryl group having 2 to 30 ring-forming carbon atoms.

In Formula F-b, U and V may each independently be a substituted or unsubstituted hydrocarbon ring having 5 to 30 ring-forming carbon atoms, or a substituted or unsubstituted heterocycle having 2 to 30 ring-forming carbon atoms. At least one among Ar₁ to Ar₄ may be a heteroaryl group containing O or S as a ring-forming atom.

In Formula F-b, the number of rings represented by U and V may each independently be 0 or 1. For example, in Formula F-b, it refers to that if (e.g., when) the number of U or V is 1, one ring constitutes a fused ring at a portion indicated by U or V, and if (e.g., when) the number of U or V is 0, a ring indicated by U or V does not exist. For example, if (e.g., when) the number of U is 0 and the number of V is 1, or if (e.g., when) the number of U is 1 and the number of V is 0, the fused ring having a fluorene core in Formula F-b may be a cyclic compound having four rings. In some embodiments, if (e.g., when) each number of U and V is 0, the fused ring in Formula F-b may be a cyclic compound having three rings. In some embodiments, if (e.g., when) each number of U and V is 1, the fused ring having a fluorene core in Formula F-b may be a cyclic compound having five rings.

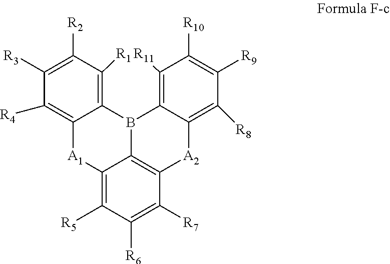

In Formula F-c, A₁ and A₂ may each independently be O, S, Se, or NRₘ, and Rₘ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. R₁ to R₁ may each independently be a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring.

In Formula F-c, A₁ and A₂ may each independently be bonded to substituents of an adjacent ring to form a fused ring. For example, if (e.g., when) A₁ and A₂ may each independently be NRₘ, A₁ may be bonded to R₄ or R₅ to form a ring. In some embodiments, A₂ may be bonded to R₇ or R₈ to form a ring.

In one or more embodiments, the emission layer EML may further include, as a suitable dopant material, a styryl derivative (e.g., 1,4-bis[2-(3-N-ethylcarbazolyl)vinyl]benzene (BCzVB), 4-(di-p-tolylamino)-4′-[(di-p-tolylamino)styryl]stilbene (DPAVB), and N-(4-((E)-2-(6-((E)-4-(diphenylamino)styryl)naphthalen-2-yl)vinyl)phenyl)-N-phenylbenzenamine (N-BDAVBi), 4,4′-bis[2-(4-(N,N-diphenylamino)phenyl)vinyl]biphenyl (DPAVBi), perylene and a derivative thereof (e.g., 2,5,8,11-tetra-t-butylperylene (TBP)), pyrene and a derivative thereof (e.g., 1,1-dipyrene, 1,4-dipyrenylbenzene, 1,4-bis(N,N-diphenylamino)pyrene), and/or the like.

The emission layer EML may further include a suitable phosphorescence dopant material. For example, a metal complex containing iridium (Ir), platinum (Pt), osmium (Os), aurum (Au), titanium (Ti), zirconium (Zr), hafnium (Hf), europium (Eu), terbium (Tb), or thulium (Tm) may be used as a phosphorescent dopant. For example, iridium(III) bis(4,6-difluorophenylpyridinato-N,C2)picolinato (Flrpic), bis(2,4-difluorophenylpyridinato)-tetrakis(1-pyrazolyl)borate iridium(III) (Fir6), or platinum octaethyl porphyrin (PtOEP) may be used as a phosphorescent dopant. However, the embodiment of the disclosure is not limited thereto.

The emission layer EML may include a quantum dot material. The core of the quantum dot may be selected from among a Group II-VI compound, a Group III-VI compound, a Group I-III-VI compound, a Group III-V compound, a Group III-II-V compound, a Group IV-VI compound, a Group IV element, a Group IV compound, and/or a combination thereof.

The Group II-VI compound may be selected from the group consisting of a binary compound selected from the group consisting of CdSe, CdTe, CdS, ZnS, ZnSe, ZnTe, ZnO, HgS, HgSe, HgTe, MgSe, MgS, and a mixture thereof, a ternary compound selected from the group consisting of CdSeS, CdSeTe, CdSTe, ZnSeS, ZnSeTe, ZnTeS, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and a mixture thereof, and a quaternary compound selected from the group consisting of CdZnSeS, CdZnSeTe, CdZnSTe, CdHgSeS, CdHgSeTe, CdHgSTe, HgZnSeS, HgZnSeTe, HgZnSTe, and a mixture thereof. In one or more embodiments, Group II-VI compound may further include a Group I metal and/or IV element. The Group I-II-VI compound may be selected from the group consisting of CuZnS, and/or the like, the Group II-IV-VI compound may be selected from the group consisting of ZnSnS, and/or the like. The Group I-II-IV-VI compound may be selected from among a quaternary compound selected from the group consisting of Cu₂ZnSnS₂, Cu₂ZnSnS₄, Cu₂ZnSnSe₄, Ag₂ZnSnS₂, and a mixture thereof.

The Group III-VI compound may include a binary compound such as In₂S₃ or In₂Se₃, a ternary compound such as InGaS₃ or InGaSe₃, or any combination thereof.

The Group I-III-VI compound may be selected from among a ternary compound selected from the group consisting of AgInS, AgInS₂, CuInS, CuInS₂, AgGaS₂, CuGaS₂, CuGaO₂, AgGaO₂, AgAlO₂, and a mixture thereof, or a quaternary compound such as AgInGaS, AgInGaS₂, AgInGaSe, AgInGaSe₂, CuInGaS, or CuInGaS₂.

The Group III-V compound may be selected from the group consisting of a binary compound selected from the group consisting of GaN, GaP, GaAs, GaSb, AlN, AlP, AlAs, AlSb, InN, InP, InAs, InSb, and a mixture thereof, a ternary compound selected from the group consisting of GaNP, GaNAs, GaNSb, GaPAs, GaPSb, AlNP, AlNAs, AlNSb, AlPAs, AlPSb, InGaP, InAIP, InNP, InNAs, InNSb, InPAs, InPSb, and a mixture thereof, and a quaternary compound selected from the group consisting of GaAlNP, GaAlNAs, GaAlNSb, GaAlPAs, GaAlPSb, GaInNP, GaInNAs, GaInNSb, GaInPAs, GaInPSb, InAlNP, InAlNAs, InAlNSb, InAlPAs, InAlPSb, and a mixture thereof. In one or more embodiments, the Group III-V compound may further include a Group II metal. For example, InZnP, and/or the like, may be selected as a Group III-II-V compound.

The Group IV-VI compound may be selected from the group consisting of a binary compound selected from the group consisting of SnS, SnSe, SnTe, PbS, PbSe, PbTe, and a mixture thereof, a ternary compound selected from the group consisting of SnSeS, SnSeTe, SnSTe, PbSeS, PbSeTe, PbSTe, SnPbS, SnPbSe, SnPbTe, and a mixture thereof, and a quaternary compound selected from the group consisting of SnPbSSe, SnPbSeTe, SnPbSTe, and a mixture thereof. The Group IV element may be selected from the group consisting of Si, Ge, and a mixture thereof. The Group IV compound may be a binary compound selected from the group consisting of SiC, SiGe, and a mixture thereof.

Each element included in a polynary compound such as the binary compound, the ternary compound, or the quaternary compound may be present in a particle with a substantially uniform or non-substantially uniform concentration distribution. For example, the formulae refer to the types (kinds) of elements included in the compounds, and the elemental ratio in the compound may be different. For example, AgInGaS₂ may refer to AgInₓGa₁₋ₓS₂ (where x is a real number of 0 to 1).

In one or more embodiments, the quantum dot may have a single structure or a double structure of core-shell in which the concentration of each element included in the quantum dot is substantially uniform. For example, the material included in the core may be different from the material included in the shell.

The shell of the quantum dot may serve as a protection layer to prevent or reduce the chemical deformation of the core to maintain semiconductor properties, and/or a charging layer to impart electrophoresis properties to the quantum dot. The shell may be a single layer or multiple layers. An interface between the core and the shell may have a concentration gradient in which the concentration of an element present in the shell becomes lower towards the center.

An example of the shell of the quantum dots may include a metal or non-metal oxide, a semiconductor compound, or a combination thereof. For example, the metal or non-metal oxide may be a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄, or NiO, or a ternary compound such as MgAl₂O₄, CoFe₂O₄, NiFe₂O₄, or CoMn₂O₄, but the embodiment of the disclosure is not limited thereto.

Also, examples of the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AIP, AlSb, and/or the like, but the embodiment of the disclosure is not limited thereto.

Each element included in a polynary compound such as the binary compound, or the ternary compound may be present in a particle with a substantially uniform or non-substantially uniform concentration distribution. For example, the formulae refer to the types (kinds) of elements included in the compounds, and the elemental ratio in the compound may be different.

The quantum dot may have a full width of half maximum (FWHM) of a light emitting wavelength spectrum of about 45 nanometer (nm) or less, about 40 nm or less, or about 30 nm or less, and color purity or color reproducibility may be improved in the above range. In some embodiments, light emitted through such quantum dot is emitted in all directions so that a wide viewing angle may be improved.

In some embodiments, although the form of the quantum dot is not particularly limited as long as it is a form commonly used in the art, more specifically, the quantum dot in the form of spherical, pyramidal, multi-arm, or cubic nanoparticles, nanotubes, nanowires, nanofibers, nanoplate particles, and/or the like. may be used.

As the size of the quantum dot is adjusted or the elemental ratio in the quantum dot compound is adjusted, it is possible to control the energy band gap, and thus light in one or more suitable wavelength ranges may be obtained in the quantum dot emission layer. Therefore, the quantum dot as above (using different sizes of quantum dots or different elemental ratios in the quantum dot compound) is used, and thus the light emitting element, which emits light in one or more suitable wavelengths, may be implemented. For example, the adjustment of the size of the quantum dot or the elemental ratio in the quantum dot compound may be selected to emit red, green, and/or blue light. In some embodiments, the quantum dots may be configured to emit white light by combining one or more suitable colors of light.

In each of the light emitting elements ED of embodiments illustrated in FIGS. 5 to 8, the electron transport region ETR is provided on the emission layer EML. The electron transport region ETR may include at least one of the hole blocking layer HBL, the electron transport layer ETL, or the electron injection layer EIL, but the embodiment of the disclosure is not limited thereto.

The electron transport region ETR may have a single layer formed of a single material, a single layer formed of a plurality of different materials, or a multilayer structure including a plurality of layers formed of a plurality of different materials.

For example, the electron transport region ETR may have a single layer structure of the electron injection layer EIL or the electron transport layer ETL, and may have a single layer structure formed of an electron injection material and an electron transport material. In some embodiments, the electron transport region ETR may have a single layer structure formed of a plurality of different materials, or may have a structure in which an electron transport layer ETL/electron injection layer EIL, a hole blocking layer HBL/electron transport layer ETL/electron injection layer EIL are stacked in order from the emission layer EML, but the embodiment of the disclosure is not limited thereto. The electron transport region ETR may have a thickness, for example, from about 1,000 Å to about 1,500 Å.

The electron transport region ETR may be formed using one or more suitable methods such as a vacuum deposition method, a spin coating method, a cast method, a Langmuir-Blodgett (LB) method, an inkjet printing method, a laser printing method, and a laser induced thermal imaging (LITI) method.

The electron transport region ETR may include a compound represented by Formula ET-2:

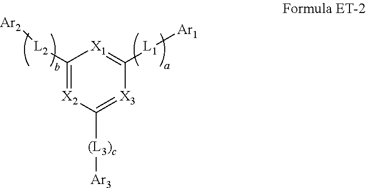

In Formula ET-2, at least one among X₁ to X₃ is N, and the rest are CRₐ. Rₐ may be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms. Ar₁ to Ar₃ may each independently be a hydrogen atom, a deuterium atom, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

In Formula ET-2, a to c may each independently be an integer of 0 to 10. In Formula ET-2, L₁ to L₃ may each independently be a direct linkage, a substituted or unsubstituted arylene group having 6 to 30 ring-forming carbon atoms, or a substituted or unsubstituted heteroarylene group having 2 to 30 ring-forming carbon atoms. In one or more embodiments, if (e.g., when) a to c may each independently be an integer of 2 or more, L₁ to L₃ may each independently be a substituted or unsubstituted arylene group having 6 to 30 ring-forming carbon atoms, or a substituted or unsubstituted heteroarylene group having 2 to 30 ring-forming carbon atoms.

The electron transport region ETR may include an anthracene-based compound. However, the embodiment of the disclosure is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazol-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1, 10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), beryllium bis(benzoquinolin-10-olate) (Bebq₂), 9,10-di(naphthalen-2-yl)anthracene (ADN), 1,3-bis[3,5-di(pyridin-3-yl)phenyl]benzene (BmPyPhB), or a mixture thereof.

The electron transport region ETR may include at least one among (e.g., at least one selected from among) Compound ET1 to Compound ET36:

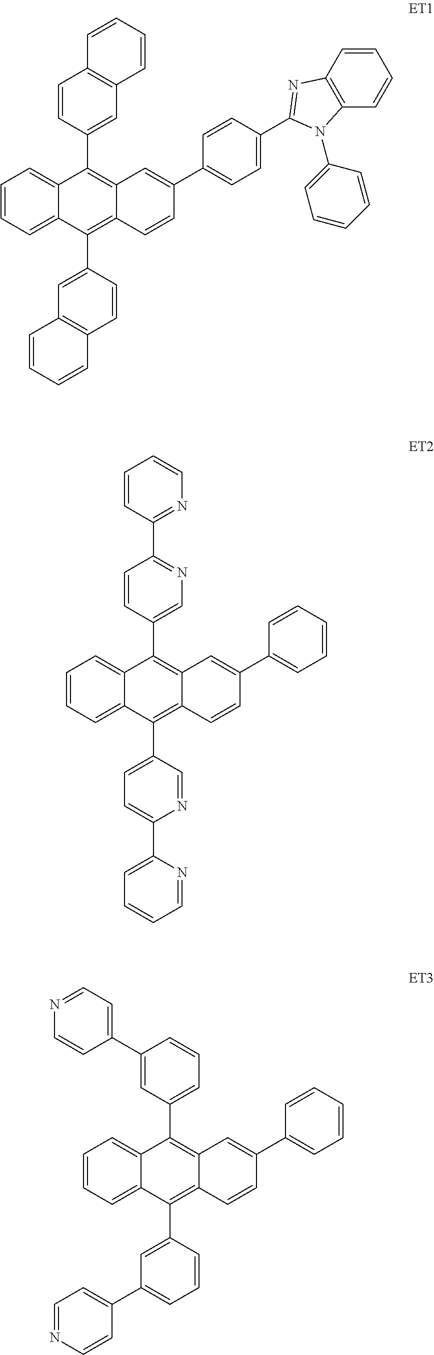 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

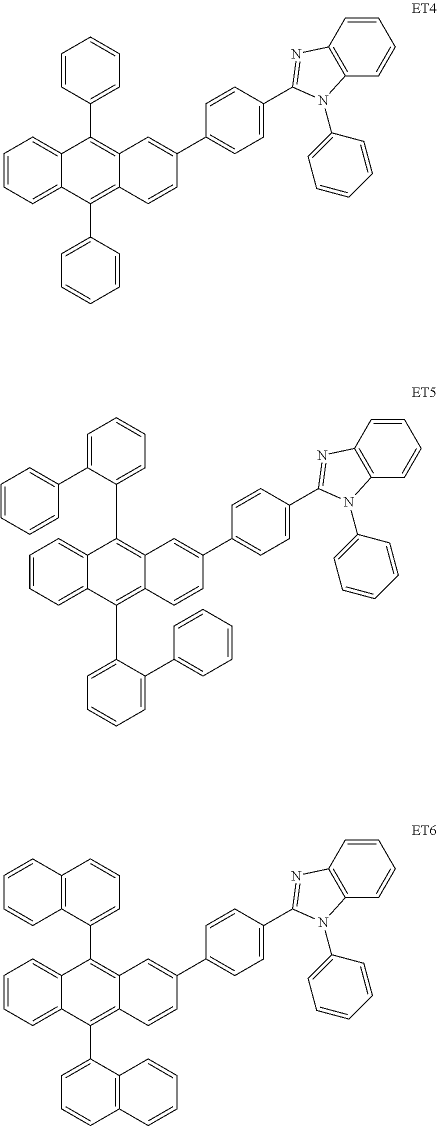 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1]

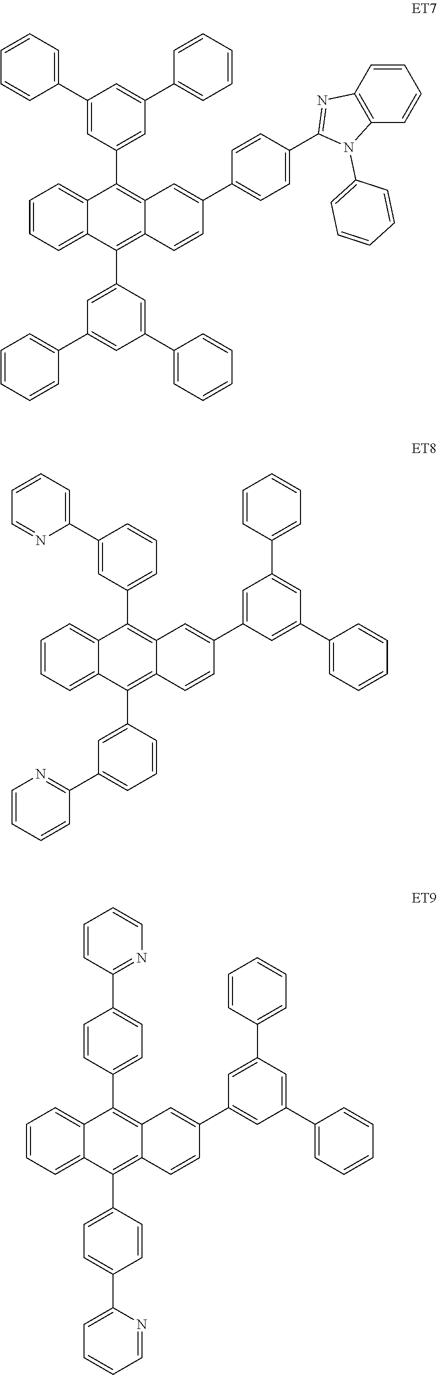 [SMILES: c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1 ; c1ccc(-c2cc(-c3ccccc3)cc(-c3ccc4c(-c5cccc(-c6ccccn6)c5)c5ccccc5c(-c5cccc(-c6ccccn6)c5)c4c3)c2)cc1 ; c1ccc(-c2cc(-c3ccccc3)cc(-c3ccc4c(-c5ccc(-c6ccccn6)cc5)c5ccccc5c(-c5ccc(-c6ccccn6)cc5)c4c3)c2)cc1]

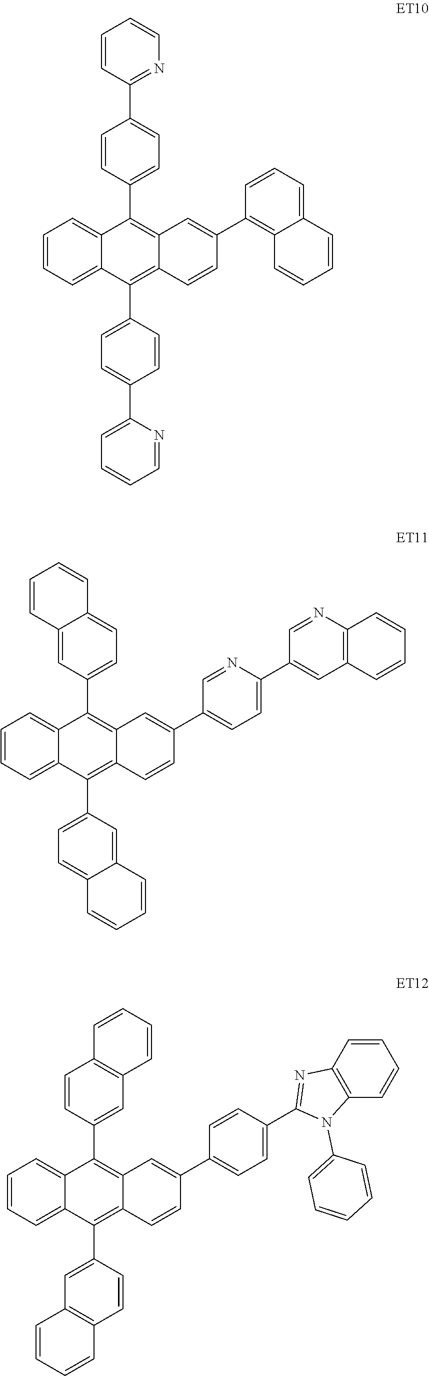 [SMILES: c1ccc(-c2ccc(-c3c4ccccc4c(-c4ccc(-c5ccccn5)cc4)c4cc(-c5cccc6ccccc56)ccc34)cc2)nc1 ; c1ccc2cc(-c3c4ccccc4c(-c4ccc5ccccc5c4)c4cc(-c5ccc(-c6cnc7ccccc7c6)nc5)ccc34)ccc2c1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1]

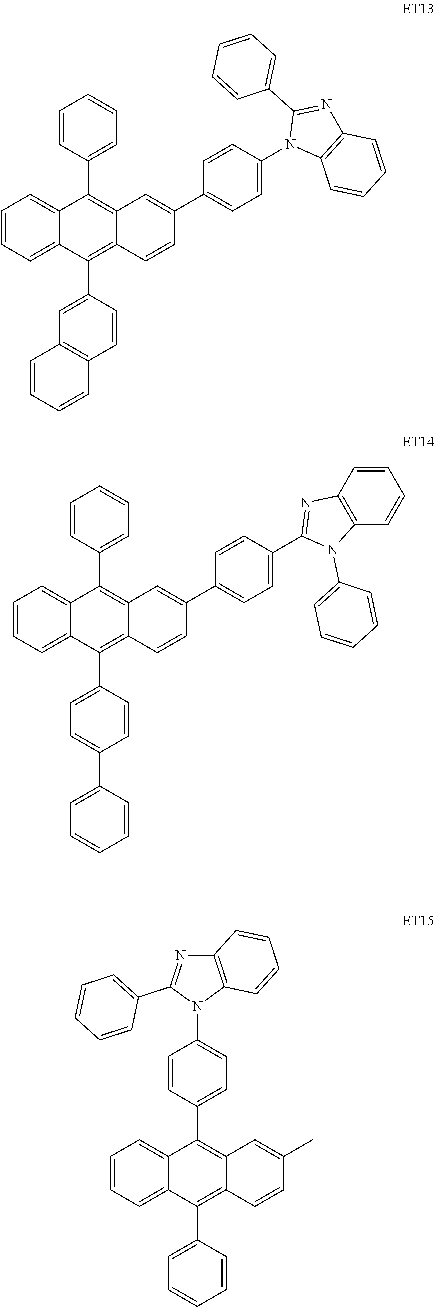 [SMILES: c1ccc(-c2c3ccccc3c(-c3ccc4ccccc4c3)c3ccc(-c4ccc(-n5c(-c6ccccc6)nc6ccccc65)cc4)cc23)cc1 ; c1ccc(-c2ccc(-c3c4ccccc4c(-c4ccccc4)c4cc(-c5ccc(-c6nc7ccccc7n6-c6ccccc6)cc5)ccc34)cc2)cc1 ; Cc1ccc2c(-c3ccccc3)c3ccccc3c(-c3ccc(-n4c(-c5ccccc5)nc5ccccc54)cc3)c2c1]

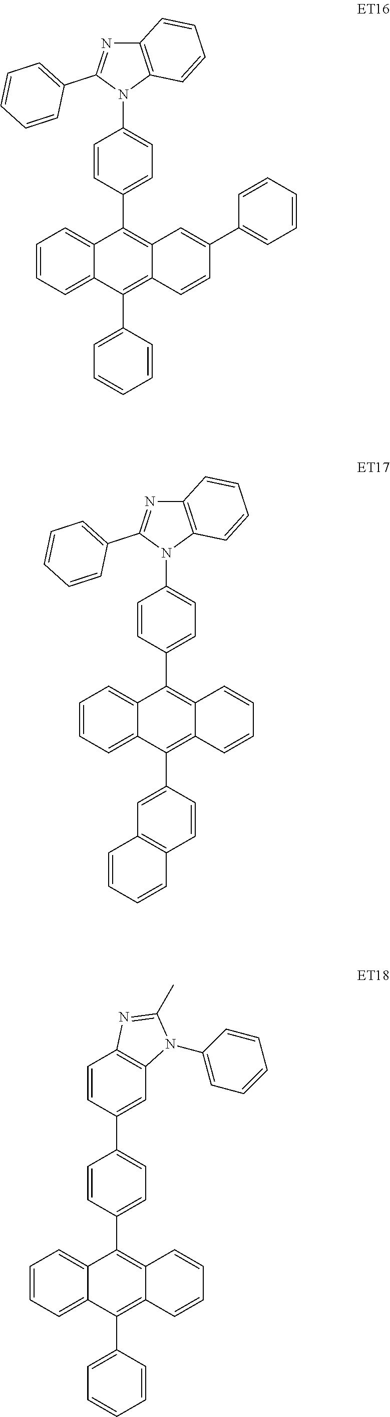 [SMILES: c1ccc(-c2ccc3c(-c4ccccc4)c4ccccc4c(-c4ccc(-n5c(-c6ccccc6)nc6ccccc65)cc4)c3c2)cc1 ; c1ccc(-c2nc3ccccc3n2-c2ccc(-c3c4ccccc4c(-c4ccc5ccccc5c4)c4ccccc34)cc2)cc1 ; Cc1nc2ccc(-c3ccc(-c4c5ccccc5c(-c5ccccc5)c5ccccc45)cc3)cc2n1-c1ccccc1]

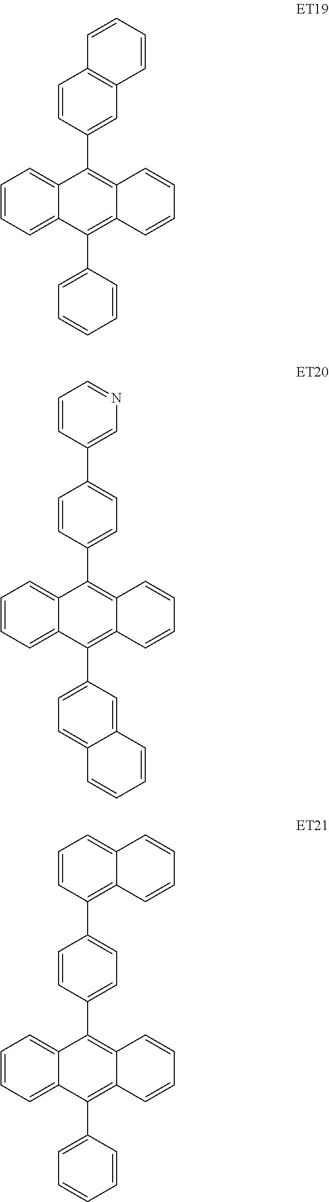

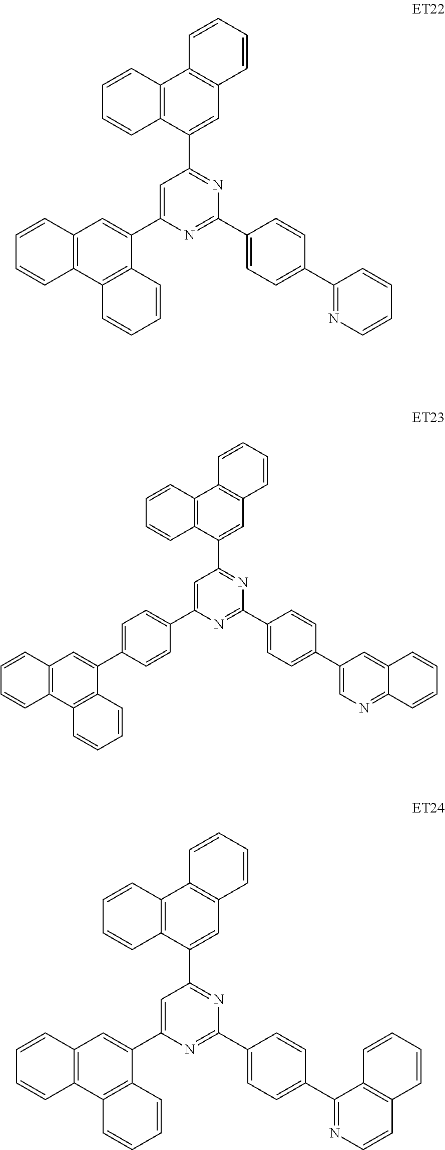 [SMILES: c1ccc(-c2ccc(-c3nc(-c4cc5ccccc5c5ccccc45)cc(-c4cc5ccccc5c5ccccc45)n3)cc2)nc1 ; c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1]

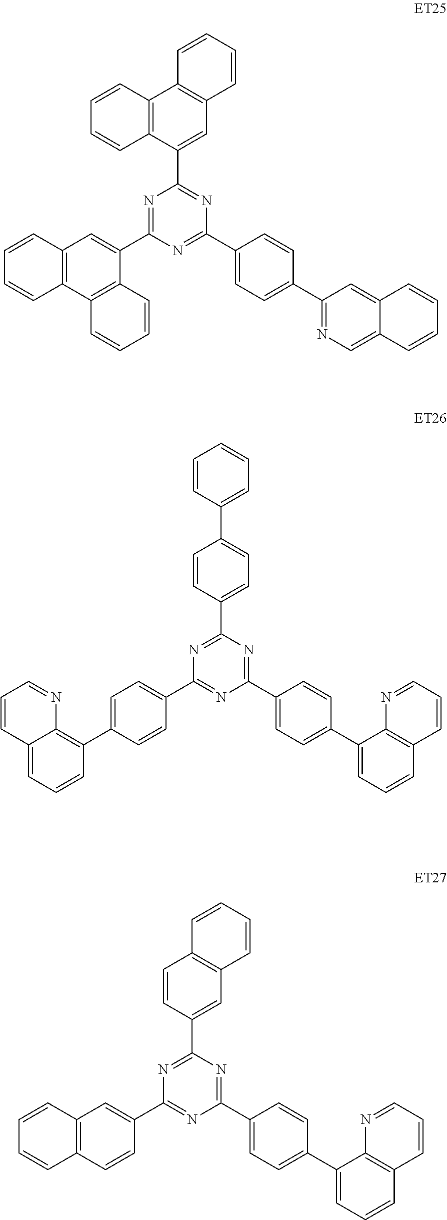 [SMILES: c1ccc2cc(-c3ccc(-c4nc(-c5cc6ccccc6c6ccccc56)nc(-c5cc6ccccc6c6ccccc56)n4)cc3)ncc2c1 ; c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1]

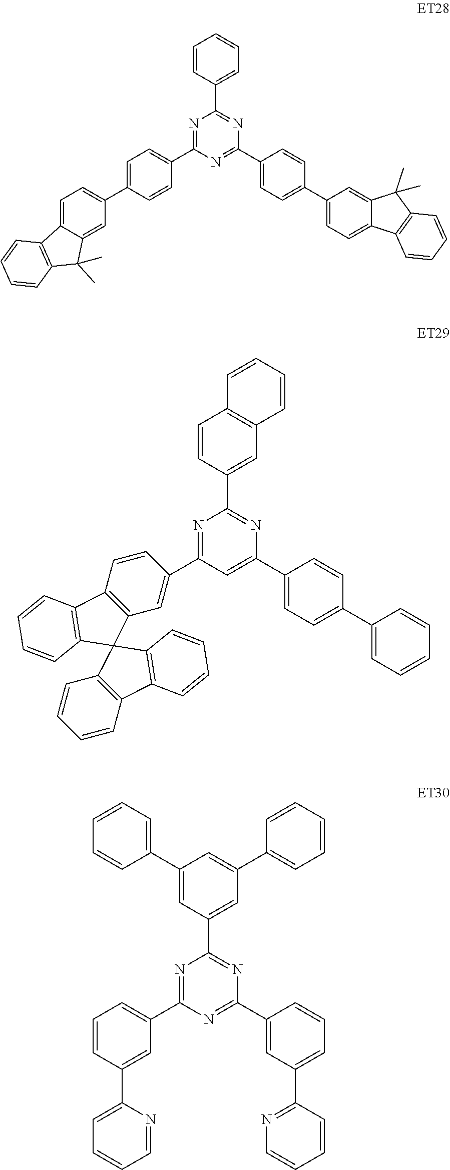 [SMILES: CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21 ; c1ccc(-c2ccc(-c3cc(-c4ccc5c(c4)C4(c6ccccc6-c6ccccc64)c4ccccc4-5)nc(-c4ccc5ccccc5c4)n3)cc2)cc1 ; c1ccc(-c2cc(-c3ccccc3)cc(-c3nc(-c4cccc(-c5ccccn5)c4)nc(-c4cccc(-c5ccccn5)c4)n3)c2)cc1]

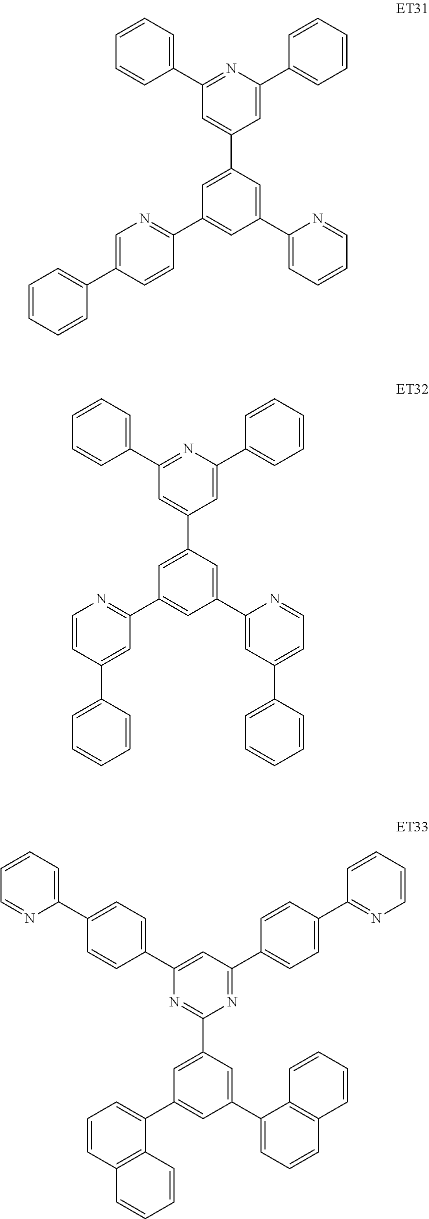 [SMILES: c1ccc(-c2ccc(-c3cc(-c4cc(-c5ccccc5)nc(-c5ccccc5)c4)cc(-c4ccccn4)c3)nc2)cc1 ; c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1]

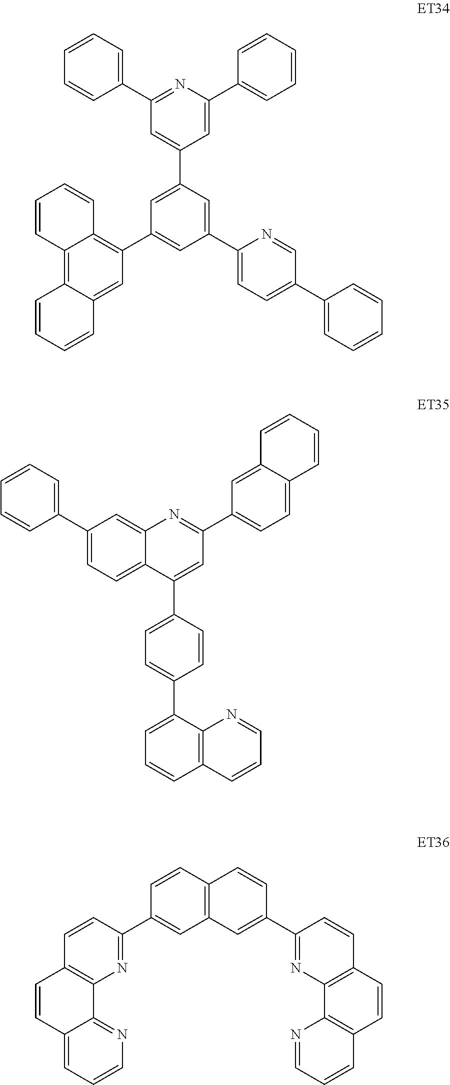 [SMILES: c1ccc(-c2ccc(-c3cc(-c4cc(-c5ccccc5)nc(-c5ccccc5)c4)cc(-c4cc5ccccc5c5ccccc45)c3)nc2)cc1 ; c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In some embodiments, the electron transport region ETR may include a metal halide such as LiF, NaCl, CsF, RbCl, RbI, CuI, and KI, a lanthanide metal such as Yb, and a co-deposited material of the metal halide and the lanthanide metal. For example, the electron transport region ETR may include KI:Yb, RbI:Yb, LiF:Yb, and/or the like, as a co-deposited material. In one or more embodiments, the electron transport region ETR may be formed using a metal oxide such as Li₂O or BaO, or 8-hydroxyl-lithium quinolate (Liq), and/or the like, but the embodiment of the disclosure is not limited thereto. The electron transport region ETR may also be formed of a mixture material of an electron transport material and an insulating organometallic salt. The organometallic salt may be a material having an energy band gap of about 4 eV or more. For example, the organometallic salt may include, for example, a metal acetate, a metal benzoate, a metal acetoacetate, a metal acetylacetonate, or a metal stearate.

The electron transport region ETR may further include at least one of 2,9-dimethyl-4,7-diphenyl-1, 10-phenanthroline (BCP), diphenyl(4-(triphenylsilyl)phenyl)phosphine oxide (TSPO1), or 4,7-diphenyl-1, 10-phenanthroline (Bphen) in addition to the herein-described materials, but the embodiment of the disclosure is not limited thereto.

The electron transport region ETR may include the herein-described compounds of the hole transport region in at least one of the electron injection layer EIL, the electron transport layer ETL, or the hole blocking layer HBL.

When the electron transport region ETR includes the electron transport layer ETL, the electron transport layer ETL may have a thickness of about 100 Å to about 1,000 Å, for example, about 150 Å to about 500 Å. If the thickness of the electron transport layer ETL satisfies the aforementioned range, satisfactory electron transport characteristics may be obtained without a substantial increase in driving voltage. When the electron transport region ETR includes the electron injection layer EIL, the electron injection layer EIL may have a thickness of about 1 Å to about 100 Å, for example, about 3 Å to about 90 Å. If the thickness of the electron injection layer EIL satisfies the herein-described range, satisfactory electron injection characteristics may be obtained without a substantial increase in driving voltage.

The second electrode EL2 is provided on the electron transport region ETR. The second electrode EL2 may be a common electrode. The second electrode EL2 may be a cathode or an anode, but the embodiment of the disclosure is not limited thereto. For example, if (e.g., when) the first electrode EL1 is an anode, the second electrode EL2 may be a cathode, and if (e.g., when) the first electrode EL1 is a cathode, the second electrode EL2 may be an anode.

The second electrode EL2 may be a transmissive electrode, a transflective electrode, or a reflective electrode. When the second electrode EL2 is the transmissive electrode, the second electrode EL2 may be formed of a transparent metal oxide, for example, indium tin oxide (ITO), indium zinc oxide (IZO), zinc oxide (ZnO), indium tin zinc oxide (ITZO), and/or the like.

When the second electrode EL2 is the transflective electrode or the reflective electrode, the second electrode EL2 may include Ag, Mg, Cu, Al, Pt, Pd, Au, Ni, Nd, Ir, Cr, Li, Ca, LiF/Ca, LiF/Al, Mo, Ti, Yb, W, or a compound or mixture thereof (e.g., AgMg, AgYb, or MgYb). In one or more embodiments, the second electrode EL2 may have a multilayer structure including a reflective film or a transflective film formed of the herein-described materials, and a transparent conductive film formed of ITO, IZO, ZnO, ITZO, and/or the like. For example, the second electrode EL2 may include the herein-described metal materials, combinations of at least two metal materials of the herein-described metal materials, oxides of the herein-described metal materials, and/or the like.

The second electrode EL2 may be connected with an auxiliary electrode. If the second electrode EL2 is connected with the auxiliary electrode, the resistance of the second electrode EL2 may be decreased.

In one or more embodiments, a capping layer CPL may further be arranged on the second electrode EL2 of the light emitting element ED of one or more embodiments. The capping layer CPL may include a multilayer or a single layer.

In one or more embodiments, the capping layer CPL may be an organic layer or an inorganic layer. For example, if (e.g., when) the capping layer CPL contains an inorganic material, the inorganic material may include an alkaline metal compound (e.g., LiF), an alkaline earth metal compound (e.g., MgF₂), SiON, SiNₓ, SiOy, and/or the like.

For example, if (e.g., when) the capping layer CPL includes an organic material, the organic material may include α-NPD, NPB, TPD, m-MTDATA, Alq₃, CuPc, N4,N4,N4′,N4′-tetra(biphenyl-4-yl)biphenyl-4,4′-diamine (TPD15), 4,4′,4″-tris(carbazol-9-yl)triphenylamine (TCTA), and/or the like, or an epoxy resin, or acrylate such as methacrylate. However, the embodiment of the disclosure is not limited thereto, and the capping layer CPL may include at least one among (e.g., at least one selected from among) Compounds P1 to P5:

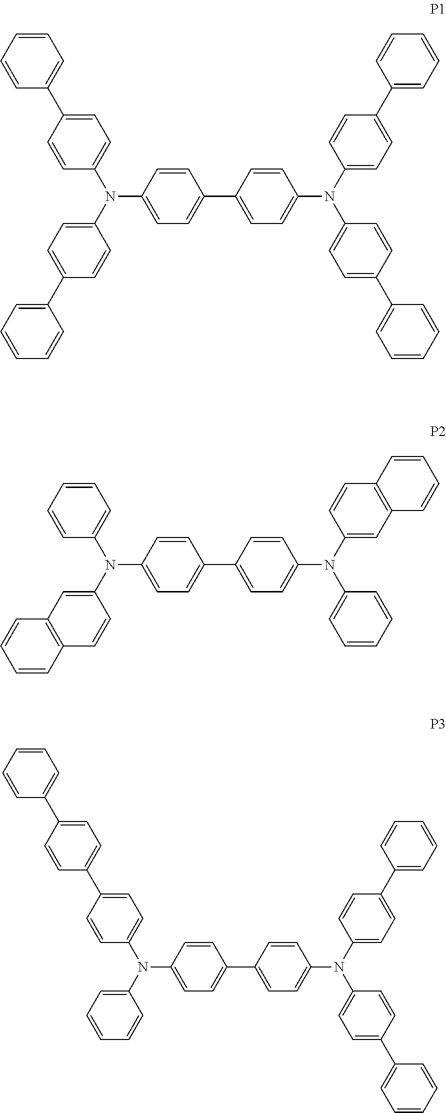 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

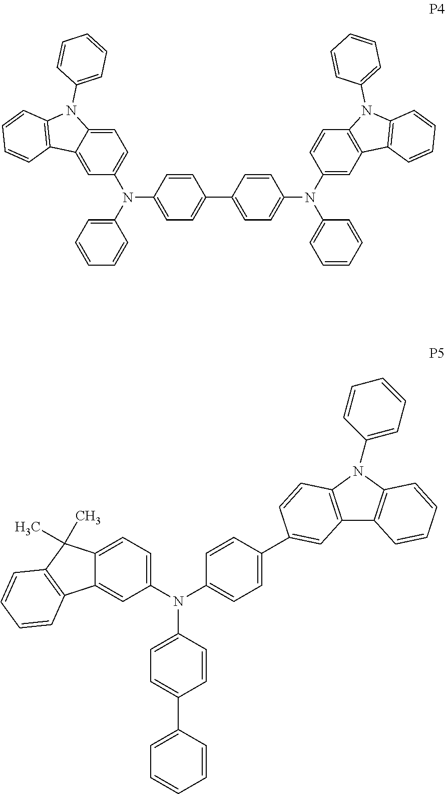 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

In one or more embodiments, the refractive index of the capping layer CPL may be about 1.6 or more. For example, the refractive index of the capping layer CPL may be about 1.6 or more with respect to light in a wavelength range of about 550 nm to about 660 nm.

In the light emitting element ED, if (e.g., when) voltage is applied to the first electrode EL1 and the second electrode EL2, holes injected from the first electrode EL1 move to the emitting layer EML through the hole transport region HTR, and electrons injected from the second electrode EL2 move to the emitting layer EML through the electron transport region ETR. Electrons and holes recombine in the emitting layer EML to generate excitons, and light is emitted if (e.g., when) the excitons drop from the excited state to the ground state.

The nitrogen-containing compound according to one or more embodiments of the disclosure includes a core of a six-membered ring containing at least one nitrogen atom as a ring-forming atom, and has a structure in which two heterocycle substituents represented by any one of Formulas S-1 and S-2 are connected to the core through a linker of a heteroarylene group having 6 or more ring-forming carbon atoms, and may thus exhibit excellent or suitable electron transport properties.

The light emitting element ED of one or more embodiments uses the nitrogen-containing compound of one or more embodiments as a host in the emission layer EML to emit blue thermally activated delayed light or blue phosphorescent light, and may exhibit high efficiency and long service life characteristics.

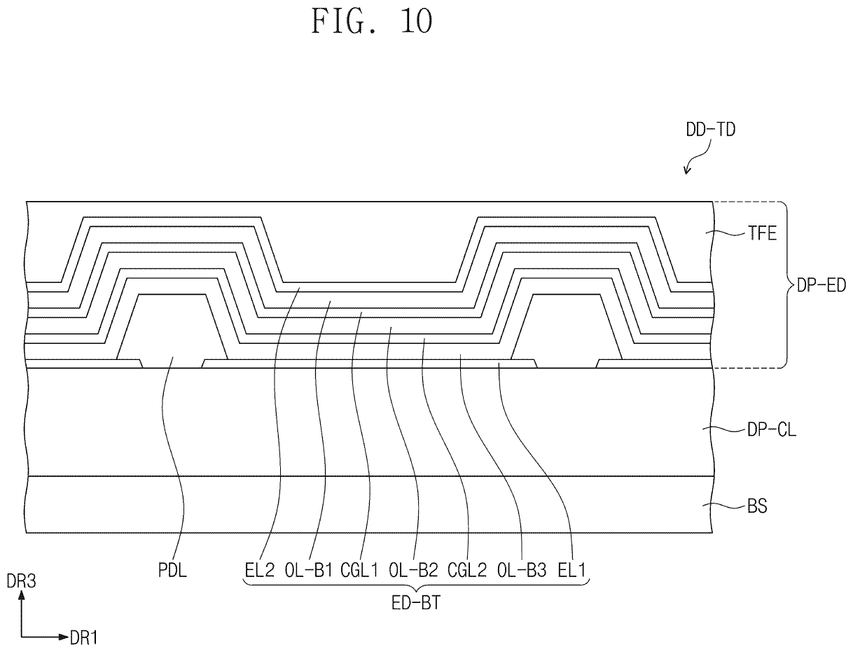

Each of FIGS. 9 to 12 is a cross-sectional view of a display device according to one or more embodiments of the disclosure. Hereinafter, in describing the display devices of embodiments with reference to FIGS. 9 to 12, the duplicated features which have been described in FIGS. 3 to 8 are not described again, but their differences will be mainly described.

Referring to FIG. 9, the display device DD-a according to one or more embodiments may include a display panel DP including a display device layer DP-ED, a light control layer CCL arranged on the display panel DP, and a color filter layer CFL. In one or more embodiments illustrated in FIG. 9, the display panel DP may include a base layer BS, a circuit layer DP-CL provided on the base layer BS, and the display device layer DP-ED, and the display device layer DP-ED may include a light emitting element ED.

The light emitting element ED may include a first electrode EL1, a hole transport region HTR arranged on the first electrode EL1, an emission layer EML arranged on the hole transport region HTR, an electron transport region ETR arranged on the emission layer EML, and a second electrode EL2 arranged on the electron transport region ETR. In one or more embodiments, the structures of the light emitting elements of FIGS. 5 to 8 as described herein may be equally applied to the structure of the light emitting element ED illustrated in FIG. 9.

The emission layer EML of the light emitting element ED included in the display device DD-a according to one or more embodiments may include the nitrogen-containing compound of one or more embodiments described herein. The light emitting element ED of one or more embodiments exhibits excellent or suitable efficiency and long life characteristics, including the nitrogen-containing compound of one or more embodiments in the emission layer EML, and accordingly, the display device DD-a of one or more embodiments may exhibit excellent or suitable display quality.

Referring to FIG. 9, the emission layer EML may be arranged in an opening OH defined in a pixel defining film PDL. For example, the emission layer EML which is divided (e.g., defined) by the pixel defining film PDL and provided corresponding to each light emitting regions PXA-R, PXA-G, and PXA-B may be to emit light in substantially the same wavelength range. In the display device DD-a of one or more embodiments, the emission layer EML may be to emit blue light. In one or more embodiments, unlike the configuration illustrated, in one or more embodiments, the emission layer EML may be provided as a common layer in the entire light emitting regions PXA-R, PXA-G, and PXA-B.

The light control layer CCL may be arranged on the display panel DP. The light control layer CCL may include a light conversion body. The light conversion body may be a quantum dot, a phosphor, and/or the like. The light conversion body may be to emit provided light by converting the wavelength thereof. For example, the light control layer CCL may a layer containing the quantum dot or a layer containing the phosphor.

The light control layer CCL may include a plurality of light control parts CCP1, CCP2 and CCP3. The light control parts CCP1, CCP2, and CCP3 may be spaced and/or apart (e.g., spaced apart or separated) from each other.

Referring to FIG. 9, divided (e.g., defined) patterns BMP may be arranged between the light control parts CCP1, CCP2 and CCP3 which are spaced and/or apart (e.g., spaced apart or separated) from each other, but the embodiment of the disclosure is not limited thereto. FIG. 9 illustrates that the divided (e.g., defined) patterns BMP do not overlap the light control parts CCP1, CCP2 and CCP3, but at least a portion of the edges of the light control parts CCP1, CCP2 and CCP3 may overlap the divided (e.g., defined) patterns BMP.

The light control layer CCL may include a first light control part CCP1 containing a first quantum dot QD1 which converts first color light provided from the light emitting element ED into second color light, a second light control part CCP2 containing a second quantum dot QD2 which converts the first color light into third color light, and a third light control part CCP3 which transmits the first color light. In one or more embodiments, the first light control part CCP1 may provide red light that is the second color light, and the second light control part CCP2 may provide green light that is the third color light. The third light control part CCP3 may provide blue light by transmitting the blue light that is the first color light provided from the light emitting element ED. For example, the first quantum dot QD1 may be a red quantum dot, and the second quantum dot QD2 may be a green quantum dot. The same as described herein may be applied with respect to the quantum dots QD1 and QD2.

In some embodiments, the light control layer CCL may further include a scatterer SP. The first light control part CCP1 may include the first quantum dot QD1 and the scatterer SP, the second light control part CCP2 may include the second quantum dot QD2 and the scatterer SP, and the third light control part CCP3 may not include (e.g., may exclude) any quantum dot but include the scatterer SP.

The scatterer SP may be inorganic particles. For example, the scatterer SP may include at least one of TiO₂, ZnO, Al₂O₃, SiO₂, or hollow sphere silica. The scatterer SP may include any one among TiO₂, ZnO, Al₂O₃, SiO₂, and hollow sphere silica, or may be a mixture of at least two materials selected from among TiO₂, ZnO, Al₂O₃, SiO₂, and hollow sphere silica.

The first light control part CCP1, the second light control part CCP2, and the third light control part CCP3 each may include base resins BR1, BR2, and BR3 in which the quantum dots QD1 and QD2 and the scatterer SP are dispersed. In one or more embodiments, the first light control part CCP1 may include the first quantum dot QD1 and the scatterer SP dispersed in a first base resin BR1, the second light control part CCP2 may include the second quantum dot QD2 and the scatterer SP dispersed in a second base resin BR2, and the third light control part CCP3 may include the scatterer SP dispersed in a third base resin BR3.

The base resins BR1, BR2, and BR3 are media in which the quantum dots QD1 and QD2 and the scatterer SP are dispersed, and may be formed of one or more suitable resin compositions, which may be generally referred to as a binder. For example, the base resins BR1, BR2, and BR3 may be acrylic-based resins, urethane-based resins, silicone-based resins, epoxy-based resins, and/or the like. The base resins BR1, BR2, and BR3 may be transparent resins. In one or more embodiments, the first base resin BR1, the second base resin BR2, and the third base resin BR3 may be the same as or different from each other.

The light control layer CCL may include a barrier layer BFL1. The barrier layer BFL1 may serve to prevent or reduce the penetration of moisture and/or oxygen (hereinafter, referred to as ‘moisture/oxygen’). The barrier layer BFL1 may block the light control parts CCP1, CCP2 and CCP3 from being exposed to moisture/oxygen. In one or more embodiments, the barrier layer BFL1 may cover the light control parts CCP1, CCP2, and CCP3. In some embodiments, the barrier layer BFL2 may be provided between the light control parts CCP1, CCP2, and CCP3 and the filters CF1, CF2, and CF3.

The barrier layers BFL1 and BFL2 may include at least one inorganic layer. For example, the barrier layers BFL1 and BFL2 may include an inorganic material. For example, the barrier layers BFL1 and BFL2 may include a silicon nitride, an aluminum nitride, a zirconium nitride, a titanium nitride, a hafnium nitride, a tantalum nitride, a silicon oxide, an aluminum oxide, a titanium oxide, a tin oxide, a cerium oxide, a silicon oxynitride, a metal thin film which secures a transmittance, and/or the like. In one or more embodiments, the barrier layers BFL1 and BFL2 may further include an organic film. The barrier layers BFL1 and BFL2 may be formed of a single layer or a plurality of layers.

In the display device DD-a of one or more embodiments, the color filter layer CFL may be arranged on the light control layer CCL. For example, the color filter layer CFL may be directly arranged on the light control layer CCL. In this case, the barrier layer BFL2 may not be provided.

The color filter layer CFL may include filters CF1, CF2, and CF3. Each of the first to third filters CF1, CF2, and CF3 can be arranged to correspond to a red light-emitting region PXA-R, a green light-emitting region PXA-G, and a blue light-emitting region PXA-B, respectively.

The color filter layer CFL may include a first filter CF1 configured to transmit the second color light, a second filter CF2 configured to transmit the third color light, and a third filter CF3 configured to transmit the first color light. For example, the first filter CF1 may be a red filter, the second filter CF2 may be a green filter, and the third filter CF3 may be a blue filter. The filters CF1, CF2, and CF3 each may include a polymeric photosensitive resin and a pigment or dye. The first filter CF1 may include a red pigment or dye, the second filter CF2 may include a green pigment or dye, and the third filter CF3 may include a blue pigment or dye.

In one or more embodiments, the embodiment of the disclosure is not limited thereto, and the third filter CF3 may not include (e.g., may exclude) a pigment or dye. The third filter CF3 may include a polymeric photosensitive resin and may not include a (e.g., may exclude any) pigment or dye. The third filter CF3 may be transparent. The third filter CF3 may be formed of a transparent photosensitive resin.

Furthermore, in one or more embodiments, the first filter CF1 and the second filter CF2 may be a yellow filter. The first filter CF1 and the second filter CF2 may not be separated but be provided as one filter.

Although not illustrated, the color filter layer CFL may further include a light shielding part. The light shielding part may be a black matrix. The light shielding part may include an organic light shielding material or an inorganic light shielding material containing a black pigment or dye. The light shielding part may prevent or reduce light leakage, and may separate boundaries between the adjacent filters CF1, CF2, and CF3.

A base substrate BL may be arranged on the color filter layer CFL. The base substrate BL may be a member which provides a base surface in which the color filter layer CFL, the light control layer CCL, and/or the like are arranged. The base substrate BL may be a glass substrate, a metal substrate, a plastic substrate, and/or the like. However, the embodiment of the disclosure is not limited thereto, and the base substrate BL may be an inorganic layer, an organic layer, or a composite material layer. In some embodiments, unlike the configuration illustrated, in one or more embodiments, the base substrate BL may not be provided.

FIG. 10 is a cross-sectional view illustrating a portion of a display device according to one or more embodiments. In the display device DD-TD of one or more embodiments, the light emitting element ED-BT may include a plurality of light emitting structures OL-B1, OL-B2, and OL-B3. The light emitting element ED-BT may include a first electrode EL1 and a second electrode EL2 which face each other, and the plurality of light emitting structures OL-B1, OL-B2, and OL-B3 sequentially stacked in the thickness direction between the first electrode EL1 and the second electrode EL2. The light emitting structures OL-B1, OL-B2, and OL-B3 each may include an emission layer EML (FIG. 9) and a hole transport region HTR and an electron transport region ETR arranged with the emission layer EML (FIG. 9) located therebetween.

For example, the light emitting element ED-BT included in the display device DD-TD of one or more embodiments may be a light emitting element having a tandem structure and including a plurality of emission layers.

In one or more embodiments illustrated in FIG. 10, all light beams respectively emitted from the light emitting structures OL-B1, OL-B2, and OL-B3 may be blue light. However, the embodiment of the disclosure is not limited thereto, and the light beams respectively emitted from the light emitting structures OL-B1, OL-B2, and OL-B3 may have wavelength ranges different from each other. For example, the light emitting element ED-BT including the plurality of light emitting structures OL-B1, OL-B2, and OL-B3 which emit light beams having wavelength ranges different from each other may be to emit white light.

Charge generation layers CGL1 and CGL2 may be respectively arranged between two of the neighboring light emitting structures OL-B1, OL-B2, and OL-B3. The charge generation layers CGL1 and CGL2 may include a p-type (kind) charge generation layer and/or an n-type (kind) charge generation layer.

At least one of the light emitting structures OL-B1, OL-B2, and OL-B3 included in the display device DD-TD of one or more embodiments may include the nitrogen-containing compound of one or more embodiments described herein. For example, at least one of the plurality of emission layers included in the light emitting element ED-BT may include the nitrogen-containing compound of one or more embodiments. The light emitting element ED-BT according to one or more embodiments may exhibit high efficiency and long life characteristics. The display device DD-TD of one or more embodiments includes the light emitting element ED-BT containing a nitrogen-containing compound according to one or more embodiments, and may thus exhibit excellent or suitable display quality.

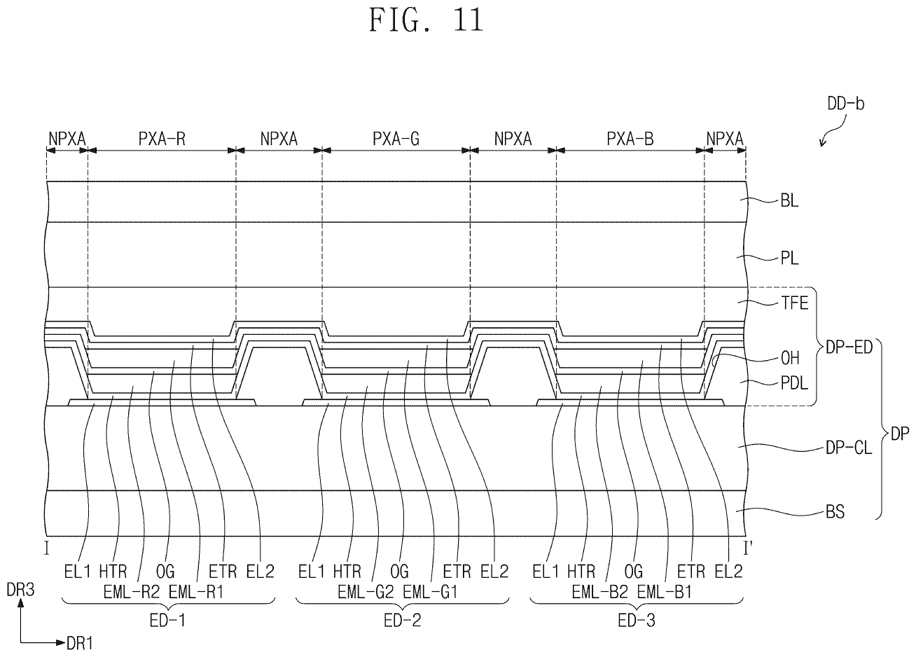

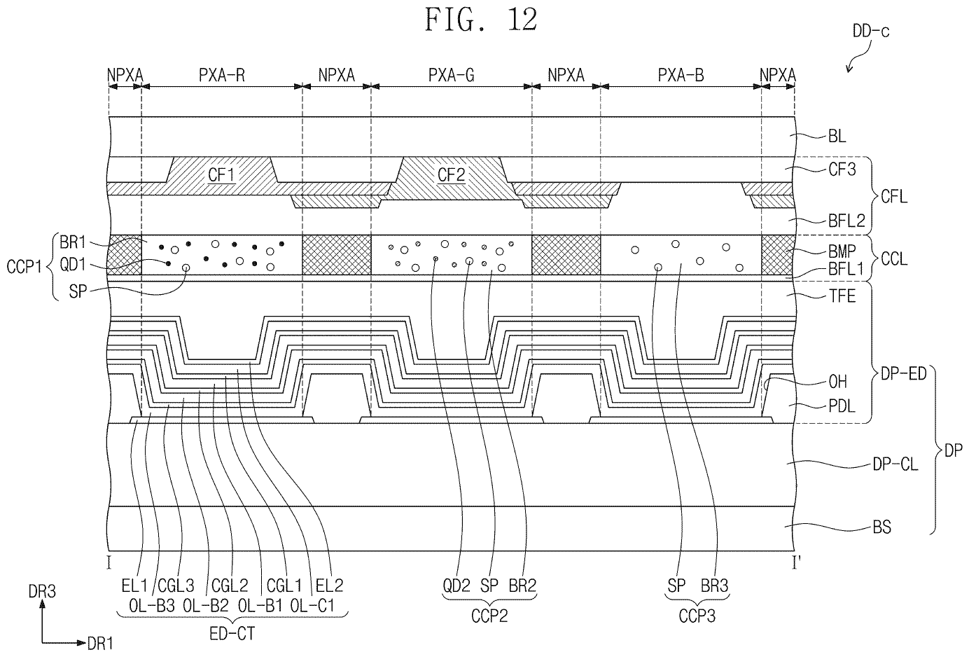

FIG. 11 is a cross-sectional view showing a display device according to one or more embodiments of the disclosure. FIG. 12 is a cross-sectional view showing a display device according to one or more embodiments of the disclosure.

Referring to FIG. 11, the display device DD-b according to one or more embodiments may include light emitting elements ED-1, ED-2, and ED-3 in which two emission layers are stacked. Compared with the display device DD of one or more embodiments illustrated in FIG. 4, one or more embodiments illustrated in FIG. 11 has a difference in that the first to third light emitting elements ED-1, ED-2, and ED-3 each include two emission layers stacked in the thickness direction. In each of the first to third light emitting elements ED-1, ED-2, and ED-3, the two emission layers may be to emit light in substantially the same wavelength region.

The first light emitting element ED-1 may include a first red emission layer EML-R1 and a second red emission layer EML-R2. The second light emitting element ED-2 may include a first green emission layer EML-G1 and a second green emission layer EML-G2. In some embodiments, the third light emitting element ED-3 may include a first blue emission layer EML-B1 and a second blue emission layer EML-B2. An emission auxiliary part OG may be arranged between the first red emission layer EML-R1 and the second red emission layer EML-R2, between the first green emission layer EML-G1 and the second green emission layer EML-G2, and between the first blue emission layer EML-B1 and the second blue emission layer EML-B2.

The emission auxiliary part OG may include a single layer or a multilayer. The emission auxiliary part OG may include a charge generation layer. For example, the emission auxiliary part OG may include an electron transport region, a charge generation layer, and a hole transport region that are sequentially stacked. The emission auxiliary part OG may be provided as a common layer in the whole of the first to third light emitting elements ED-1, ED-2, and ED-3. However, the embodiment of the disclosure is not limited thereto, and the emission auxiliary part OG may be provided by being patterned within the openings OH defined in the pixel defining film PDL.

The first red emission layer EML-R1, the first green emission layer EML-G1, and the first blue emission layer EML-B1 may be arranged between the emission auxiliary part OG and the electron transport region ETR. The second red emission layer EML-R2, the second green emission layer EML-G2, and the second blue emission layer EML-B2 may be arranged between the hole transport region HTR and the emission auxiliary part OG.

For example, the first light emitting element ED-1 may include the first electrode EL1, the hole transport region HTR, the second red emission layer EML-R2, the emission auxiliary part OG, the first red emission layer EML-R1, the electron transport region ETR, and the second electrode EL2 that are sequentially stacked. The second light emitting element ED-2 may include the first electrode EL1, the hole transport region HTR, the second green emission layer EML-G2, the emission auxiliary part OG, the first green emission layer EML-G1, the electron transport region ETR, and the second electrode EL2 that are sequentially stacked. The third light emitting element ED-3 may include the first electrode EL1, the hole transport region HTR, the second blue emission layer EML-B2, the emission auxiliary part OG, the first blue emission layer EML-B1, the electron transport region ETR, and the second electrode EL2 that are sequentially stacked.

In one or more embodiments, an optical auxiliary layer PL may be arranged on the display device layer DP-ED. The optical auxiliary layer PL may include a polarizing layer. The optical auxiliary layer PL may be arranged on the display panel DP and control reflected light in the display panel DP due to external light. Unlike the configuration illustrated, the optical auxiliary layer PL in the display device according to one or more embodiments may not be provided.

At least one emission layer included in a display device DD-b according to one or more embodiments shown in FIG. 11 may include the nitrogen-containing compound according to one or more embodiments described herein. For example, at least one of the first blue emission layer EML-B1 or the second blue emission layer EML-B2 may include the nitrogen-containing compound according to one or more embodiments.

Unlike FIGS. 10 and 11, FIG. 12 illustrates that a display device DD-c includes four light emitting structures OL-B1, OL-B2, OL-B3, and OL-C1. A light emitting element ED-CT may include a first electrode EL1 and a second electrode EL2 which face each other, and first to fourth light emitting structures OL-B1, OL-B2, OL-B3, and OL-C1 that are sequentially stacked in the thickness direction between the first electrode EL1 and the second electrode EL2. Charge generation layers CGL1, CGL2, and CGL3 may be arranged between the first to fourth light emitting structures OL-B1, OL-B2, OL-B3, and OL-C1. Among the four light emitting structures, the first to third light emitting structures OL-B1, OL-B2, and OL-B3 may be to emit blue light, and the fourth light emitting structure OL-C1 may be to emit green light. However, the embodiment of the disclosure is not limited thereto, and the first to fourth light emitting structures OL-B1, OL-B2, OL-B3, and OL-C1 may be to emit light beams in different wavelength regions.

The charge generation layers CGL1, CGL2, and CGL3 arranged between adjacent light emitting structures OL-C1, OL-B1, OL-B2, and OL-B3 may include a p-type (kind) charge generation layer and/or an n-type (kind) charge generation layer.

At least one of the light-emitting structures OL-B1, OL-B2, OL-B3, and OL-C1 included in the display device DD-c of one or more embodiments may include the nitrogen-containing compound of one or more embodiments described herein. For example, at least one of the first to third light-emitting structures OL-B1, OL-B2, and OL-B3 of one or more embodiments may include the nitrogen-containing compound of one or more embodiments described herein. Accordingly, the light emitting element ED-CT may exhibit characteristics of high efficiency and long life.

In one or more embodiments, the electronic device may include a display device including a plurality of light emitting elements, and a control part which controls the display device. The electronic device of one or more embodiments may be a device that is activated according to an electrical signal. The electronic device may include display devices of one or more suitable embodiments. The electronic devices may include large-sized electronic devices such as televisions, monitors, or outdoor billboards and small- and medium-sized electronic devices such as personal computers; laptop computers; personal digital terminals such as smartphones and tablets; car navigation units; game consoles; smart watches; or cameras. In some embodiments, these devices are merely provided as embodiments, and the display device according to one or more embodiments may also be employed in other electronic devices as long as not departing from the disclosure.

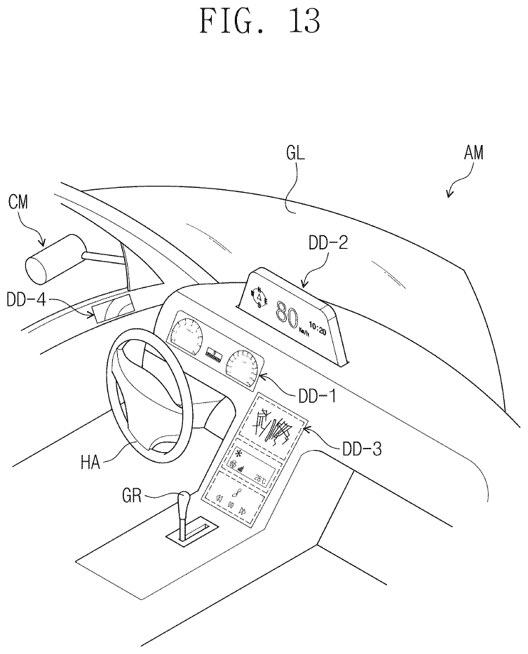

FIG. 13 is a view illustrating a vehicle AM in which first to fourth display devices DD-1, DD-2, DD-3, and DD-4 are arranged. At least one among the first to fourth display devices DD-1, DD-2, DD-3, and DD-4 may include the same configuration as the display devices DD, DD-TD, DD-a, DD-b, and DD-c as described with reference to FIGS. 3, and 4, and 9 to 12.

FIG. 13 illustrates a vehicle AM, but this is an example, and the first to fourth display devices DD-1, DD-2, DD-3, and DD-4 may be arranged in another transportation refers to such as bicycles, motorcycles, trains, ships, and airplanes. In some embodiments, at least one among the first to fourth display devices DD-1, DD-2, DD-3, and DD-4 including the same configuration as the display devices DD, DD-TD, DD-a, DD-b, and DD-c of one or more embodiments may be employed in a personal computer, a laptop computer, a personal digital terminal, a game console, a portable electronic device, a television, a monitor, an outdoor billboard, and/or the like. In some embodiments, these are merely provided as embodiments, and thus may be employed in other electronic devices unless departing from the disclosure.

At least one among the first to fourth display devices DD-1, DD-2, DD-3, and DD-4 may include the light emitting element ED of one or more embodiments as described with reference to FIGS. 5 to 8. The light emitting element ED of one or more embodiments may include the nitrogen compound of one or more embodiments. At least one of the first to fourth display devices DD-1, DD-2, DD-3, and DD-4 may include the light emitting element ED including the nitrogen compound of one or more embodiments, so that the display lifespan may be improved.

Referring to FIG. 13, the vehicle AM may include a steering wheel HA and a gear GR for driving the vehicle AM. In some embodiments, the vehicle AM may include a front window GL arranged so as to face the driver.

The first display device DD-1 may be arranged in a first region overlapping the steering wheel HA. For example, the first display device DD-1 may be a digital cluster which displays first information of the vehicle AM. The first information may include a first scale which indicates a driving speed of the vehicle AM, a second scale which indicates an engine speed (that is, revolutions per minute (RPM)), an image which indicates a fuel state, and/or the like. A first scale and a second scale may be indicated as a digital image.

The second display device DD-2 may be arranged in a second region opposite to (e.g., facing) the driver's seat and overlapping the front window GL. The driver's seat may be a seat in which the steering wheel HA is arranged. For example, the second display device DD-2 may be a head up display (HUD) which displays second information of the vehicle AM. The second display device DD-2 may be optically transparent. The second information may include digital numbers which indicate a driving speed, and may further include information such as the current time. Unlike the configuration illustrated, the second information of the second display device DD-2 may be projected to the front window GL to be displayed.

The third display device DD-3 may be arranged in a third region adjacent to the gear GR. For example, the third display device DD-3 may be arranged between the driver's seat and the passenger seat and may be a center information display (CID) for a vehicle for displaying third information. The passenger seat may be a seat spaced and/or apart (e.g., spaced apart or separated) from the driver's seat with the gear GR arranged therebetween. The third information may include information about traffic (e.g., navigation information), playing music or radio or a video (or an image), temperatures inside the vehicle AM, and/or the like.

The fourth display device DD-4 may be spaced and/or apart (e.g., spaced apart or separated) from the steering wheel HA and the gear GR, and may be arranged in a fourth region adjacent to the side of the vehicle AM. For example, the fourth display device DD-4 may be a digital side-view mirror which displays fourth information. The fourth display device DD-4 may display an image outside the vehicle AM taken by a camera module CM arranged outside the vehicle AM. The fourth information may include an image outside the vehicle AM.

The herein-described first to fourth information may be examples, and the first to fourth display devices DD-1, DD-2, DD-3, and DD-4 may further display information about the inside and outside of the vehicle AM. The first to fourth information may include different information. However, the embodiment of the disclosure is not limited thereto, and a part of the first to fourth information may include the same information as one another.

Hereinafter, with reference to Examples and Comparative Examples, a nitrogen-containing compound according to one or more embodiments of the disclosure and a light emitting element according to one or more embodiments will be specifically described. In some embodiments, Examples are shown only for the understanding of the disclosure, and the scope of the disclosure is not limited thereto.

### Examples

### (1) Synthesis of Compound ET-1

Nitrogen-containing compound ET-1 according to one or more embodiments may be synthesized by, for example, Reaction Formula 1.

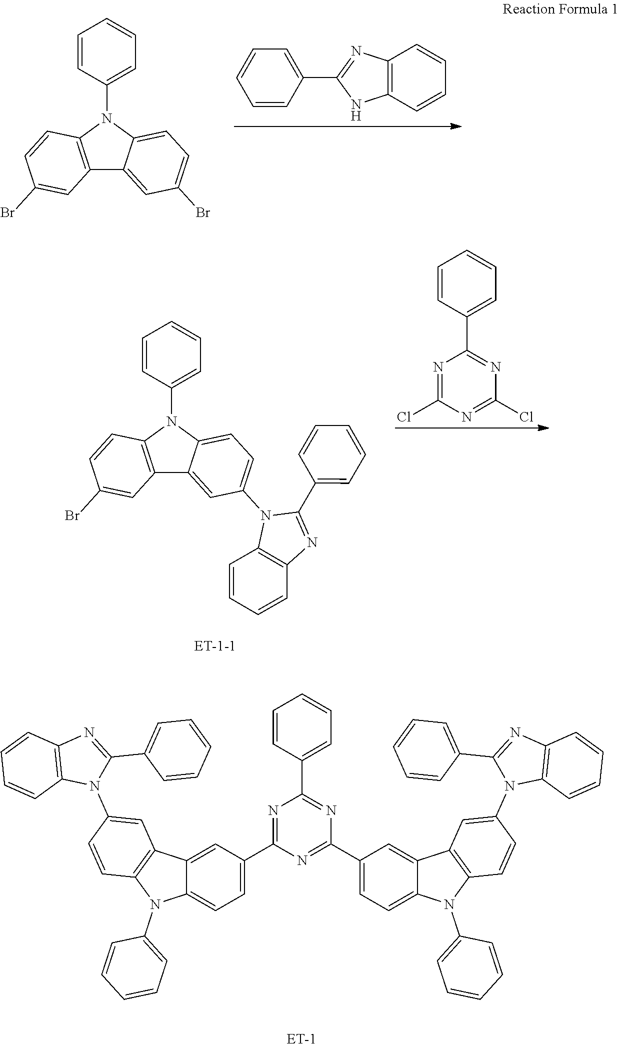 [SMILES: Brc1ccc2c(c1)c1cc(Br)ccc1n2-c1ccccc1 ; c1ccc(-c2nc3ccccc3[nH]2)cc1 ; Brc1ccc2c(c1)c1cc(-n3c(-c4ccccc4)nc4ccccc43)ccc1n2-c1ccccc1 ; Clc1nc(Cl)nc(-c2ccccc2)n1 ; c1ccc(-c2nc(-c3ccc4c(c3)c3cc(-n5c(-c6ccccc6)nc6ccccc65)ccc3n4-c3ccccc3)nc(-c3ccc4c(c3)c3cc(-n5c(-c6ccccc6)nc6ccccc65)ccc3n4-c3ccccc3)n2)cc1]

### 1) Synthesis of Intermediate ET-1-1

3,6-dibromo-9-phenyl-9H-carbazole, 2-phenyl-1H-benzo[d]imidazole, sodium tert-butoxide, tri-tert-butylphosphine, and Pd₂(dba)₃ were dissolved in toluene and then subjected to a reaction at 120° C. for 12 hours to synthesize Intermediate ET-1-1.

Intermediate ET-1-1 was characterized through LC/MS.

Intermediate ET-1-1 LCMS detected: C₃₁H₂₀BrN₃ M+1: 515.08

### 2) Synthesis of Compound ET-1

5.0 g of Intermediate ET-1-1, 2.19 g of 2,4-dichloro-6-phenyl-1,3,5-triazine, 1.40 g of sodium tert-butoxide, 0.16 g of tri-tert-butylphosphine, and 0.36 g of Pd₂(dba)₃ were dissolved in toluene and then subjected to a reaction at 120° C. for 12 hours. After the reaction was completed, the resulting product was extracted with methylene chloride (MC) and subjected to column purification to synthesize 5.77 g (yield: 58%) of Compound ET-1. Compound ET-1 was characterized through LC/MS. Compound ET-1 LCMS detected: C₇₁H₄₅N₉ (MS/FAB): 1024.38

### 2. Preparation and Evaluation of Light Emitting Element

Light emitting elements of one or more embodiments containing a nitrogen-containing compound of one or more embodiments in an emission layer were prepared using the following method. A light emitting element of Example 1 was prepared using the nitrogen-containing compound ET-1, which is the Example Compound described herein, as a host material of the emission layer. Comparative Examples 1 to 4 correspond to light emitting elements prepared using Comparative Example Compounds C1 to C4 each as a host material of the emission layer.

### Example Compound

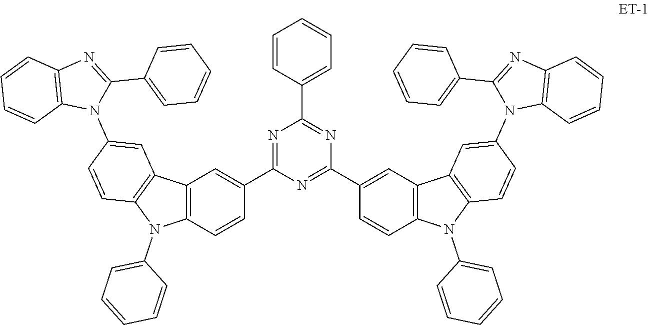 [SMILES: c1ccc(-c2nc(-c3ccc4c(c3)c3cc(-n5c(-c6ccccc6)nc6ccccc65)ccc3n4-c3ccccc3)nc(-c3ccc4c(c3)c3cc(-n5c(-c6ccccc6)nc6ccccc65)ccc3n4-c3ccccc3)n2)cc1]

### Comparative Example Compounds

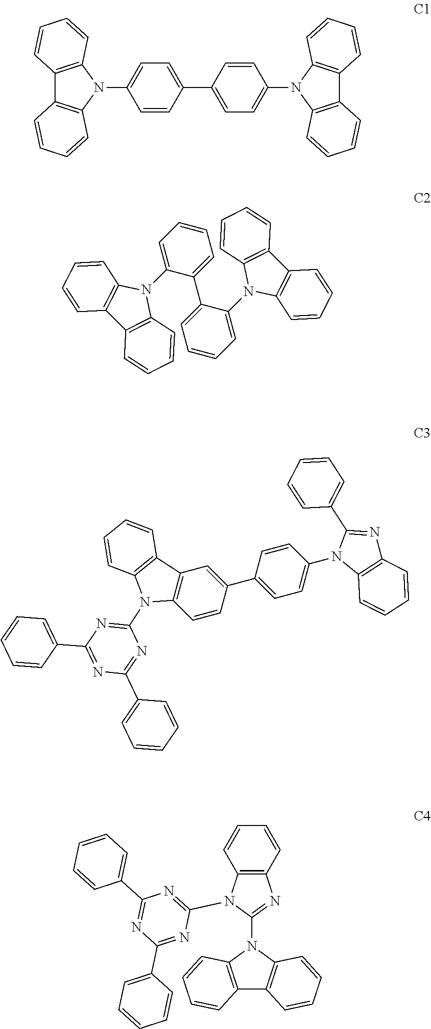 [SMILES: c1ccc2c(c1)c1ccccc1n2-c1ccc(-c2ccc(-n3c4ccccc4c4ccccc43)cc2)cc1 ; c1ccc(-n2c3ccccc3c3ccccc32)c(-c2ccccc2-n2c3ccccc3c3ccccc32)c1 ; c1ccc(-c2nc(-c3ccccc3)nc(-n3c4ccccc4c4cc(-c5ccc(-n6c(-c7ccccc7)nc7ccccc76)cc5)ccc43)n2)cc1 ; c1ccc(-c2nc(-c3ccccc3)nc(-n3c(-n4c5ccccc5c5ccccc54)nc4ccccc43)n2)cc1]

### (1) Preparation of Light Emitting Element

Light emitting elements of one or more embodiments containing a nitrogen-containing compound of one or more embodiments in an emission layer were prepared using the following method. A light emitting element of Example 1 was prepared using Compound ET-1 as a host material of the emission layer. Light emitting elements of Comparative Examples 1 to 4 were prepared using Comparative Example Compounds C1 to C4 as a host material of the emission layer. Comparative Example Compound C2 used in the light emitting element of Comparative Example 2 is the same material as Compound HT6.

As for light emitting elements of Examples and Comparative Examples, as a first electrode, a glass substrate (Corning) having an ITO electrode formed thereon was cut to a size of 50 millimeter (mm)×50 mm×0.5 mm, subjected to ultrasonic cleaning using isopropyl alcohol and pure water each for 10 minutes and ultraviolet irradiation for 10 minutes, and then exposed to ozone for cleaning to be mounted on a vacuum deposition apparatus.

On the first electrode, a hole injection layer having a thickness of 40 angstrom (Å) was formed through the deposition of m-MTDATA, and then on an upper portion of the hole injection layer, a hole transport layer having a thickness of 10 Å was formed through the deposition of NPB.

On the hole transport layer, an emission layer having a thickness of 300 Å was formed through the co-deposition of a host and dopant TA-1. In this case, the host was a mixture of a first compound and a second compound in a weight ratio of 5:5, using Example compound or Comparative Example Compound as the first compound, and using Compound HT6 as the second compound.

Then, on an upper portion of the emission layer, an electron transport layer having a thickness of 300 Å was formed through the deposition of Compound ET12. On an upper portion of the electron transport layer, a second electrode of Al was formed through the deposition of Aluminum (Al) with a thickness of 1200 Å to prepare a light emitting element. Each layer was formed through vacuum deposition.

The compounds used in the preparation of the light emitting elements of Examples and Comparative Examples are disclosed.

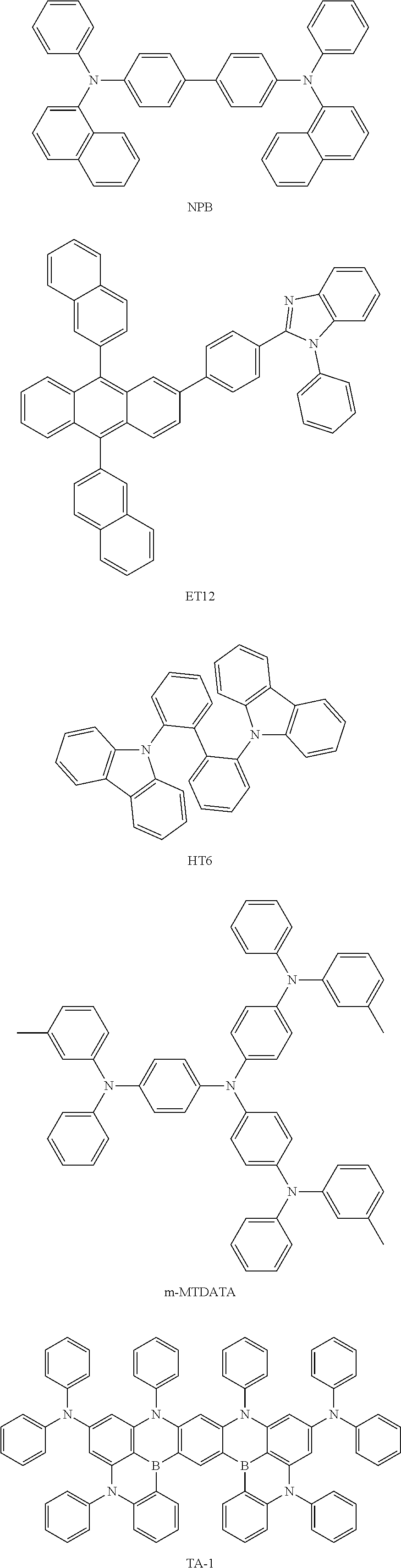

### (2) Property Evaluation of Light Emitting Element

Element efficiency and element life of the light emitting elements prepared using Example Compound ET-1, and Comparative Example Compounds C1 to C4 described herein were evaluated. Table 1 shows results of evaluation on light emitting elements for Example 1 and Comparative Examples 1 to 4. In the light emitting elements of Examples and Comparative Examples, as for the characteristics of the light emitting elements, luminous efficacy (candela per ampere (cd/A)) was measured at a current density of 10 milliampere per square centimeter (mA/cm²) using a source meter (Keithley Instrument, 2400 series). Life (T₉₀) was determined by measuring the time from an initial value to 90% luminance degradation under substantially continuous operation at a current density of 10 mA/cm².

TABLE 1

| | | Efficiency | Life |
|---|---|---|---|
| Item | Emission layer host | (cd/A) | (T₉₀, hour) |
| Example 1 | Compound ET-1 | 26.8 | 59.7 |
| Comparative | Comparative Example | 16 | 8 |
| Example 1 | Compound C1 | | |
| Comparative | Comparative Example | 20 | 11 |
| Example 2 | Compound C2 | | |
| Comparative | Comparative Example | 24.6 | 38.9 |
| Example 3 | Compound C3 | | |
| Comparative | Comparative Example | 23.8 | 29.4 |
| Example 4 | Compound C4 | | |

Referring to the results in Table 1, when the nitrogen-containing compound according to one or more embodiments of the disclosure is used as a host material for an emission layer of a light emitting element, high efficiency and long life may be achieved. For example, it is determined that Example 1 exhibits higher efficiency and longer life than Comparative Examples 1 to 4.

The nitrogen-containing compound according to one or more embodiments of the disclosure has a structure in which two heterocycle substituents are connected to a core of a six-membered ring containing at least one nitrogen atom as a ring-forming atom through a linker of a heteroarylene group having 6 or more ring-forming carbon atoms, and may thus exhibit excellent or suitable electron transport properties. Accordingly, a light emitting element includes the nitrogen-containing compound of one or more embodiments in an emission layer, and may thus exhibit both (e.g., simultaneously) high efficiency and long life characteristics.

Comparative Example Compounds C1 and C2 included in the light emitting elements of Comparative Examples 1 and 2 correspond to compounds that do not include a core of a six-membered ring containing at least one nitrogen atom as a ring-forming atom, compared to Example Compounds. Accordingly, it is determined that Comparative Example Compounds C1 and C2 have reduced electron transport characteristics compared to Example Compounds, and have reduced luminous efficiency and life characteristics when applied to a light emitting element, compared to Examples.

Comparative Example Compounds C3 and C4 used in the light emitting elements of Comparative Examples 3 and 4 correspond to a structure in which one heterocycle substituent is connected through a linker, or one heterocycle substituent is directly connected to the core, compared to Example Compounds. Accordingly, it is determined that the light emitting elements of Comparative Examples 3 and 4 exhibit reduced efficiency and life compared to the light emitting elements of Examples.

For example, the experimental results presented above demonstrate that the structural features of the nitrogen-containing compounds disclosed herein—specifically, the incorporation of a six-membered nitrogen-containing core and dual heterocyclic substituents linked via heteroarylene groups—contribute directly to enhanced charge transport and exciton confinement within the emission layer. These molecular characteristics result in a synergistic improvement in both luminous efficiency and operational lifetime of the light emitting elements. The comparative data further validate that such improvements are not incidental, but rather attributable to the specific molecular architecture of the disclosed compounds. Accordingly, the present disclosure provides an enhanced solution for achieving high-performance organic light emitting elements and display devices, particularly in applications requiring both energy efficiency and long-term reliability.

The light emitting element of one or more embodiments includes the nitrogen-containing compound of one or more embodiments as a host for the emission layer of the light emitting element, and may thus achieve high element efficiency and improved life characteristics.

A light emitting element of one or more embodiments may exhibit improved element characteristics of high efficiency and long service life.

A nitrogen-containing compound of one or more embodiments is included in an emission layer of a light emitting element, and may thus contribute to high efficiency and long service life of the light emitting element.

A display device of one or more embodiments may exhibit high display quality.

The display device, the electronic device, a manufacturing device of the same, and/or any other relevant devices or components according to embodiments of the present invention described herein may be implemented utilizing any suitable hardware, firmware (e.g., an application-specific integrated circuit), software, or a combination of software, firmware, and hardware. For example, the various components of the device may be formed on one integrated circuit (IC) chip or on separate IC chips. Further, the various components of the device may be implemented on a flexible printed circuit film, a tape carrier package (TCP), a printed circuit board (PCB), or formed on one substrate. Further, the various components of the device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the present disclosure.

In the above, description has been made with reference to one or more embodiments of the disclosure, but those skilled or of ordinary skill in the art may understand that one or more suitable modifications and changes may be made to the disclosure insofar as such modifications and changes do not depart from the spirit and technical scope of the disclosure set forth in the claims to be described later. Also, descriptions of features or aspects within each embodiment should typically be considered as available for other similar features or aspects in other embodiments.

Therefore, the technical scope of the disclosure is not to be limited to the contents stated in the detailed description of the specification, but should be determined by the claims and equivalents thereof.

What is claimed is:

1. A light emitting element comprising:
a first electrode;
a second electrode on the first electrode; and
an emission layer between the first electrode and the second electrode and comprising a first compound,
wherein,
the first compound is represented by Formula 1: 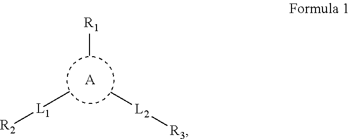
in Formula 1,
ring A is a substituted or unsubstituted heterocycle having 3 ring-forming carbon atoms, three ring-forming heteroatoms selected from among a nitrogen atom, an oxygen atom, and a sulfur atom, and at least one ring-forming nitrogen atom,
L₁ and L₂ are each independently a substituted or unsubstituted heteroarylene group having 6 to 30 ring-forming carbon atoms, having at least one ring-forming nitrogen atom,
R₁ is a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms,
R₂ and R₃ are each independently represented by Formula S-1 or Formula S-2: 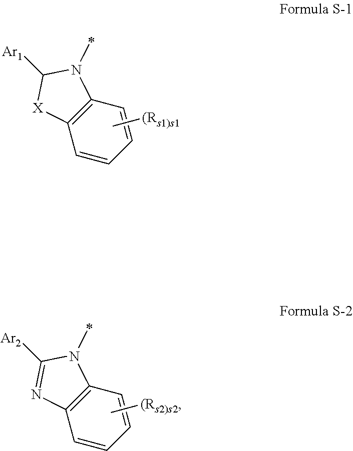
and
wherein,
in Formulas S-1 and S-2,
X is NRₛ₃, O, or S,
Ar₁ and Ar₂ are each independently a substituted or unsubstituted aryl group having 6 to 60 ring-forming carbon atoms, or a substituted or unsubstituted heteroaryl group having 2 to 60 ring-forming carbon atoms,
Rₛ₁ to Rₛ₃ are each independently a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms,
s1 and s2 are each independently an integer of 0 to 4, and [symbol]
is a site linked to Formula 1.

2. The light emitting element of claim 1, wherein the first compound is represented by Formula 2-1 or Formula 2-2: 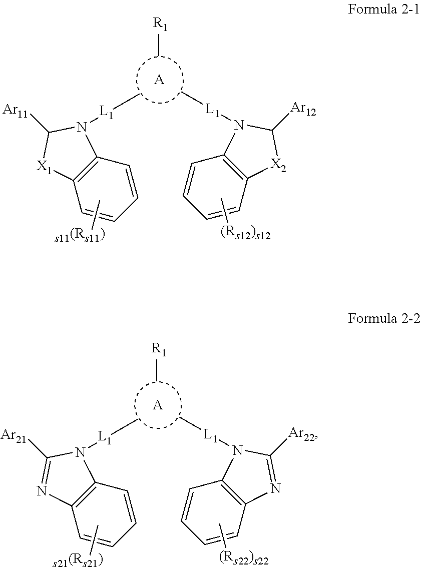
wherein,
in Formulas 2-1 and 2-2,
X₁ and X₂ are each independently O or S,
Ar₁₁, Ar₁₂, Ar₂₁, and Ar₂₂ are each independently a substituted or unsubstituted aryl group having 6 to 30 ring-forming carbon atoms,
Rₛ₁₁, Rₛ₁₂, Rₛ₂₁, and Rₛ₂₂ are each independently a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms,
s11, s12, s21, and s22 are each independently an integer of 0 to 4, and
ring A, L₁, L₂, and R₁ are as defined in Formula 1.

3. The light emitting element of claim 1, wherein the first compound is represented by Formula 3-1 or Formula 3-2: 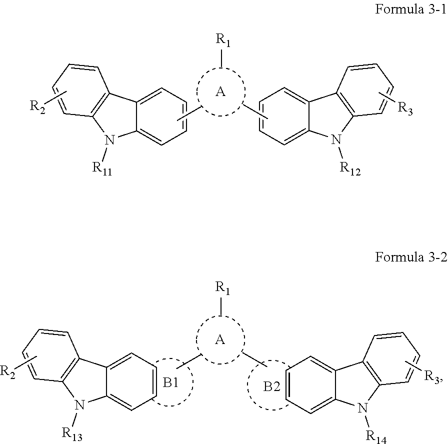
and
wherein,
in Formulas 3-1 and 3-2,
ring B1 and ring B2 are each independently a substituted or unsubstituted hydrocarbon ring having 5 to 30 ring-forming carbon atoms, or a substituted or unsubstituted heterocycle having 2 to 30 ring-forming carbon atoms,
R₁₁, R₁₂, R₁₃, and R₁₄ are each independently a substituted or unsubstituted aryl group having 6 to 30 ring-forming carbon atoms, and
ring A, R₁, R₂, and R₃ are as defined in Formula 1.

4. The light emitting element of claim 3, wherein the first compound is represented by Formula 3-1-1 or Formula 3-1-2: 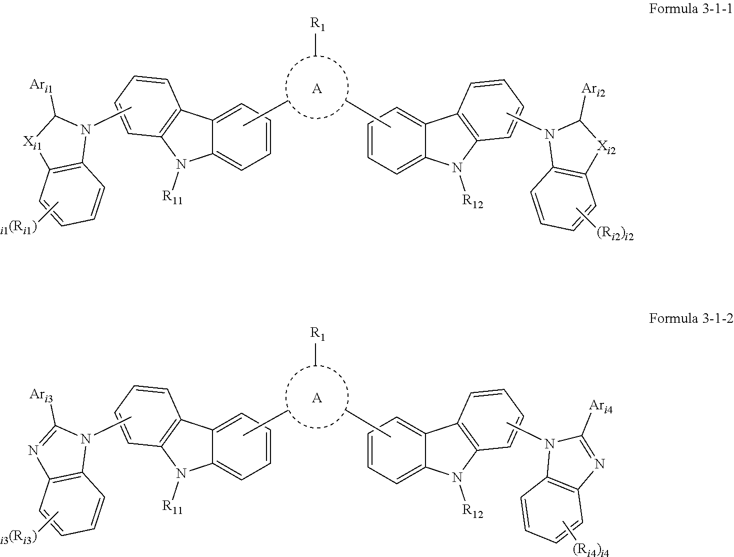
wherein,
in Formulas 3-1-1 and 3-1-2,
Xᵢ₁ and Xᵢ₂ are each independently O or S,
Arᵢ₁, Arᵢ₂, Arᵢ₁, and Arᵢ₂ are each independently a substituted or unsubstituted aryl group having 6 to 30 ring-forming carbon atoms,
Rᵢ₁, Rᵢ₂, Rᵢ₃, and Rᵢ₄ are each independently a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms,
i1, i2, i3, and i4 are each independently an integer of 0 to 4, and
ring A, R₁, R₁₁, and R₁₂ are as defined in Formulas 1 and 3-1.

5. The light emitting element of claim 3, wherein the first compound is represented by Formula 3-2-1 or Formula 3-2-2: 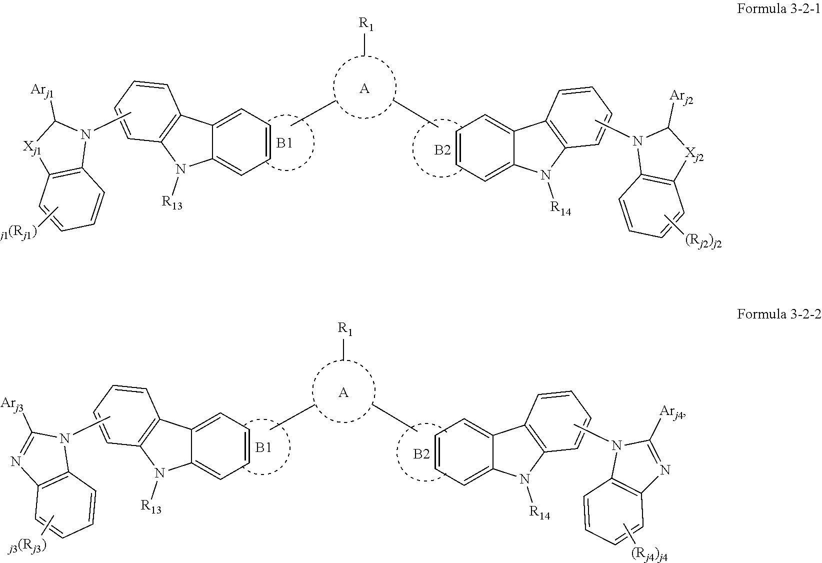
wherein,
in Formulas 3-2-1 and 3-2-2,
Xⱼ₁ and Xⱼ₂ are each independently O or S,
Arⱼ₁, Arⱼ₂, Arⱼ₁, and Arⱼ₂ are each independently a substituted or unsubstituted aryl group having 6 to 30 ring-forming carbon atoms,
Rⱼ₁, Rⱼ₂, Rⱼ₃, and Rⱼ₄ are each independently a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms,
j1, j2, j3, and j4 are each independently an integer of 0 to 4, and
ring A, ring B1, ring B2, R₁, R₁₃, and R₁₄ are as defined in Formulas 1 and 3-2.

6. The light emitting element of claim 1, wherein the first compound is represented by Formula 4 or Formula 5: 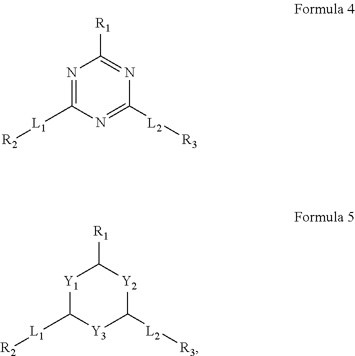
and
wherein,
in Formula 5,
at least one selected from among Y₁ to Y₃ is NR₄, and any remaining Y₁ to Y₃ is each independently O or S, and
R₄ is a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms, and
in Formulas 4 and 5,
L₁, L₂, and R₁ to R₃ are as defined in Formula 1.

7. The light emitting element of claim 1, wherein the first compound is represented by Formula 4-1 or Formula 4-2: 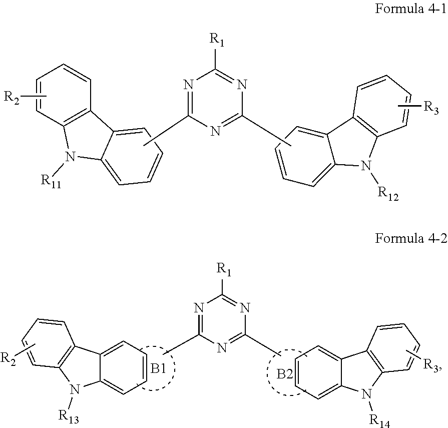
and
wherein,
in Formulas 4-1 and 4-2,
ring B1 and ring B2 are each independently a substituted or unsubstituted hydrocarbon ring having 5 to 30 ring-forming carbon atoms, or a substituted or unsubstituted heterocycle having 2 to 30 ring-forming carbon atoms,
R₁₁, R₁₂, R₁₃, and R₁₄ are each independently a substituted or unsubstituted aryl group having 6 to 30 ring-forming carbon atoms, and
ring A, R₁, R₂, and R₃ are as defined in Formula 1.

8. The light emitting element of claim 1, wherein the emission layer further comprises a second compound represented by Formula HT: 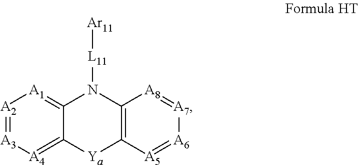
and
wherein,
in Formula HT,
A₁ to A₈ are each independently N or CR₅₁,
L₁₁ is a direct linkage, a substituted or unsubstituted arylene group having 6 to 30 ring-forming carbon atoms, or a substituted or unsubstituted heteroarylene group having 2 to 30 ring-forming carbon atoms,
Yₐ is a direct linkage, CR₅₂R₅₃, or SiR₅₄R₅₅,
Ar₁₁ is a substituted or unsubstituted aryl group having 6 to 30 ring-forming carbon atoms, or a substituted or unsubstituted heteroaryl group having 2 to 30 ring-forming carbon atoms, and
R₅₁ to R₅₅ are each independently a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 60 ring-forming carbon atoms, or a substituted or unsubstituted heteroaryl group having 2 to 60 ring-forming carbon atoms, or bonded to an adjacent group to form a ring.

9. The light emitting element of claim 1, wherein the emission layer comprises a host and a dopant,
the host comprising the first compound.

10. The light emitting element of claim 9, wherein the emission layer is configured to emit thermally activated delayed fluorescence.

11. The light emitting element of claim 1, wherein the first compound is represented by any one selected from among the compounds of Compound Group 1:

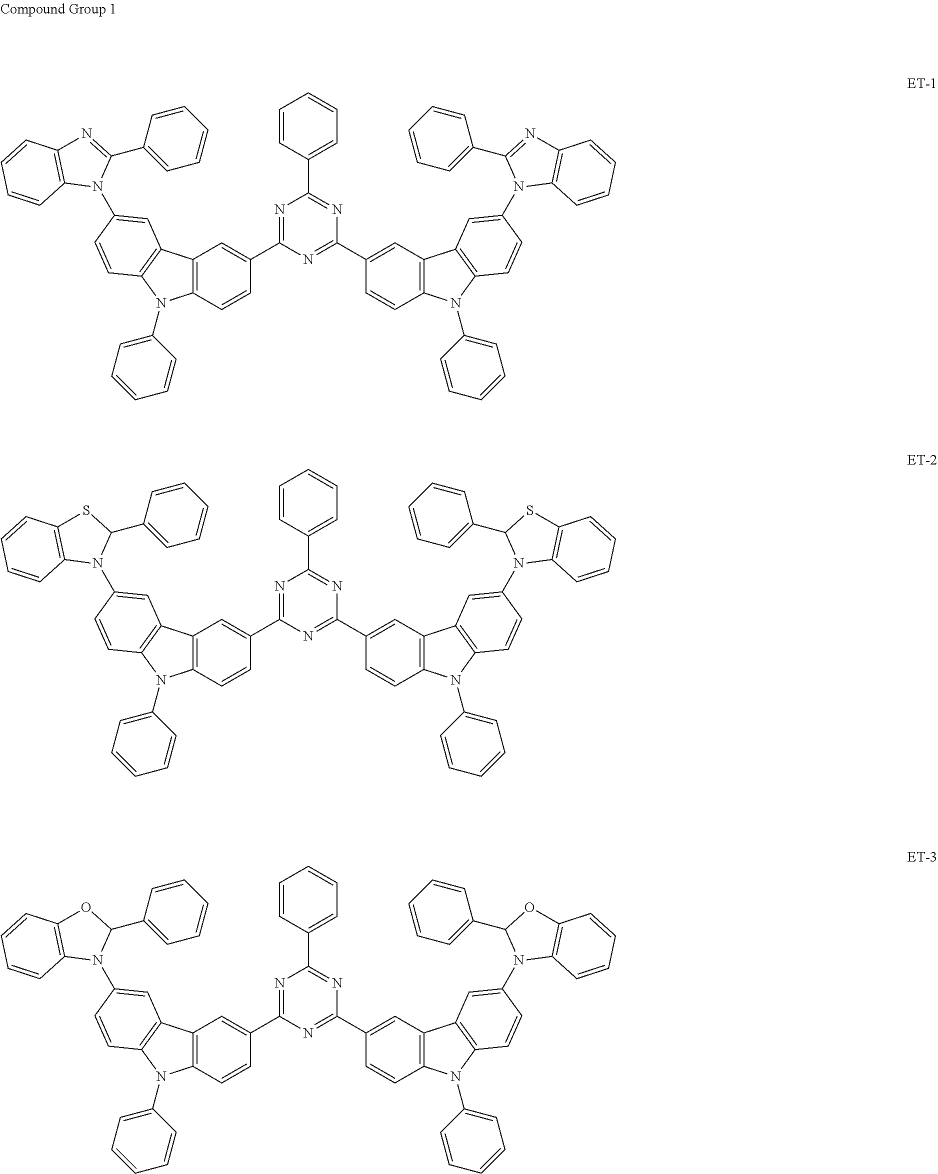 [SMILES: c1ccc(-c2nc(-c3ccc4c(c3)c3cc(-n5c(-c6ccccc6)nc6ccccc65)ccc3n4-c3ccccc3)nc(-c3ccc4c(c3)c3cc(-n5c(-c6ccccc6)nc6ccccc65)ccc3n4-c3ccccc3)n2)cc1 ; c1ccc(-c2nc(-c3ccc4c(c3)c3cc(N5c6ccccc6SC5c5ccccc5)ccc3n4-c3ccccc3)nc(-c3ccc4c(c3)c3cc(N5c6ccccc6SC5c5ccccc5)ccc3n4-c3ccccc3)n2)cc1 ; c1ccc(-c2nc(-c3ccc4c(c3)c3cc(N5c6ccccc6OC5c5ccccc5)ccc3n4-c3ccccc3)nc(-c3ccc4c(c3)c3cc(N5c6ccccc6OC5c5ccccc5)ccc3n4-c3ccccc3)n2)cc1]

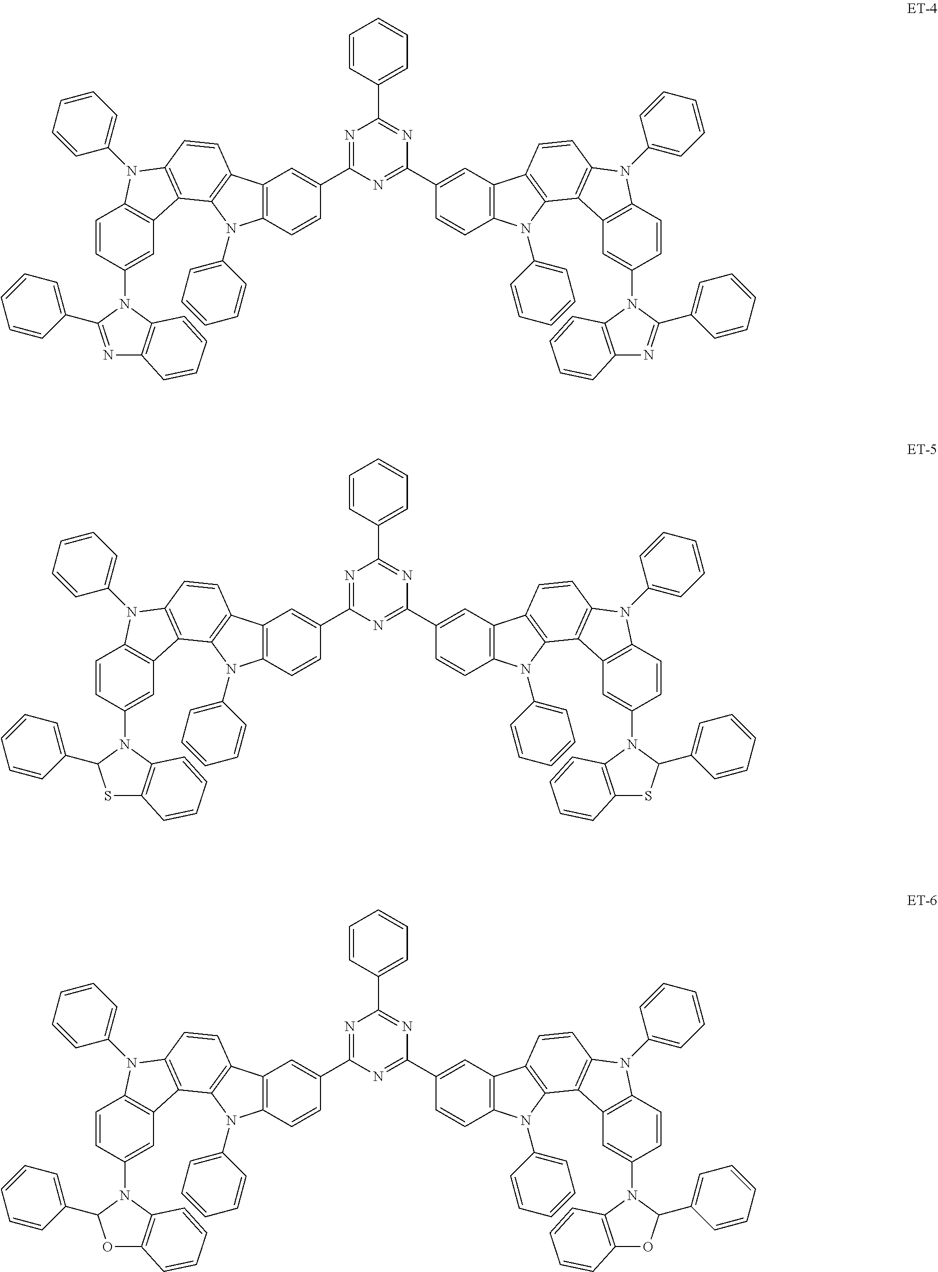

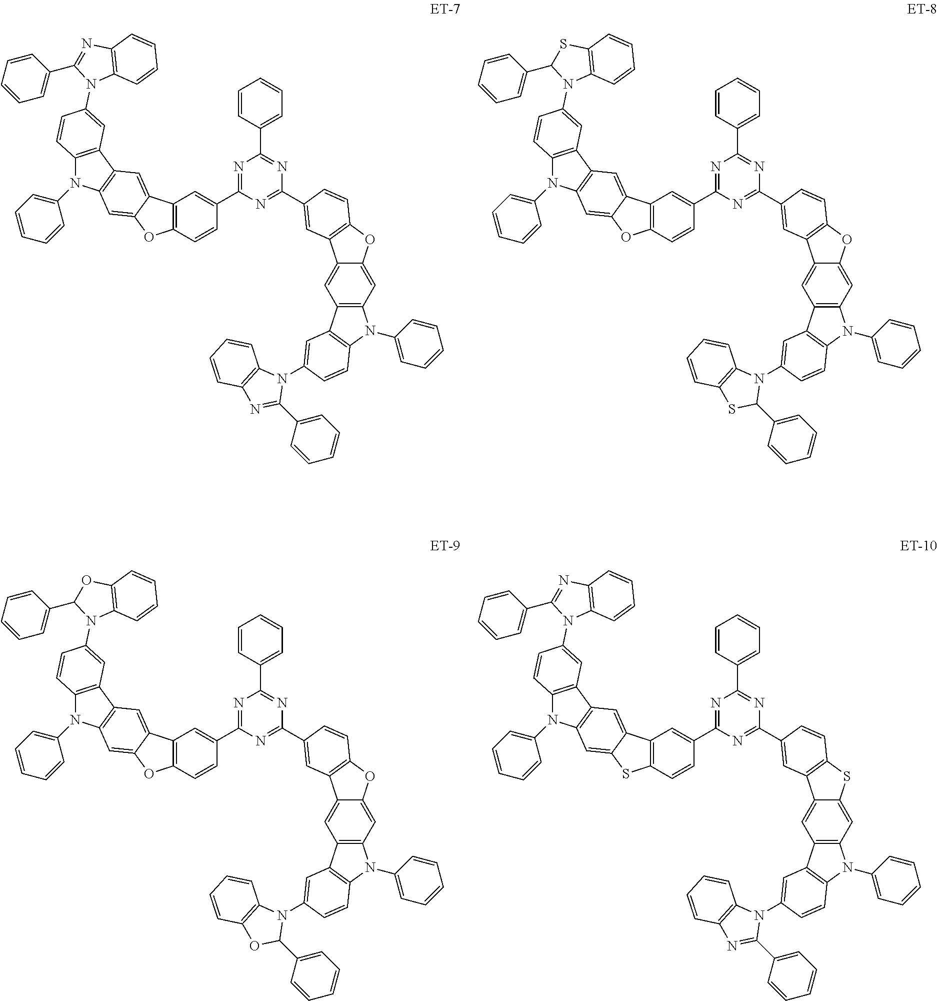

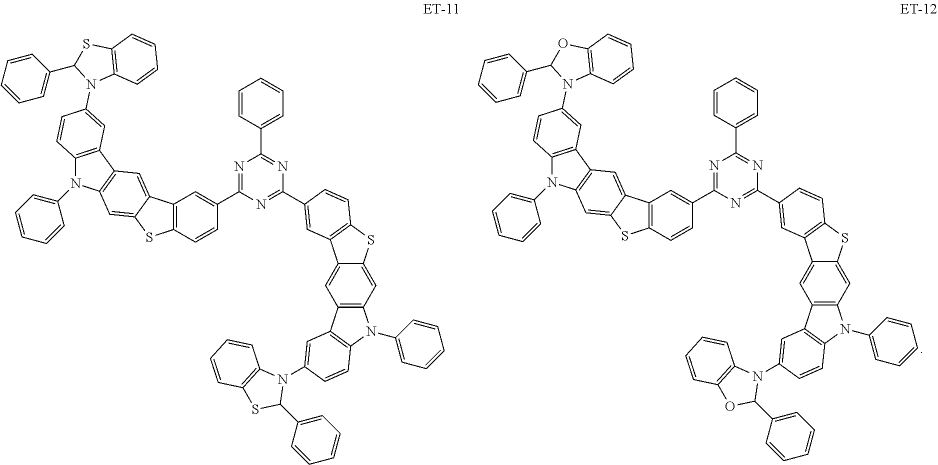 [SMILES: c1ccc(-c2nc(-c3ccc4sc5cc6c(cc5c4c3)c3cc(N4c5ccccc5SC4c4ccccc4)ccc3n6-c3ccccc3)nc(-c3ccc4sc5cc6c(cc5c4c3)c3cc(N4c5ccccc5SC4c4ccccc4)ccc3n6-c3ccccc3)n2)cc1 ; c1ccc(-c2nc(-c3ccc4sc5cc6c(cc5c4c3)c3cc(N4c5ccccc5OC4c4ccccc4)ccc3n6-c3ccccc3)nc(-c3ccc4sc5cc6c(cc5c4c3)c3cc(N4c5ccccc5OC4c4ccccc4)ccc3n6-c3ccccc3)n2)cc1]

12. A nitrogen-containing compound represented by Formula 1: 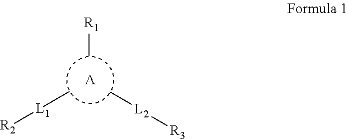
wherein,
in Formula 1,
ring A is a substituted or unsubstituted heterocycle having 3 ring-forming carbon atoms, three ring-forming heteroatoms selected from among a nitrogen atom, an oxygen atom, and a sulfur atom, and at least one ring-forming nitrogen atom,
L₁ and L₂ are each independently a substituted or unsubstituted heteroarylene group having 6 to 30 ring-forming carbon atoms, having at least one ring-forming nitrogen atom,
R₁ is a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms, and
R₂ and R₃ are each independently represented by Formula S-1 or Formula S-2: 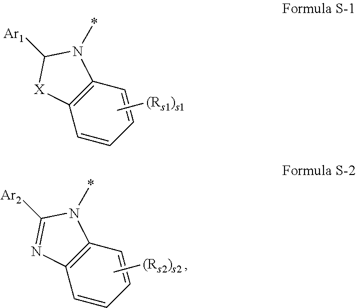
and
wherein,
in Formulas S-1 and S-2,
X is NRₛ₃, O, or S,
Ar₁ and Ar₂ are each independently a substituted or unsubstituted aryl group having 6 to 60 ring-forming carbon atoms, or a substituted or unsubstituted heteroaryl group having 2 to 60 ring-forming carbon atoms,
Rₛ₁ to Rₛ₃ are each independently a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms,
s1 and s2 are each independently an integer of 0 to 4, and [symbol]
is a site linked to Formula 1.

13. The nitrogen-containing compound of claim 12, wherein Formula 1 is represented by Formula 2-1 or Formula 2-2: 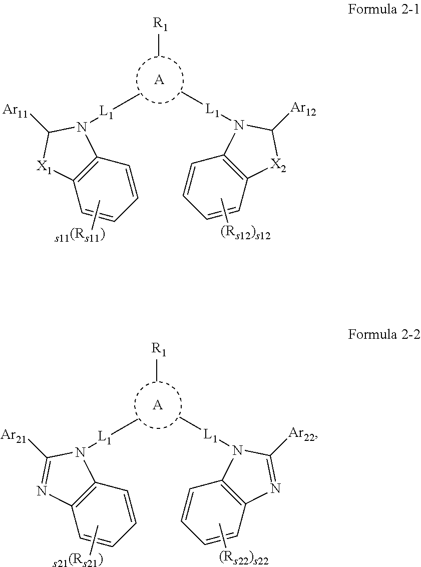
and
wherein,
in Formulas 2-1 and 2-2,
X₁ and X₂ are each independently O or S,
Ar₁₁, Ar₁₂, Ar₂₁, and Ar₂₂ are each independently a substituted or unsubstituted aryl group having 6 to 30 ring-forming carbon atoms,
Rₛ₁₁, Rₛ₁₂, Rₛ₂₁, and Rₛ₂₂ are each independently a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms,
s11, s12, s21, and s22 are each independently an integer of 0 to 4, and
ring A, L₁, L₂, and R₁ are as defined in Formula 1.

14. The nitrogen-containing compound of claim 12, wherein Formula 1 is represented by Formula 3-1 or Formula 3-2: 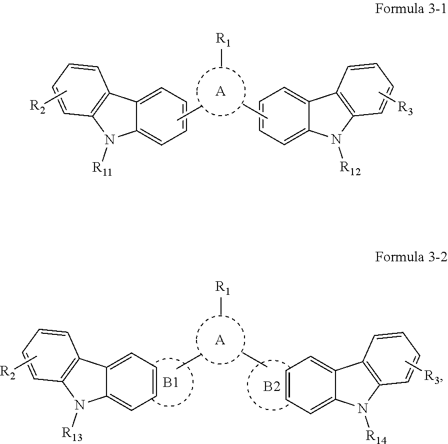
and
wherein,
in Formulas 3-1 and 3-2,
ring B1 and ring B2 are each independently a substituted or unsubstituted hydrocarbon ring having 5 to 30 ring-forming carbon atoms, or a substituted or unsubstituted heterocycle having 2 to 30 ring-forming carbon atoms,
Rᵢ₁, R₁₂, R₁₃, and R₁₄ are each independently a substituted or unsubstituted aryl group having 6 to 30 ring-forming carbon atoms, and
ring A, R₁, R₂, and R₃ are as defined in Formula 1.

15. The nitrogen-containing compound of claim 12, wherein Formula 1 is represented by Formula 4 or Formula 5: 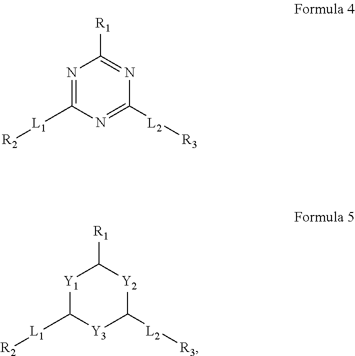
and
wherein,
in Formula 5,
at least one selected from among Y₁ to Y₃ is NR₄, and any remaining Y₁ to Y₃ is each independently O or S,
R₄ is a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms, and
in Formulas 4 and 5,
L₁, L₂, and R₁ to R₃ are as defined in Formula 1.

16. The nitrogen-containing compound of claim 15, wherein Formula 4 is represented by Formula 4-1 or Formula 4-2: 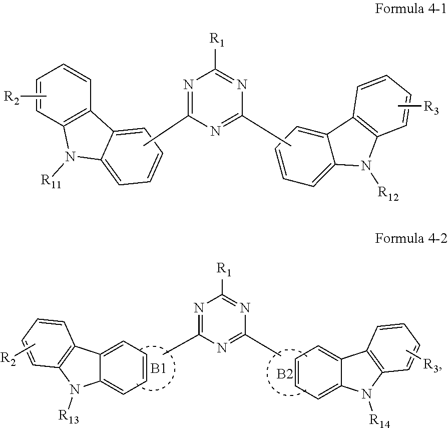
and
wherein,
in Formulas 4-1 and 4-2,
ring B1 and ring B2 are each independently a substituted or unsubstituted hydrocarbon ring having 5 to 30 ring-forming carbon atoms, or a substituted or unsubstituted heterocycle having 2 to 30 ring-forming carbon atoms,
R₁₁, R₁₂, R₁₃, and R₁₄ are each independently a substituted or unsubstituted aryl group having 6 to 30 ring-forming carbon atoms, and
R₁, R₂, and R₃ are as defined in Formula 1.

17. The nitrogen-containing compound of claim 12, wherein L₁ and L₂ are each independently a substituted or unsubstituted divalent carbazole derivative.

18. The nitrogen-containing compound of claim 12, wherein Formula 1 is represented by at least one selected from among the compounds of Compound Group 1:

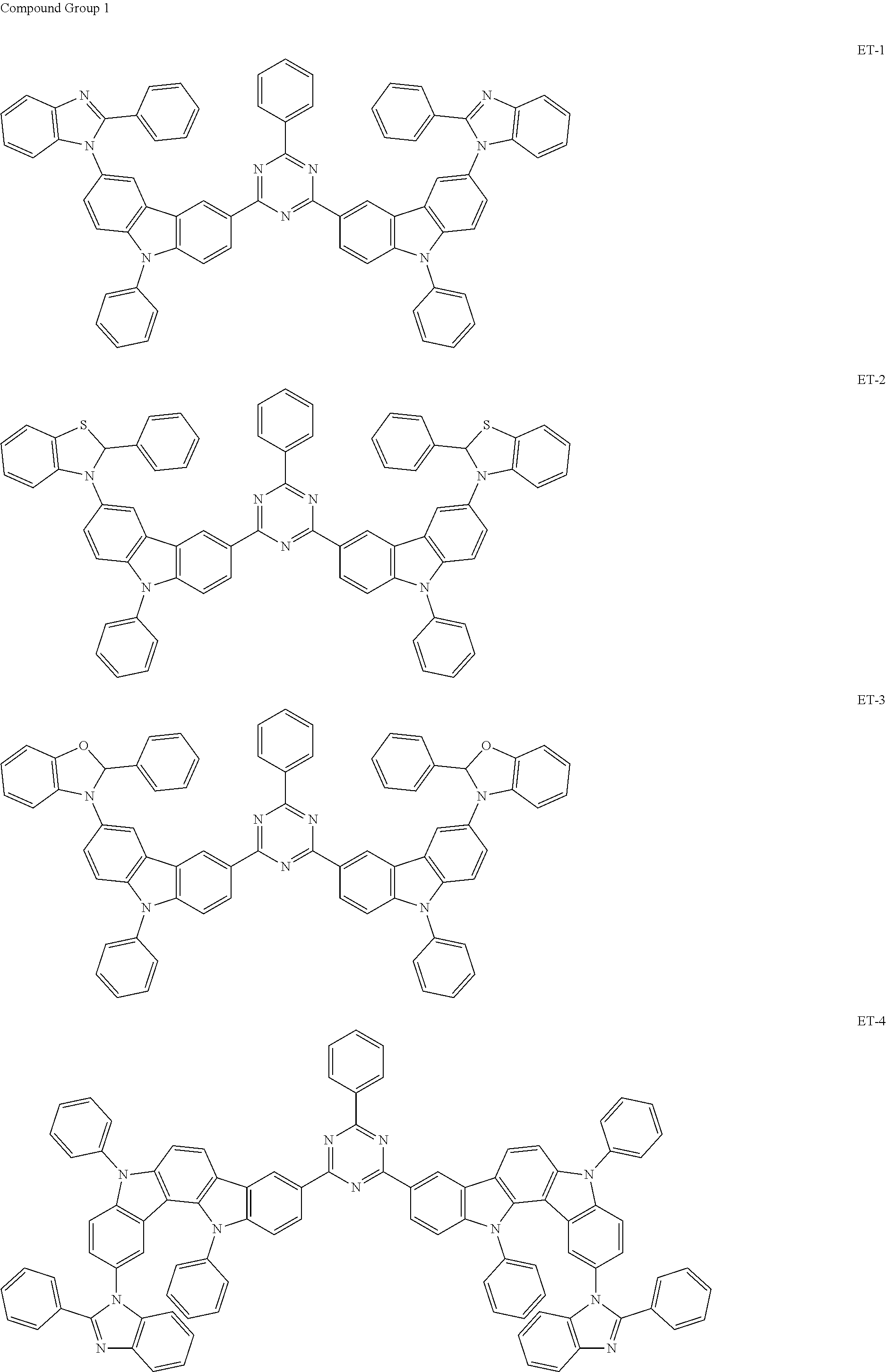

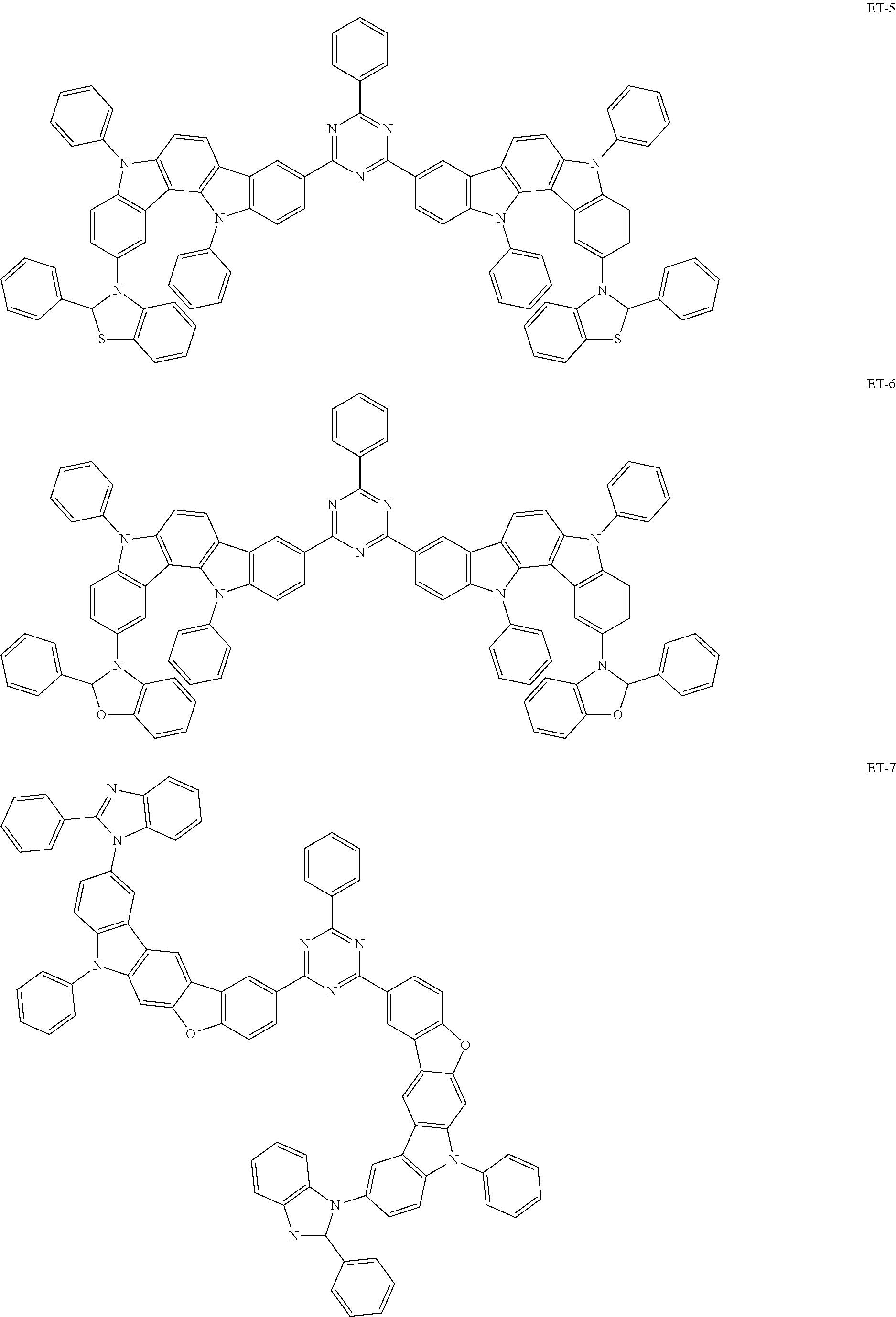

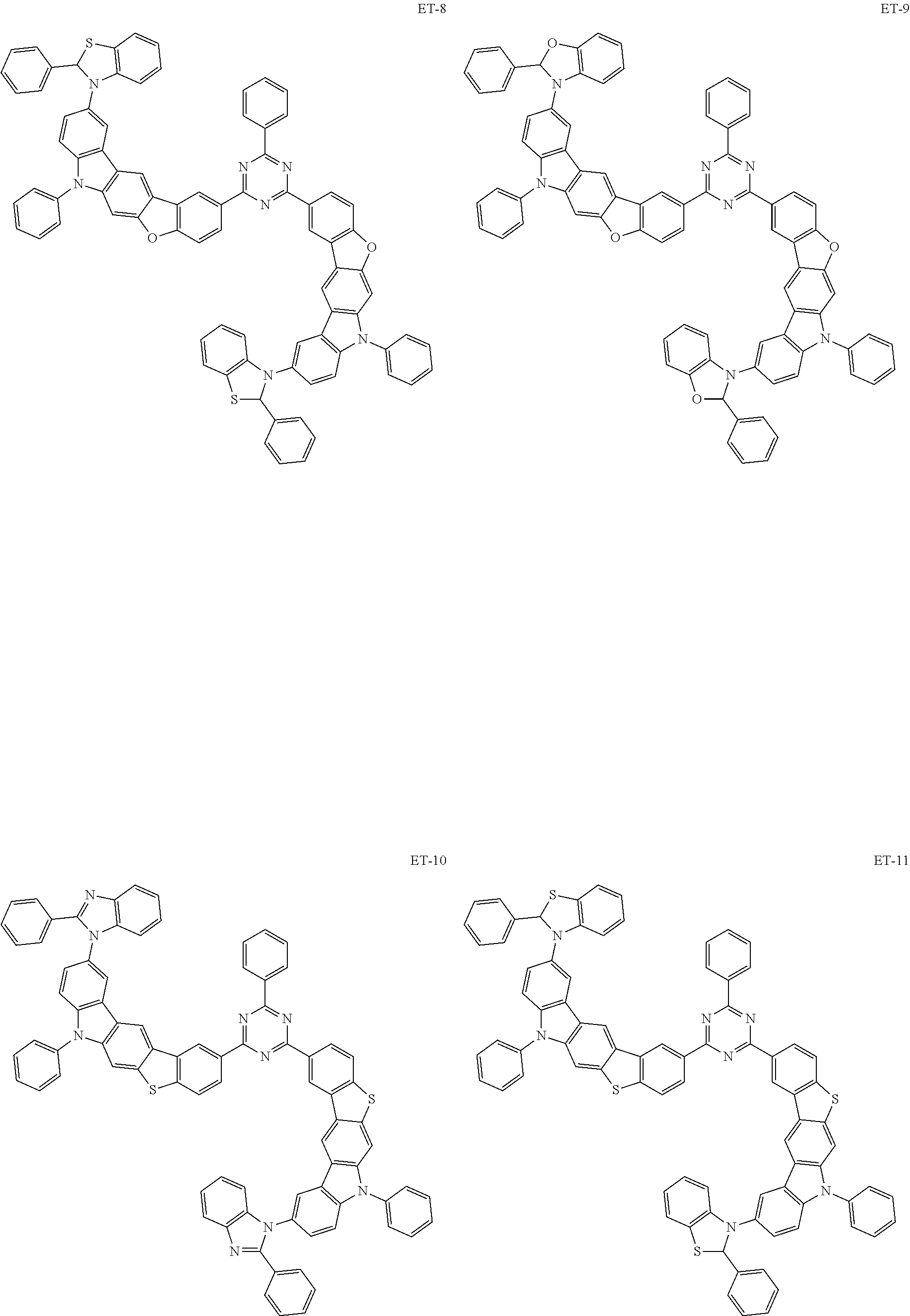

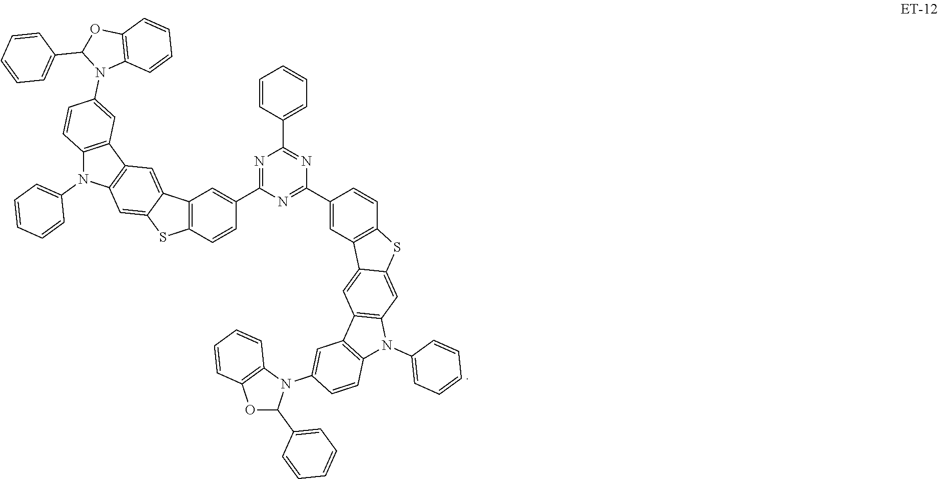 [SMILES: c1ccc(-c2nc(-c3ccc4sc5cc6c(cc5c4c3)c3cc(N4c5ccccc5OC4c4ccccc4)ccc3n6-c3ccccc3)nc(-c3ccc4sc5cc6c(cc5c4c3)c3cc(N4c5ccccc5OC4c4ccccc4)ccc3n6-c3ccccc3)n2)cc1]

19. An electronic device comprising a display device, the display device comprising:
a base layer;
a circuit layer on the base layer; and
a display element layer on the circuit layer and comprising a light emitting element,
wherein,
the light emitting element comprises a first electrode, a second electrode on the first electrode, and an emission layer between the first electrode and the second electrode,
the emission layer comprises a nitrogen-containing compound represented by Formula 1: 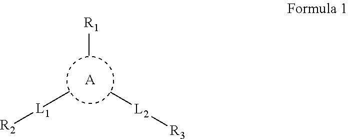
in Formula 1,
ring A is a substituted or unsubstituted heterocycle having 3 ring-forming carbon atoms, three ring-forming heteroatoms selected from among a nitrogen atom, an oxygen atom, and a sulfur atom, and at least one ring-forming nitrogen atom,
L₁ and L₂ are each independently a substituted or unsubstituted heteroarylene group having 6 to 30 ring-forming carbon atoms, having at least one ring-forming nitrogen atom,
R₁ is a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms, and
R₂ and R₃ are each independently represented by Formula S-1 or Formula S-2: 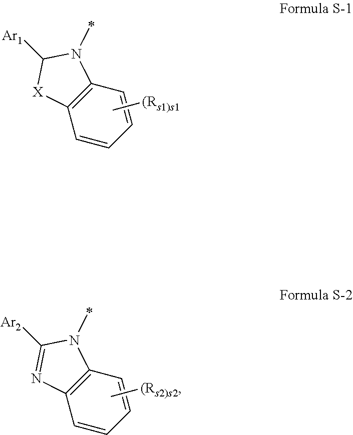
and
wherein, in Formulas S-1 and S-2,
X is NRₛ₃, O, or S,
Ar₁ and Ar₂ are each independently a substituted or unsubstituted aryl group having 6 to 60 ring-forming carbon atoms, or a substituted or unsubstituted heteroaryl group having 2 to 60 ring-forming carbon atoms,
Rₛ₁ to Rₛ₃ are each independently a hydrogen atom, a deuterium atom, a substituted or unsubstituted alkyl group having 1 to 10 carbon atoms, a substituted or unsubstituted aryl group having 6 to 60 ring-forming carbon atoms, or a substituted or unsubstituted heteroaryl group having 2 to 60 ring-forming carbon atoms,
s1 and s2 are each independently an integer of 0 to 4, and [symbol]
is a site linked to Formula 1.

20. The electronic device of claim 19, wherein the display device is selected from among a television, a monitor, an outdoor billboard, a personal computer, a laptop, a personal digital terminal, a vehicle display device, a game console, a portable electronic device, and a camera.
